SEQUENCE LISTING (6 sequences)

SEQ ID NO: 1 - protein, 25 aa, Artificial Sequence
  synthesized
ACDCRGDCFCGGGGIVRRADRAAVP

SEQ ID NO: 2 - protein, 18 aa, Artificial Sequence
  synthesized
IVRRADRAAVPGGGGRGD

SEQ ID NO: 3 - protein, 18 aa, Artificial Sequence
  synthesized
IVRRADRAAVPGGGGRGD

SEQ ID NO: 4 - protein, 11 aa, Artificial Sequence
  synthesized
IVRRADRAAVP

SEQ ID NO: 5 - protein, 14 aa, Artificial Sequence
  synthesized
ACDCRGDCFCGGGG

SEQ ID NO: 6 - protein, 7 aa, Artificial Sequence
  synthesized
GGGGRGD

(19) United States
(12) Patent Application Publication
(10) Pub. No.: US 2022/0202912 A1
(43) Pub. Date: Jun. 30, 2022

(54) Polypeptides in Preparation of Drugs for Treatment or Prevention of Rheumatoid Arthritis

(71) Applicant: Hanmei XU, Nanjing (CN)

(72) Inventors: Hanmei XU, Nanjing (CN); Chunyan Pu, Nanjing (CN); Hong Shen, Nanjing (CN)

(21) Appl. No.: 17/555,483

(22) Filed: Dec. 19, 2021

Related U.S. Application Data
(63) Continuation-in-part of application No. 14/368,850, filed on Jun. 26, 2014, now abandoned.

(30) Foreign Application Priority Data
Dec. 27, 2011 (CN) 201110443067.9

(51) Int. Cl. A61K 38/39; A61K 47/64; A61K 47/60; A61P 19/02

(52) U.S. Cl. CPC A61K 38/39; A61P 19/02; A61K 47/60; A61K 47/643

(57) ABSTRACT

The present invention relates to the pharmaceutical field, specifically to a polypeptidepresenting high integrin affinity and bonding capacity; the polypeptidecan be adopted in prevention and treatment of rheumatoid arthritis. Said polypeptide is: polypeptide III: mPEG-SC₂₀ₖ-Ile-Val-Arg-Arg-Ala-Asp-Arg-Ala-Ala-Val-Pro-Gly-Gly-Gly-Gly-Arg-Gly-Asp (SEQ ID NO: 3). This polypeptide can be adopted in treatmen2_t of rheumatoid arthritis.

### CROSS REFERENCE TO RELATED APPLICATION

This application is a Continuation-In-Part of U.S. application Ser. No. 14/368,850, filed Jun. 26, 2014, titled “POLYPEPTIDES IN PREPARATION OF DRUGS FOR TREATMENT OR PREVENTION OF RHEUMATOID ARTHRITIS”, which is a national stage entry of PCT/CN2012/087442, filed Dec. 25, 2012, which in turn claims priority to Chinese patent application number 201110443067.9 filed Dec. 27, 2011, the content of which is hereby incorporated by reference in their entirety.

### FIELD OF THE INVENTION

The present invention relates to the pharmaceutical field, specifically to the polypeptides used in treatment or prevention of rheumatoid arthritis.

### BACKGROUND OF THE INVENTION

Rheumatoid arthritis (RA) is one of the commonest autoimmune inflammatory arthropathies and major causes for disability. It is a chronic, symmetrical multi-synovial arthritis of unknown etiology. The incidence rate of RA is about 0.5%-1.0% throughout the world and about 0.4% in China. It can attack people at any age, but the risk goes higher with the increase of age. In addition, RA is closely related to gender, and the incidence ratio between male and female is 1:3. The female at the age of 45-55 are at the highest risk. The initial symptoms of RA are progressive pain and swelling in hands and wrists, particularly the swelling at the back of wrists. Though such symptoms can be relieved with common symptomatic treatments, they tend to reappear repeatedly due to irregular or underdosed medication. With the development of the disease, progressive stiffness of joints may appear early in the morning and usually lasts for more than one hour, meanwhile, some joint dysfunctions may also appear. As is mentioned above, the etiology and pathogenesis of rheumatoid arthritis remain unknown, and its basic pathological manifestations include vasculitis and synovitis. When RA attacks, a layer of pannus forms on the synovial membrane due to angiogenesis, which consequently results in thickening of synovial membrane, increase of exudate, release of various cytokines, cartilage destruction and bone erosion. It can also affect surrounding tissues, such as muscular compartments, ligaments, tendon sheaths and muscles, and finally affect the stability of joints and lead to joint deformation and disability. The RA vasculitis may attack other organs throughout the body and manifests itself as a systemic disease.

Currently, drugs for RA treatment can be categorized into two types: symptom-controlling drugs and disease-controlling drugs. The symptom-controlling drugs can be further divided into 4 groups: 1. NSAIDs, long regarded as first-line anti-RA agents; there are more than dozens of NSAIDs available on the Chinese market; 2. glucocorticoids, very good anti-inflammatory agents; but they cannot significantly improve the symptoms and will lead to many serious side effects if being used alone for a long time. They can be used, however, in the short term in moderate dose before the slow-onset agents take effect, and would be necessary to form combined medication with the second-line agents in pulse therapy of RA flare-ups, particularly those patients with extra-articular manifestations; 3. slow-onset, anti-rheumatic drugs, usually regarded as second-line agents and including antimalarials, sodium aurothiomalate (gold), penicillamine and sulfasalazine; they take effect considerably slowly, but have positive functions in improving the overall condition of RA patients. They are also called disease-modifying antirheumatic drugs (DMARDs); 4 immunosuppressants, including methotrexate, cyclophosphamide, azathioprine, tripterygium and sinomenine, etc.

Angiogenesis is one of the main histological characteristics of rheumatoid arthritis. It causes hyperplasia of synovial membrane and infiltration of inflammatory cells—the basis for the formation of pannus and final destruction of joints. Due to angiogenesis, newly-formed blood vessels invade the joint cartilage, which, in healthy condition, contains no blood vessels. The invasion of blood vessels leads to the erosion of cartilage, pain and eventually deformation of the whole joint. Also due to angiogenesis, the thickness of patients' synovial membrane increases. Normally, the inner layer of synovial membrane in a health people constitutes only 1-2 layers of cells, while it would increase to 4-10 layers (sometimes 20 layers) of cells when RA attacks. These increased cells are not only in great quantity, but also extremely active. They can secrete a large quantity of cytokines, signaling molecules and proteases, all of which accelerate the process of joint destruction. In addition, there are a large quantity of inflammatory cells, such as T cells, B cells and monocytes infiltrating in the synovial membrane of RA patients.

Under normal physiological conditions, angiogenesis is strictly regulated and is a necessary process particularly important for reproduction, fetal development, tissue repair and wound healing. However, it takes place under many pathological conditions, including growth and metastasis of tumors, inflammatory disorders such as RA, psoriasis, osteoarthritis, inflammatory bowel disease (IBD, including Crohn's disease and ulcerative colitis) and others.

Integrins are a type of receptors widely found on the cell surface. They can induce cell—cell adhesion as well as adhesion between cells and extracellular matrix; they can also facilitate angiogenesis by means of mediating interaction between intracellular cytoskeletal proteins and extracellular matrix molecules. Currently, at least 8 types of integrins (α1β1, α2β1, α3β1, α6β1, α6≢4, α5β1, ανβ3, ανβ5) have been found closely related to angiogenesis, among them ανβ3 being the most important. As integrin ανβ3 can be expressed in many cell types and combines with a variety of ligands during multicellular activities, it is involved in angiogenesis, infiltration and metastasis of tumors as well as other physiological or pathological processes such as inflammation, wound healing and blood coagulation. Integrin ανβ3 can alsorecognize the Arg-Gly-Asp (RGD) sequence in its ligands, which means that polypeptides bearing the RGD sequence can function as integrin antagonists, and the RGD sequence can be adopted as a vector, targetedly delivering therapeutic polypeptides to the endothelium of newly generated blood vessels so that those diseases involving angiogenesis can be effectively treated. The RGD-bearing, angiogenesis-inhibiting polypeptides can not only block the pathways of oxygen and nutrients to the synovial membrane by inhibiting angiogenesis, but also directly lead to degeneration of blood vessels therein. Therefore, they can inhibit hyperplasia of synovial membrane of RA patients. In short, inhibition of angiogenesis is an essential step for treatment of RA, while related studies show that the proliferation and migration of endothelial cells are two crucial mechanisms for angiogenesis.

Researches have shown that on the one hand the sequence Ile-Val-Arg-Arg-Ala-Asp-Arg-Ala-Ala-Val-Pro (Seq. No.4), the 60-70 amino acids of endostatin, presents high activity in inhibiting angiogenesis under in vitro conditions, even higher than endostatin itself; and on the other hand the polypeptide Ala-Cys-Asp-Cys-Arg-Gly-Asp-Cys-Phe-Cys-Gly-Gly-Gly-Gly (see Seq. No.5) can specifically combine with integrin as the RGD-4C sequence it contains is one of the most important ligands of integrin. In view of these findings, a polypeptide is constructed in the present invention through combining said sequence Ala-Cys-Asp-Cys-Arg-Gly-Asp-Cys-Phe-Cys-Gly-Gly-Gly-Gly (presenting high integrin affinity and bonding capacity due to the RGD-4C sequence it contains) to the N-terminal of said sequence Ile-Val-Arg-Arg-Ala-Asp-Arg-Ala-Ala-Val-Pro (presenting high angiogenesis-inhibiting effect). The amino acid sequence of this constructed polypeptide (namely, polypeptide I) is: Ala-Cys-Asp-Cys-Arg-Gly-Asp-Cys-Phe-Cys-Gly-Gly-Gly-Gly-Ile-Val-Arg-Arg-Ala-Asp-Arg-Ala-Ala-Val-Pro. It contains 25 amino acids in total, and presents high affinity and bonding capacity to integrin (specifically ανβ3) on the one hand and high angiogenesis-inhibiting effect on the other, as it simultaneously integrates the RGD-4C sequence and the angiogenesis-inhibiting sequence (Seq. No.4). The prior patent “Highly Effective Angiogenesis-inhibiting Polypeptide and the Preparation and Uses Thereof” (ZL200510040378.5) disclosed the therapeutic effect of this sequence in treatment of melanoma, however, it didn't disclose the therapeutic effect of this sequence in treatment of other diseases. On the basis of a great number of experiments, the inventor of the present invention has found out that the polypeptide I presents prominent therapeutic effect on collagen-induced arthritis (CIA) and adjuvant-induced arthritis (AIA), along with fewer side effects, less dose and lower production cost in comparison with other conventional methods. Polypeptide I disclosed in the present invention is reasonably designed, presenting high feasibility in production and high effect in treatment of rheumatoid arthritis. It enormously expands the therapeutic spectrum of this series of integrin-blockers, not only providing a new perspective for developing drugs of the same kind, but also highlighting their social benefits and great market potential.

The prior patent “Highly Effective Angiogenesis-inhibiting Polypeptide and the Preparation and Uses Thereof” (ZL 200510040378.5) also disclosed another polypeptide (namely, polypeptide II), the sequence of which is Ile-Val-Arg-Arg-Ala-Asp-Arg-Ala-Ala-Val-Pro-Gly-Gly-Gly-Gly-Arg-Gly-Asp. This polypeptide contains an integrin ligand sequence:Gly-Gly-Gly-Gly-Arg-Gly-Asp (Seq. No.6) and an angiogenesis-inhibiting sequence: Ile-Val-Arg-Arg-Ala-Asp-Arg-Ala-Ala-Val-Pro; wherein the RGD (Arg-Gly-Asp) sequence contained in said integrin ligand sequence can realize effective bonding between the whole polypeptide sequence and the integrin subtype, while said angiogenesis-inhibiting sequence can effectively inhibit the process of angiogenesis. The prior patent focused only on the effect of polypeptide II on treating melanoma, in contrast, the inventor of the present invention, on the basis of further studies on polypeptide II, found out its therapeutic effect on rheumatoid arthritis, which consequently broadens its indication range and highlights its social benefits and market potential. In addition, in the present invention, PEG modification of polypeptide II is adopted to construct polypeptide III: mPEG-SC₂₀ₖ-Ile-Val-Arg-Arg-Ala-Asp-Arg- Ala-Ala-Val-Pro-Gly-Gly-Gly-Gly-Arg-Gly-Asp. The application of polypeptide III in preparation of drugs for angiogenesis-caused eye diseases was disclosed in the prior patent:ZL 201110128464.7; in contrast, the inventor of the present invention, on the basis of further studies on polypeptide III, found out its great therapeutic effect on rheumatoid arthritis, which consequently broadens its indication range and highlights its social benefits and market potential.

### SUMMARY OF THE INVENTION

### Objective of the Invention

On the basis of a large number of researches, the present invention found out that three polypeptides, namely, polypeptide I: Ala-Cys-Asp-Cys-Arg-Gly-Asp-Cys-Phe-Cys-Gly-Gly-Gly-Gly-Ile-Val-Arg-Arg-Ala-Asp-Arg-Ala-Ala-Val-Pro (two pairs of disulfide bonds contained in the sequence are pairing in the pattern of 1-4 and 2-3), polypeptide II: Ile-Val-Arg-Arg-Ala-Asp-Arg-Ala-Ala-Val-Pro-Gly-Gly-Gly-Gly-Arg-Gly-Asp and polypeptide III: mPEG-SC₂₀ₖ-Ile-Val-Arg-Arg-Ala-Asp-Arg-Ala-Ala-Val-Pro-Gly-Gly-Gly-Gly-Arg-Gly-Asp present great effect in treatment and prevention of rheumatoid arthritis.

### Technical Solutions

The application of polypeptides in preparation of drugs for treatment or prevention of rheumatoid arthritis, wherein the respective amino acid sequences of said polypeptides are:

polypeptide I: Ala-Cys-Asp-Cys-Arg-Gly-Asp-Cys-
Phe-Cys-Gly-Gly-Gly-Gly-Ile-Val-Arg-Arg-Ala-
Asp-Arg-Ala-Ala-Val-Pro
(AP25 for short, see Seq. No. 1);
polypeptide II: Ile-Val-Arg-Arg-Ala-Asp-Arg-Ala-
Ala-Val-Pro-Gly-Gly-Gly-Gly-Arg-Gly-Asp
(HM-3 for short, see Seq. No. 2);
polypeptide III: mPEG-SC₂₀ₖ-Ile-Val-Arg-Arg-Ala-
Asp-Arg-Ala-Ala-Val-Pro-Gly-Gly-Gly-Gly-Arg-
Gly-Asp (PEG-HM-3 for short, see Seq. No. 3);

said polypeptide I, polypeptide II and polypeptide III are obtained by means of solid-phase synthesis or recombination of expression vectors;
said polypeptide I, polypeptide II and polypeptide III can covalently couple to an adjuvant; said adjuvant can be bovine serum albumin, human serum albumin or polyethylene glycol (PEG);
said drugs for treatment or prevention of rheumatoid arthritis contain an effective amount of salts acceptable to polypeptide I, polypeptide II and polypeptide III, or if necessary, pharmaceutically acceptable vectors or excipients;
said drugs for treatment or prevention of rheumatoid arthritis can be administered through many routes, such as hypodermic injection, intramuscular injection, intravenous injection or drip, oral administration (in the form of pills, capsules, etc.) and nasal spray.

### Beneficial Effects

1. The prior patents have disclosed that polypeptide I, polypeptide II and polypeptide III have anti-tumor effect; the present invention, on the basis of further studies on polypeptide I, polypeptide II and polypeptide III, has found out that polypeptide I, polypeptide II and polypeptide III present also great therapeutic effect on rheumatoid arthritis (RA); the anti-RA effect of polypeptide I, polypeptide II and polypeptide III has never been disclosed in any academic document or patent application. This means that the anti-RA effect of polypeptide I, polypeptide II and polypeptide III disclosed in the present invention constitutes a crucial discovery on the treatment of rheumatoid arthritis. It not only broadens the indication range of these polypeptides, but also highlights their social benefits and market potential.

2. Introduction to the Pharmacological Mechanism of the Polypeptides

Researches have indicated that on the one hand the sequence Ile-Val-Arg-Arg-Ala-Asp-Arg-Ala-Ala-Val-Pro presents great angiogenesis-inhibiting activity, and on the other the polypeptide Ala-Cys-Asp-Cys-Arg-Gly-Asp-Cys-Phe-Cys-Gly-Gly-Gly-Gly (Seq. No.5) can specifically combine with integrin as the RGD-4C sequence it contains is one of the most important ligands of integrin. In view of these findings, a polypeptide is constructed in the present invention through combining said sequence Ala-Cys-Asp-Cys-Arg-Gly-Asp-Cys-Phe-Cys-Gly-Gly-Gly-Gly (presenting high integrin affinity and bonding capacity due to the RGD-4C sequence it contains) to the N-terminal of the sequence Ile-Val-Arg-Arg-Ala-Asp-Arg-Ala-Ala-Val-Pro (presenting high angiogenesis-inhibiting effect); the polypeptide so constructed, namely, polypeptide I, presents high affinity and bonding capacity to integrin. The amino acid sequence of this integrin-blocking polypeptide is: Ala-Cys-Asp-Cys-Arg-Gly-Asp-Cys-Phe-Cys-Gly-Gly-Gly-Gly-Ile-Val-Arg-Arg-Ala-Asp-Arg-Ala-Ala-Val-Pro. As the RGD-4C sequence it contains presents high affinity and bonding capacity to integrin (specifically ανβ3), it can targetedly deliver the polypeptide to integrin ανβ3; as it also contains an angiogenesis-inhibiting sequence (Seq. No. 4), polypeptide I can simultaneously inhibit the process of angiogenesis.

The sequence Ile-Val-Arg-Arg-Ala-Asp-Arg-Ala-Ala-Val-Pro-Gly-Gly-Gly-Gly-Arg-Gly-Asp, namely, polypeptide II, contains an integrin ligand sequence (Gly-Gly-Gly-Gly-Arg-Gly-Asp) and an angiogenesis-inhibiting sequence (Ile-Val-Arg-Arg-Ala-Asp-Arg-Ala-Ala-Val-Pro); the RGD (Arg-Gly-Asp) sequence contained within said integrin ligand sequence can realize effective bonding between the whole polypeptide sequence and the integrin subtypes, while the angiogenesis-inhibiting sequence contained in polypeptide II can effectively inhibit the process of angiogenesis. In addition, in the present invention, PEG modification of polypeptide II is adopted to construct polypeptide III: mPEG-SC₂₀ₖ-Ile-Val-Arg-Arg-Ala-Asp-Arg-Ala-Ala-Val-Pro-Gly-Gly-Gly-Gly-Arg-Gly-Asp, which extends the half-life period of said polypeptide sequence; As the RGD sequence contained in polypeptide III can targetedly combine with integrin, it can realize specific combination between polypeptide III and the endothelium of newly generated blood vessels within the joint pannus. Meanwhile, the angiogenesis-inhibiting sequence in polypeptide III can inhibit the process of angiogenesis therein, which consequently realizes desirable effect in prevention and treatment of rheumatoid arthritis.

With a large quantity of experiments, the inventor of the present invention found out that polypeptide I, polypeptide II and polypeptide III can effectively inhibit the development of adjuvant-induced RA in rats and collagen-induced RA in DBA/1 mice. The in vivo experiments have proved that this series of polypeptides have prominent effect in treatment of RA; they also demonstrate such advantages as little side effect, small effective dose and low production cost. The method for preparing the integrin-blocking polypeptides disclosed in the present invention is reasonable in design and presents high feasibility; and drugs prepared with this method can be applied in prevention and treatment of rheumatoid arthritis. This enormously expands the therapeutic spectrum of these integrin blockers, which on the one hand provides a new perspective for developing anti-RA drugs of the same kind, and on the other highlights their social profits and market potential.

3. Polypeptide III has the effect of relieving arthritis, mainly through dual functions of antiangiogenesis and anti-inflammation. It can reduce the activity of macrophages, inhibit their secretion of pro-inflammatory and proangiogenic-related cytokines, and relieve inflammation; inhibit the expression and release of TNF-α by macrophages and the expression of VEGF by vascular endothelial cells, inhibit synovitis and hyperplasia of synovial membrane, and inhibit the formation of pannus; and at the same time, Polypeptide III play an anti-inflammatory effect by inhibiting inflammatory signaling pathways of integrin ανβ3, VEGF, TNF-α, NF-κB and NO.

### BRIEF DESCRIPTION OF DRAWINGS

FIG. 1 polypeptide I in improving paw swelling of CIA (DBA/1) mice

FIG. 2 polypeptide I in improving joint swelling of CIA (DBA/1) mice

FIG. 3 polypeptide I in improving joint scoring of CIA (DBA/1) mice

FIG. 4 polypeptide I in improving primary swelling of left paws of AIA rats

FIG. 5 polypeptide I in improving secondary swelling of right paws of AIA rats

FIG. 6 polypeptide I in improving joint scoring of AIA rats

FIG. 7 polypeptide II in improving paw swelling of CIA (DBA/1) mice

FIG. 8 Polypeptide II in improving joint swelling of CIA (DBA/1) mice

FIG. 9 polypeptide II in improving joint scoring of CIA in DBA/1 mice

FIG. 10 polypeptide II in improving primary swelling of left paws of CIA in rats

FIG. 11 polypeptide II in improving secondary swelling of right paws of AIA rats

FIG. 12 polypeptide II in improving joint scoring of AIA rats

FIG. 13 polypeptide III in improving paw swelling of CIA (DBA/1) mice

FIG. 14 polypeptide III in improving joint swelling of CIA (DBA/1) mice

FIG. 15 polypeptide III in improving joint scoring of CIA in DBA/1 mice

FIG. 16 polypeptide III in improving primary swelling of left paws of AIA rats

FIG. 17 polypeptide III in improving secondary swelling of right paws of AIA rats

FIG. 18 polypeptide III in improving joint scoring AIA rats

FIG. 19 RNA integrity and cDNA contamination test by Denaturing Agarose Gel Electrophoresis. Lane 1: Total RNA of test group; Lane 2: Total RNA of negative group.

FIG. 20 Box-plot normalized intensity values between test group and negative group. H: negative group; M: test group.

FIG. 21 The scatter-plot is a result of gene expression variation between the test group and negative group. H: negative group; M: test group.

FIG. 22 Heat map and hierarchical clustering of gene expression variation between the test group and negative group. H: negative group; M: test group.

FIG. 23 Pathway analysis of gene expression variation between the test group and negative group. 1): regulation up, negative group& test group; 2): regulation down, negative group& test group.

FIG. 24 Flow cytometric analysis of Macrophage apoptosis dealt with different drug.

FIG. 25a The effect of mPEG-SC₂₀ₖ-HM-3 on the expression of TNFR1, TNFR2, NF-κB and TNF-proteins in macrophage. V: control; M₁: PMA induced; A₁₋₃: Adalimumab (100, 400, 800 μg/mL); P₁₋₄: mPEG-SC₂₀ₖ-HM-3 (4.5, 9, 18, 36 μM).

FIG. 25b Gray analysis of mPEG-SC₂₀ₖ-HM-3 on the relative expression of TNFR1, TNFR2, NF-κB and TNF-α proteins in activated U937 cells. A: TNFR1 protein; B: TNFR2 protein; C: NF-κB (p65) protein; D: TNF-α protein. Ad: Adalimumab (m/mL); Model 1: LPS induced; Model 2: PMA induced; Model 3: PMA+LPS.

FIG. 26a Effect of mPEG-SC₂₀ₖ-HM-3 on integrin α5, β1 protein expression of macrophage, VEGFR2 protein and HIF-1β protein, IL-6 protein expression. V: control; M₁: PMA induced; M₂: PMA+LPS; M₃: LPS; A₁₋₃: Adalimumab (100, 400, 800 μg/mL); P₁₋₆: mPEG-SC₂₀ₖ-HM-3 (4.5, 9, 13.5, 18, 36, 72 μM).

FIG. 26b The relative inhibition rate of mPEG-SC₂₀ₖ-HM-3 (PEG-HM-3) on the expression of integrin α5, β1 protein, VEGFR2 receptor protein and HIF-1β and IL-6 protein in macrophage. A: integrin α5; B: integrin β1; C: HIF-1β in 24 h; D: HIF-1β in 48 h; E: VEGFR₂; F: IL-6. Model₁: PMA; Model₂: PMA+LPS.

FIG. 27a The effect of mPEG-SC₂₀ₖ-HM-3 on the expression of TLR-4 protein in activated U937 cells. V: control; A₁₋₂: Adalimumab (50, 100 μg/mL); P₁₋₆: mPEG-SC₂₀ₖ-HM-3 (2.25, 4.5, 7, 9, 13.5, 18 μM); M₁:PMA induced; M₂: PMA+LPS induced; M₃: LPS induced

FIG. 27b The relative expression of TLR-4 protein in activated U937 cells. Ad: Adalimumab (m/mL); Model 1: PMA induced; Model 2: PMA+LPS induced; Model 3: LPS induced. Compared with the control, #p<0.05; compared with the model group, *p<0.05, **p<0.01 or ***p<0.001.

FIG. 28 Western blot analysis of the expression level of integrin aα, β3, α5, β1 in Macrophage after treatment. Lane 1: control; Lane 2-6: treatment with mPEG-SC₂₀ₖ-HM-3 at the dose of 0.5, 1.0, 2.0, 4.0, 8.0 μmol/L. (β-actin as the control).

FIG. 29 Western blot analysis of the expression level of MEK1, p-MEK1, AKT1, p-AKT1, ERK1/2, p-ERK1/2 and VEGF in HUVECs after treatment (A-G). Lane 1: control; Lane 2-6: treatment with mPEG-SC₂₀ₖ-HM-3 at the dose of 0.5, 1.0, 2.0, 4.0, 8.0 μmol/L. (β-actin as the control).

FIG. 30 The mechanism of mPEG-SC₂₀ₖ-HM-3 in anti-RA.

FIG. 31 The impact of mPEG-SC₂₀ₖ-HM-3 on the secretion of IL-1β from macrophage. The optical density at 450 nm can reflect the content of IL-1β. The mPEG-SC₂₀ₖ-HM-3 showed dose-dependent inhibitory effect on the secretion of IL-1β from macrophage. *p<0.05, **p<0.01 vs model group.

FIG. 32 Inhibition rate at the 8^{th} day according to granuloma weight. G1: Model; G2: Dex (5 mg/kg, once a day); G3: mPEG-SC₂₀ₖ-HM-3 (20 mg/kg, once two days); G4: mPEG-SC₂₀ₖ-HM-3 (10 mg/kg, once two days); G5: mPEG-SC₂₀ₖ-HM-3 (5 mg/kg, once two days). *p<0.05, **p<0.01 vs model group.

FIG. 33 Added value of weight at the 8^{th} day. G1: model; G2: Dex (5 mg/kg, once a day); G3: mPEG-SC₂₀ₖ-HM-3 (20 mg/kg, once two days); G4: mPEG-SC₂₀ₖ-HM-3 (10 mg/kg, once two days); G5: mPEG-SC₂₀ₖ-HM-3 (5 mg/kg, once two days). *p<0.05, **p<0.01 vs model group.

FIG. 34 Cotton ball at the 8^{th} day from the rats. G1: model; G2: Dex (5 mg/kg, once a day); G3: mPEG-SC₂₀ₖ-HM-3 (5 mg/kg, once two days); G4: mPEG-SC₂₀ₖ-HM-3 (10 mg/kg, once two days); G5: mPEG-SC₂₀ₖ-HM-3 (20 mg/kg, once two days).

FIG. 35 Ankle swelling changes of left toe over time in TNF-luc mouse.

FIG. 36 Foot swelling changes of left toe over time in TNF-luc mouse.

FIG. 37 Left toe swelling changes over time in TNF-luc mouse.

FIG. 38 The expression changes of TNF-α in acute inflammation of transgenic TNF-luc mice induced by λ-carrageenan during 72 h.

FIG. 39 The changes of fluorescence intensity in vivo imaging of λ-carrageenan transgenic TNF-luc mice.

FIG. 40 Ankle swelling changes of primary toes over time in TNF-luc mice (n=2).

FIG. 41 Ankle swelling changes of secondary toes over time in TNF-luc mice (n=2).

FIG. 42 Foot swelling changes of primary toes over time in TNF-luc mice (n=2).

FIG. 43l Foot swelling changes of secondary toes over time in TNF-luc mice (n=2).

FIG. 44 Primary toes swelling changes over time in TNF-luc mice (n=2).

FIG. 45 Secondary toes swelling changes over time in TNF-luc mice (n=2).

FIG. 46 Arthritis index score changes over time in TNF-luc mice (n=2).

FIG. 47. Whole body score changes over time in TNF-luc mice (n=2).

FIG. 48. Added value of weight changes over time in TNF-luc mice (n=2).

FIG. 49 The changes of expression of TNF-α in AIA transgenic TNF-luc mice in vivo (n=2).

FIG. 50 HE staining of left and right ankle joints in AlAtransgenic TNF-luc mice in 10×. A-D: left ankle joints. E-H: right ankle joints. A, E: normal group; B, F: model group; C, G: Adalimumab (8 mg/kg) group; D, H: mPEG-SC₂₀ₖ-HM-3 (20 mg/kg). I: HE staining in ankle (**p<0.01).

FIG. 51 Immunohistochemical images of TNF-α expression in the ankle joints of AIA transgenic mice in 10×. A: control; B: model; C: Adalimumab (8 mg/kg); D: mPEG-SC₂₀ₖ-HM-3 (20 mg/kg).

FIG. 52 Immunohistochemical evaluation of TNF-α expression in the ankle joints of AIA transgenic mice. Compared with Model group (0.828±0.255), there was very significant difference in Adalimumab group (8 mg/kg) (0.246±0.110) and mPEG-SC₂₀ₖ-HM-3 (20 mg/kg) (0.149±0.047) (***p<0.001).

FIG. 53 Immunohistochemical images of IL-6 expression in the spleens of AIA transgenic mice in 40×. A: control; B: model; C: Adalimumab (8 mg/kg); D: mPEG-SC₂₀ₖ-HM-3 (20 mg/kg).

FIG. 54 Immunohistochemical evaluation of IL-6 expression in the spleens of AIA transgenic mice. Compared with model group (0.001±0.001), there was very significant difference in Adalimumab group (8 mg/kg) (0.013±0.010) and mPEG-SC₂₀ₖ-HM-3 (20 mg/kg) (0.006±0.002) (***p<0.001).

FIG. 55 Immunohistochemical images of IL-6 expression in the thymus of AIA transgenic mice in 40×. A: control; B: model; C: Adalimumab (8 mg/kg); D: mPEG-SC₂₀ₖ-HM-3 (20 mg/kg).

FIG. 56 Immunohistochemical evaluation of IL-6 expression in the thymus of AIA transgenic mice. Compared with model group (0.281±0.044), there was very significant difference in adalimumab group (8 mg/kg) (0.039±0.041) and mPEG-SC₂₀ₖ-HM-3 (20 mg/kg) (0.068±0.033) (***p<0.001).

FIG. 57 The changes of left ankle joint swelling in CIA transgenic TNF-luc mice (n=2).

FIG. 58 The changes of right ankle joint swelling in CIA transgenic TNF-luc mice (n=2).

FIG. 59 The changes of joint index in CIA transgenic TNF-luc mice (n=2).

FIG. 60 The changes of systemic score in CIA transgenic TNF-luc mice (n=2).

FIG. 61 The changes of weight gain in CIA transgenic TNF-luc mice (n=2).

FIG. 62 The images of TNF-α0 expression changes in vivo in CIA transgenic TNF-luc mice

FIG. 63 The changes of fluorescence intensity in vivo imaging in CIA transgenic TNF-luc mice.

FIG. 64 Immunohistochemical images of TNF-α expression in the ankle joints of CIA transgenic mice in 10×

FIG. 65 Immunohistochemical evaluation of TNF-α expression in the ankle joints of CIA transgenic mice. Compared with control group (###p<0.001); Compared with model group (***p<0.001).

FIG. 66 Immunohistochemical images of IL-6 expression in the spleens of CIA transgenic mice in 40×

FIG. 67 Immunohistochemical evaluation of IL-6 expression in the spleens of CIA transgenic mice. Compared with control group (###p<0.001); Compared with model group (***p<0.001)

FIG.68 Immunohistochemical images of IL-6 expression in the thymus of CIA transgenic mice in 40×

FIG. 69 Immunohistochemical evaluation of IL-6 expression in the thymus of CIA transgenic mice.

### SPECIFIC EMBODIMENTS

### Mechanism Study

The gene changes of human macrophages under the action of mPEG-SC₂₀ₖ-HM-3 were analyzed by using a whole genome array. One-time analysis and comparison of differential gene expression abundance features comprehensiveness, directivity and good screening. It is convenient for us to grasp the overall difference, and select unknown polypeptide targets and signaling pathways, which facilitates in-depth study of drug mechanisms in the future. 19 genes related to RA inflammatory angiogenesis are selected. The up-regulated ones are: cell matrix (MMP3\7\8\10\12\19), and cell metabolism (MTSS1, AMICA1, GALM), and the down-regulated ones are: cytoskeleton (CKAP2L), cell adhesion-related (RASGRP2), angiogenesis-related (ECE1, PTPRU, THBS4, AMOT, VEGFE, PDGF), cell senescence-related (MTFR2), and metabolism-related (LRP3). 29 genes related to RA inflammation are selected. The up-regulated ones are: inflammation-related (IL-1RN, IL-10, IL22RA2) and apoptosis-related (TRAF1, TNFRSF1B, TNFRSF9, TNFRSF11A, TNFSF13\14, 15); and the down-regulated ones are: cell adhesion, migration-related (PODXL), cell proliferation, differentiation-related (CENPQ, NRARP, HIRA, FGFBP3), chemokines (CXCL1\5, CCL3\20), inflammation-related (IL-1A, COX18, BCL2\11A, BCX, MVB12B, TRAIP, HEST), and rheumatoid-related (FGFBP3, OMD). Pathway analysis indicates that mPEG-SC₂₀ₖ-HM-3 has an inhibitory effect on Cytokine-cytokine receptor interaction, Chemokine signaling pathway, TNF signaling pathway, Rheumatoid arthritis, Osteoctast differentiation, and NF-kB pathway.

Through in-depth study of the mechanism of action of mPEG-SC₂₀ₖHM-3, Western blot test results show that under the action of mPEG-SC₂₀ₖ-HM-3, the expression of MEK1, p-MEK1, AKT1, p-AKT1, ERK1/2, p-ERK1/2 in HUVECs and HIF-1β and IL-6 protein factors in macrophages is down-regulated.

To verify its impact on the apoptotic signaling pathway, flow cytometric apoptosis test shows that mPEG-SC₂₀ₖ-HM-3 can induce early and late apoptosis of macrophages. At the same time, Western blot test of NF-kB and the apoptosis pathway shows that the expression of NF-kB, TNFR1, Caspase3, TNF-α protein molecules is down-regulated under the action of mPEG-SC₂₀ₖ-HM-3, and it is found that it can inhibit the activity of macrophages and the release of pro-inflammatory factors, finally exerting the anti-RA effect.

### 1. Test Method for a Human Whole Genome Array

### 1.1 Sample Preparation and RNA Extraction

1.1.1 U937 cells are cultured in a 6-well plate at 3×10⁶ cells/mL per well overnight, and PMA (25 ng/mL) is added on the next day for induction for 48 h. After the cells adhere, cultured supernatant is pipetted away. The cells are treated with mPEG-SC₂₀ₖ-HM-3 (test group) or a medium (negative control group) at 13.5 μmol/L at 37° C. for 48 h.

1.1.2 1×10⁶ cells are taken from each of two groups of samples, and the solution has a weight of 1000 g, and is centrifuged for 5 min. The supernatant is removed. 1 mL of TRIZOL lysis solution is added and mixed uniformly, and the solution stands at room temperature for 5 min.

1.1.3 0.2 mL of chloroform is added to every milliliter of TRIZOL reagent-homogenized sample, and the solution is shaken with force for full emulsification, so that the solution looks like a transparent gel with no phase separation. The solution stands at room temperature for 3 min.

1.1.4 12000 g of solution is centrifuged at 4° C. for 15 min.

1.1.5 A centrifuge tube is taken out carefully, and the supernatant with a volume being about a half of the initial dose of the TRIZOL reagent is pipetted to another RNase-free centrifuge tube.

1.1.6 Isopropanol is added into the supernatant, and the liquid in the centrifuge tube is gently reversed for uniform mixing and stands at room temperature for 30 min.

1.1.7 12000 g of solution is centrifuged at 4° C. for 10 min.

1.1.8 The supernatant is carefully pipetted and discarded, and 1 mL of 75% ethanol is slowly added along the tube wall. The tube wall of the centrifuge tube is gently reversed for washing, and the ethanol is carefully pipetted and discarded.

1.1.9 Then 1 mL of 75% ethanol is added and the solution is briefly eddied on a vortex oscillator to suspend precipitate in the 75% ethanol.

1.1.10 7500 g of solution is centrifuged at 4° C. for 5 min.

1.1.11 The supernatant is carefully pipetted and discarded, the solution is briefly centrifuged, and all the supernatant is pipetted and discarded. RNA precipitate is dried in a super clean bench for 5 min until ethanol is volatilized.

1.1.12 An appropriate amount of RNase-free ultra-pure water is added, and the precipitate is gently blown, so that the RNA is suspended in the ultra-pure water and the temperature is kept at 50° C. for 10 min. The RNA precipitate is completely dissolved and then stored at −70° C.

1.2 DNase I digest the RNA sample to remove any genomic DNA that may be contained therein. Preparation of a reaction solution: The reaction solution includes 20 μg of RNA, 10 μL of 10×DNase I buffer, 5 μL of DNase I, 1 μL of RNase inhibitor, and 100 μL of RNase-free water, with the total volume reaching 100 μL. The reaction solution is incubated at 37° C. for 30 min.

1.3 RNA purification: Purification is performed using RNeasy® MinElute™ purification kit (Qiagen) according to the instructions.

1.4 Quality inspection:

1.4.1 1 μL of total RNA sample is taken, and diluted by 20 times with RNase-free ultra-pure water, and UV quantification is performed.

1.4.2 1 μg of total RNA sample is taken, and 1.5% agarose gel electrophoresis analysis is performed to detect the mass of the total RNA sample.

1.5 Concentration determination: The reading value at 260 nm being 1 means 40 ng RNA/μL. The formula for calculating the RNA concentration of sample is: A₂₆₀×40 ng/μL.

1.6 Purity detection: The ratio of A₂₆₀/A₂₈₀ of the RNA solution is based on a method for detecting RNA purity, and the ratio ranges from 1.8 to 2.1.

1.7 Synthesis of cDNA

1.7.1 Preparation of a mixture: 1 μL of 500 ng/μL, oligo (dT)₁₈, 1.5 μg of total RNA, 1 μL of 10 mmol/L dNTP mixture and 13 μL of sterile water.

1.7.2 After water bath at 65° C. for 5 min, the mixture is placed on ice for standing for at least 1 min.

1.7.3 After brief centrifugation, 4 μL of 5×First-Strand Buffer, 1 μL of 0.1 mol/L DTT, 1 μL of RNase inhibitor and 1 μL of SuperScript. III RT are sequentially added to the centrifuge tube.

1.7.4 The mixture is pipetted gently by a pipette several times for uniform mixing.

1.7.5 The mixture is incubated at 50° C. for 60 min.

1.7.6 The mixture is incubated at 70° C. for 15 min to inactivate the enzyme.

1.7.7 91 μL of sterile water is added to every 20 μL of cDNA and uniformly mixed, and the mixture is placed in ice bath for later use or stored at −20° C.

1.8 Real-time quantitative PCR

1.8.1 Preparation of a mixture in a 5 mL tube or a multi-channel slot: 550 μL of 2×SuperArray PCR master mix (containing SYBR green fluorescent dye), 102 μL of diluted cDNA, and 448 μL of ddH₂O, with the total volume reaching 1100 μL.

1.8.2 Sample adding: An membrane on the PCR Array is opened, 10 μL of mixture is added to each well corresponding to the PCR Array, and a lid is closed to seal the PCR Array; after a real-time quantitative PCR program is set, the PCR Array is placed on the real-time quantitative PCR instrument for PCR reaction, and the prepared PCR Array is placed on ice before the PCR program is set.

1.9 Data analysis: A ΔΔCt method is adopted.

1.9.1 The ΔCt of genes in each treatment group is calculated:

ΔCt (test)=average Ct−average of housekeeping genes' Ct for test array

ΔCt (control)=average Ct−average of housekeeping genes' Ct for control array

1.9.2 The ΔΔCt of each gene in two groups of PCR Arrays is calculated: ΔΔCt=ΔCt (test)−ΔCt (control).

1.9.3 2^{-ΔΔCt} values of the test group and the control group are calculated to analyze the difference in expression of the corresponding genes between the test group and the control group. When the 2^{-ΔΔCt} value of the gene signal is greater than 2, it is determined that the expression of the gene in the test group is up-regulated; and when the 2^{-ΔΔCt} value is less than or equal to 2, it is determined that the expression of the gene in the test group is down-regulated. In addition, genes with large differences are selected for gene classification (GO) analysis, and signaling pathways that are mainly affected under the action of mPEG-SC₂₀ₖ-HM-3 are explored.

### 2. Flow Cytometry Test Method

### 2.1 Cell Treatment

Human U937 cells are added to a 6-well plate (1.5×10⁵ cells/mL), PMA (25 ng/mL) is added to perform induction for 48 h for differentiation into macrophages, and the macrophages are added to the positive drug Adalimumab group (50 μg/mL), the HM-3 control group (13.5 μmol/L), the mPEG-SC₂₀ₖ-HM-3 group (13.5 μmol/L), isovolumetric PBS is added to the blank group and the model group, and the cells are incubated in a CO₂ incubator at 37° C. for 24 h. For the H₂O₂ group (3%, 20 μL/mL volume) test, hydrogen peroxide is added in the first half hour to normal cells for apoptosis induction control.

### 2.2 Cell Collection

1000 g of solution is centrifuged for 5 min, and supernatant is discarded. Washing is performed once with PBS.

### 2.3 Macrophage Apoptosis Test by Double Staining With a Flow Cytometer

The apoptosis is tested on the flow cytometer according to the instructions of the AV-FITC/PI apoptosis kit.

### 3. Western Blot Test Method

3.1 U937 cells are added to a 6-well plate (1.5×10⁵ cells/mL), PMA (25 ng/mL) is used for induction for 48 h, and the cells adhere and differentiate into macrophages. Positive drug Adalimumab (25 μg/mL, 50 μg/mL, 100 μg/mL, 200 μg/mL, 400 μg/mL), Avastin (25 μg/mL, 50 μg/mL) are added, the final concentrations of mPEG-SC₂₀ₖ-HM-3 are 2.25, 4.5, 9.0, 13.5, 18.0, 36 μmol/L, and isovolumetric PBS is added to the blank group and the model group. The cells are incubated in a CO₂ incubator at 37° C. for 24 or 48 h. Rat synovial cells are induced with LPS for 24 h and then mPEG-SC₂₀ₖ-HM-3 is administrated to final concentrations of 2.25, 4.5, 9.0, 13.5, 18.0 μmol/L, and the cells are incubated in a CO₂ incubator at 37° C. for 48 h.

3.2 After the third generation of human umbilical vein endothelial cells (HUVECs) are cultured in a 6-well plate to more than 80% fusion, the polypeptide mPEG-SC₂₀ₖ-HM-3 is added to final concentrations of 0.5, 1.0, 2.0, 4.0, 8.0 μmol/L, and PBS is used as a blank control. The cells are incubated in a CO₂ incubator at 37° C. for 24 h; the cells are digested with 0.25% trypsin and counted, the solution is centrifuged at 3000 rpm for 5 min, and the cells are collected.

3.3 A protein extract solution is added at 20 μL/1×10⁵ cells, and the cells are pipetted and dispersed, and stand at 4° C. for 30 min to lyse the cells.

3.4 1/4 volume of 5×protein loading buffer is added, and boiling water bath is performed for 5-10 min.

3.5 20 μL of protein sample is taken and 10% SDS-PAGE electrophoresis is performed.

3.6 The protein on the gel is transferred to the PVDF membrane by using a wetting transfer method with a constant current of 100 mA for 1-2.5 h.

3.7 The PVDF membrane is blocked with a blocking solution at room temperature for 2 h.

3.8 The membrane is washed for 3 times with PBST, 5 min each time. A primary antibody is added and the membrane stands at 4° C. overnight or is incubated at 37° C. for 2 h.

3.9 The membrane is washed for 3 times with PBST, 5 min each time. A secondary antibody is added and the membrane is incubated at room temperature for more than 2 h.

3.10 The membrane is exposed in a gel imager using an ECL detection kit.

### Experimental Results

### 1. Test Results for a Human Whole Genome Array

### 1.1 Extraction of Total RNA

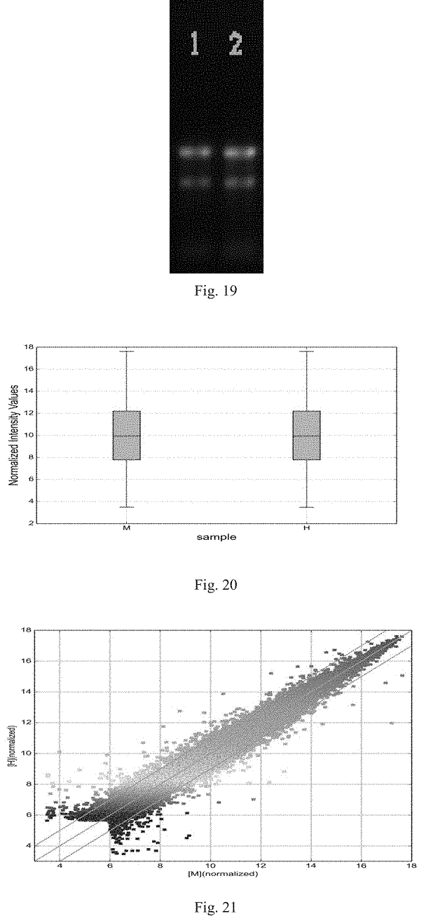

The extracted total RNA of cells is purified and detected, the concentration of RNA in experimental group (administration) is 1300.40 ng/μL, and A₂₆₀/A₂₈₀ is 2.02; the concentration of RNA in the control group (negative) is 869.47 ng/μL, and A₂₆₀/A₂₈₀ is 2.02. Agarose gel electrophoresis indicates that bands of 28S and 18S ribosomal RNA are very bright and dense (the size depends on the species used to extract RNA), and the density of the upper band is about twice that of the lower band. There is no DNA pollution or degradation during RNA preparation. The results show that the purity of total RNA is high (FIG. 19), which proves that the integrity of the extracted total RNA is good and meets the requirements for the next step of detection.

### 1.2 Gene Intensity Box-Plot

The human whole genome array (Human 4×44 K Gene Oligo Microarray Kit) from Agilent Technologies, Inc. from USA is used to analyze the gene expression profile. A block diagram is used as a convenient visual database distribution map to compare the intensity distribution of all samples. After normalization, the log2-ratios distributions of all samples are almost the same (FIG. 20).

### 1.3 Scatter Plot of Differentially Expressed Genes

The visualized scatter plot is used to compare and analyze the difference in gene expression of samples. In the scatter plot, the X-axis and Y-axis are the normal values of the samples (log2 scaly points). The green line represents the multiple change boundary line, and the multiple change value beyond the boundary line is 2.0. In the sample comparison results, the points above the green line and below the green line are points where the gene expression multiples are more than 2.0 times (FIG. 21).

### 1.4 Heat Map and Hierarchical Clustering of Differential Genes

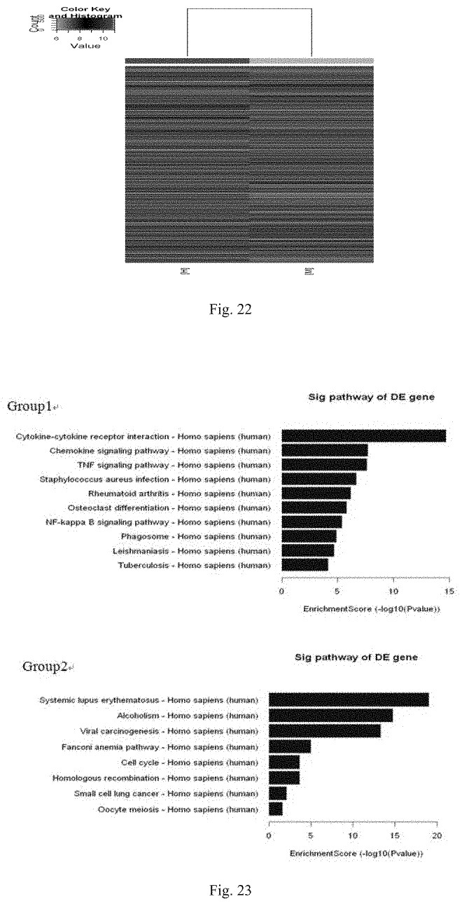

Hierarchical clustering analysis arranges the gene expression levels in the two groups and can infer the correlation between the two groups of samples. Clustering analysis mainly focuses on the values of all targets. Red indicates high correlation expression, and blue indicates low correlation expression (FIG. 22).

### 1.5 Pathway Analysis of Differential Genes

Pathway analysis is based on a diagram of functional gene KEGG pathway analysis. P value (EASE-score, Fisher-Pvalue or Hypergeometric-Pvalue) indicates the significance of condition-related pathways. A lower P value indicates a more important pathway (the recommended value is less than 0.05). The following figure shows the comparison between the negative group and the test group, showing signaling pathway up-regulation situations and signaling pathway down-regulation situations (FIG. 23).

### 1.5 Bioinformatics Analysis of Differential Genes

The human whole genome array is used to detect the changes in the expression abundance of related factors mRNA levels in the cells after mPEG-SC₂₀ₖ-HM-3 acts on human U937 cells. The results show that after mPEG-SC₂₀ₖ-HM-3 acts on the human U937 cells, the expression of 499 related factors is up-regulated at the mRNA level, and the expression of 538 related factors is down-regulated at the mRNA level, covering a comprehensive range.

By gene classification (GO) analysis of the foregoing results, genes related to RA inflammation are selected. The up-regulated ones are: inflammation-related (IL-1RN, IL-10, IL22RA2) and apoptosis-related (TRAF1, TNFRSF1B, TNFRSF9, TNFRSF11A, TNFSF13\1415); and the down-regulated ones are: cell adhesion, migration-related (PODXL), cell proliferation, differentiation-related (CENPQ, NRARP, HIRA, FGFBP3), chemokines (CXCL1\5, CCL3\20), inflammation-related (IL-1A, COX18, BCL2\11A, BCX, MVB12B, TRAIP, HEST), and rheumatoid-related (FGFBP3, OMD).

Up-regulated genes related to inflammation indicate that: mPEG-SC₂₀ₖ-HM-3 mainly acts on the activation function of a variety of cell lines, inhibits the production of cytokines by activated T cells, and especially inhibits the production of IL-2/IFN-γ cells and other cells by Th1 cells, thereby regulating cellular immune response and antigen presentation ability; inhibit TNF signaling pathway, and promote cell apoptosis, etc. Down-regulated genes indicate that mPEG-SC₂₀ₖ-HM-3 inhibits cell adhesion and migration, inhibits cell motility, inhibits Notch regulated ankyrin, inhibits cell differentiation and proliferation, inhibits production and proliferation of antigen-specific antibodies, and participates in angiogenesis, inflammation, wound healing and tumorigenesis and is combined to be fibroblast growth factors.

By gene classification (GO) analysis of the foregoing results, genes related to RA inflammatory angiogenesis are selected. The up-regulated ones are: cell matrix (MMP3\7\8\10\12\19), and cell metabolism (MTSS1, AMICA1, GALM), and the down-regulated ones are: cytoskeleton (CKAP2L), cell adhesion-related (RASGRP2), angiogenesis-related (ECE1, PTPRU, THBS4, AMOT, VEGFE, PDGF), cell senescence-related (MTFR2), and metabolism-related (LRP3).

Up-regulated genes related to angiogenesis indicate that mPEG-SC₂₀ₖ-HM-3 promotes the regulation of cellular stromelysins and metabolic regulation pathways, and reduces angiogenesis caused by hypoxia. Down-regulated genes indicate that mPEG-SC₂₀ₖ-HM-3 inhibits cytoskeleton and motility and migration, and decreases the functions of participating in embryo formation, neovascularization and wound healing. In the pathological state, it inhibits the pathological process of rheumatoid arthritis, malignant tumor metastasis, atherosclerosis (AS) and acute coronary syndrome (ACS), and oxidative aging.

### 2. Flow Cytometry Apoptosis Test Results

Results show (FIG. 24) that the Adalimumab positive control group promotes early and late apoptosis of macrophages at rates of 14.2% and 15.2%, respectively; and mPEG-SC₂₀ₖ-HM-3 promotes early and late apoptosis of macrophages at rates of 28.0% and 14.7% respectively. mPEG-SC₂₀ₖ-HM-3 mainly promotes the early apoptosis of macrophages, and mPEG-SC₂₀ₖ-HM-3 promotes early apoptosis of macrophages better than Adalimumab.

### 3. Western Blot Test Results

HUVECs cells can be directly detected without establishing an induction model. However, the process of inducing human U937 monocytes to become macrophages leads to the up-regulation of TLR-4, IKKs and NF-κB protein expression in the downstream of the Tolls receptor, which makes the transcription factor NF-kB free into the nucleus, starts the transcription and expression of downstream inflammatory factors (HIF-1β, TNF-α) and angiogenesis (IL-6, VEGF) protein factors, and stimulates the up-regulation of TNFR1 and TNFR2 expression, thus triggering the inflammatory cycle activation and promoting angiogenesis.

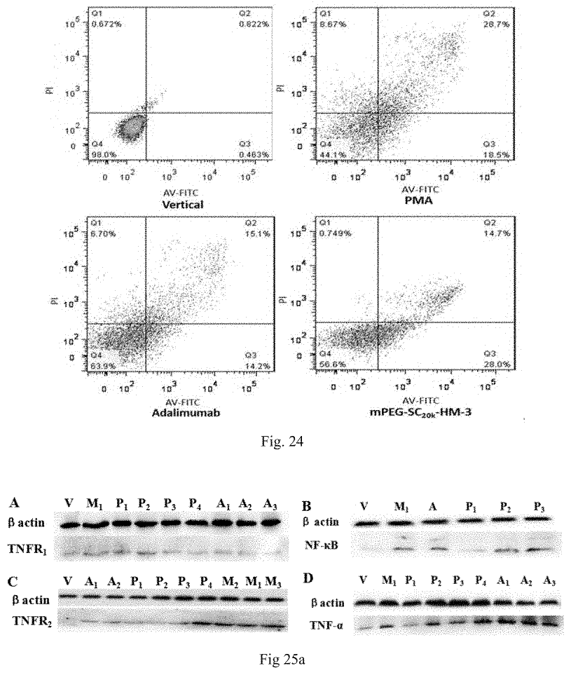

The results of Western blot show that (FIG. 25a and FIG. 25b), under the action of mPEG-SC₂₀ₖ-HM-3, with the increase of the dosage of mPEG-SC₂₀ₖ-HM-3, the expression of TNFR1 and TNFR2 protein factors in macrophages is down-regulated, and the expression of NF-κB protein factors in nuclei is down-regulated, which inhibits the expression of TNF-α protein after inhibiting its nuclear transcription. Compared with positive drug Adalimumab, mPEG-SC₂₀ₖ-HM-3 has the best inhibitory effect on the expression of TNFR2, NF-κB and TNF-α proteins at a small dose (4.5 μM), and the best inhibitory effect on the expression of TNFR1 protein at a large dose (18 μM). Therefore, the regulatory effect of mPEG-SC₂₀ₖ-HM-3 on pro-apoptotic pathway-related proteins and its sphere of influence on protein expression are better than those of Adalimumab.

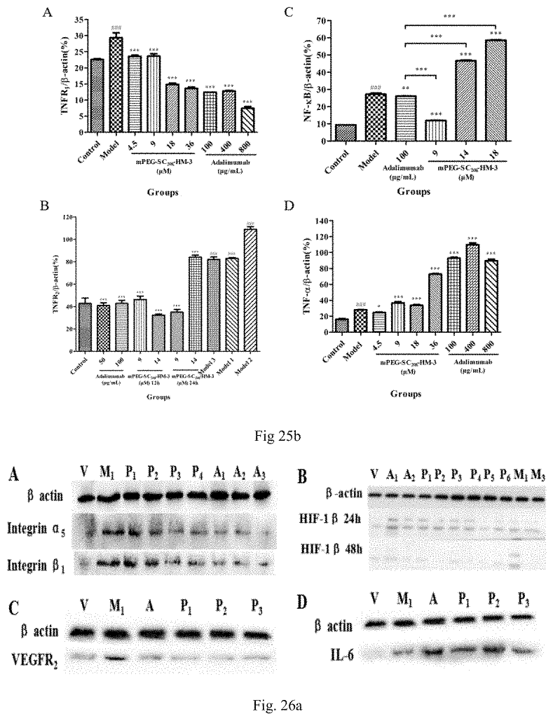

The results of Western blot show that (FIG. 26a and FIG. 26b), under the action of mPEG-SC₂₀ₖ-HM-3, with the increase of the dosage of mPEG-SC₂₀ₖ-HM-3, The expression of integrin α5, Integrin β1 and VEGFR2 proteins in macrophages is down-regulated, and the expression of HIF-1β and IL-6 protein in macrophages is down-regulated. Compared with positive drug Adalimumab, mPEG-SC₂₀ₖ-HM-3 has the best inhibitory effect on the expression of Integrin α5, Integrin β1, VEGFR2, HIF-1β, IL-6 proteins at a dose of 13.5 μM. Therefore, the effect of mPEG-SC₂₀ₖ-HM-3 on the regulation of integrin pathway angiogenesis is better than that of Adalimumab, and Adalimumab has no inhibitory effect on integrin pathway angiogenesis.

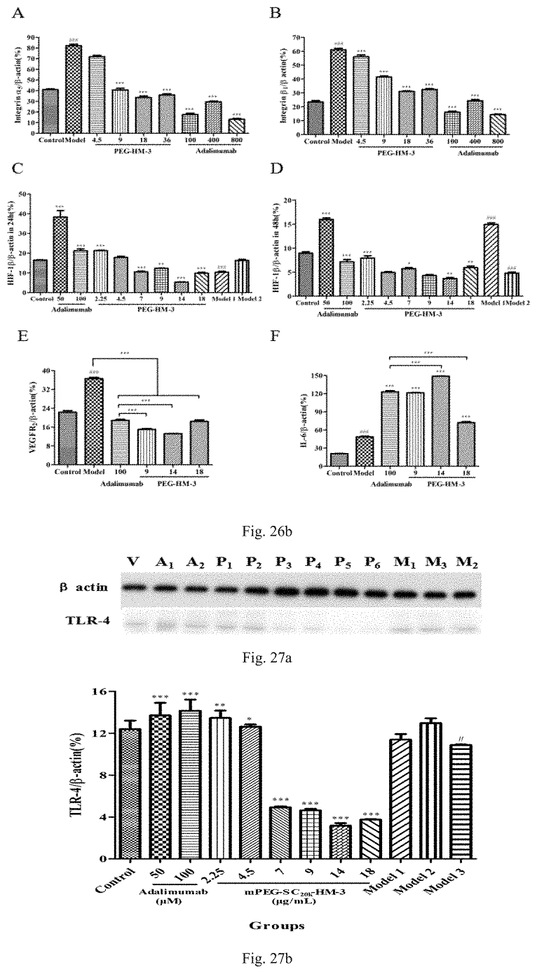

Western blot test results show that (FIG. 27a and FIG. 27b), with the increase of mPEG-SC₂₀ₖ-HM-3 dosage in rat synovial cells, the down-regulation of the expression of TLR-4 protein presents a certain linear trend. Compared with positive drug Adalimumab, mPEG-SC₂₀ₖ-HM-3 has the best inhibitory effect on the expression of TLR-4 protein at a dose of 13.5 μM. Therefore, the anti-inflammatory effect of mPEG-SC₂₀ₖ-HM-3 on the inhibition of TLR4 protein signaling pathway and downstream NF-κB expression is better than that of Adalimumab.

In conclusion, in macrophages, mPEG-SC₂₀ₖ-HM-3 inhibits the nuclear NF-κB transcription function by inhibiting integrin ανβ3 pathway, and down-regulates the downstream HIF-1β and IL-6 expression. mPEG-SC₂₀ₖ-HM-3 down-regulates the downstream TNF-α expression by inhibiting TLRs signaling pathway and TNFR1\2 apoptosis signaling pathway, and at the same time promotes macrophage apoptosis, so as to exert the inflammation inhibiting effect.

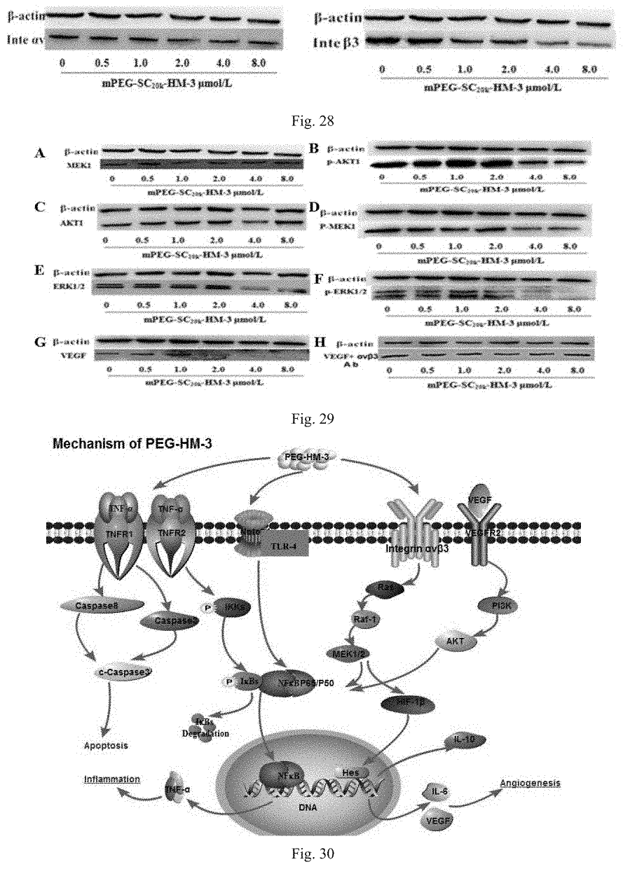

In HUVECs cells, under the action of mPEG-SC₂₀ₖ-HM-3, the expression of integrin αν, integrin β3, MEK1, p-MEK1, PI3K, AKT1, p-AKT1, ERK1/2 and p-ERK1/2 is down-regulated, and the expression of VEGF is down-regulated, presenting a certain dose dependence. It indicates that mPEG-SC₂₀ₖ-HM-3 inhibits angiogenesis mainly by inhibiting the expression of downstream protein molecules through integrin ανβ3 pathway (FIG. 28 and (FIG. 29).

Therefore, by binding to integrin ανβ3, mPEG-SC₂₀ₖ-HM-3 inhibits the downstream pathway of integrin pathway, and finally inhibits NF-kB protein and its intranuclear transcription activity, so as to inhibit the expression of pro-inflammatory factors (HIF-1β, TNF-α, IL-6) and inflammation-related receptor proteins (TLR-4, TNFR1\2), thus achieving the effect of inhibiting inflammation and promoting apoptosis; and at the same time, it inhibits the transcription and expression of VEGF and its receptor in cells, and achieves the goal of inhibiting angiogenesis (FIG. 30).

In this test, a whole genome sequencing analysis technology is used to test gene changes of human macrophages under the action of mPEG-SC₂₀ₖ-HM-3. Through whole genome analysis, one-time analysis and comparison of differential gene expression abundance features comprehensiveness, directivity and good screening. It is convenient for us to grasp the overall difference, and select unknown polypeptide targets and signaling pathways, which facilitates in-depth study of drug mechanisms in the future. In this test, the array analysis results show that after mPEG-SC₂₀ₖ-HM-3 acts on macrophages, the expression of 499 related factors is up-regulated at the mRNA level, and the expression of 538 related factors is down-regulated at the mRNA level, covering a comprehensive range. Genes related to RA inflammation are selected. The up-regulated ones are: inflammation-related (IL-1RN, IL-10, IL22RA2) and apoptosis-related (TRAF1, TNFRSF1B, TNFRSF9, TNFRSF11A, TNFSF13\1415); and the down-regulated ones are: cell adhesion, metastasis-related (PODXL), cell proliferation, differentiation-related (CENPQ, NRARP, HIRA, FGFBP3), chemokines (CXCL1\5, CCL3\20), inflammation-related (IL-1A, COX18, BCL2\11A, BCX, MVB12B, TRAIP, HEST), and rheumatoid-related (FGFBP3, OMD). Up-regulated genes related to inflammation present that: mPEG-SC₂₀ₖ-HM-3 mainly acts on the activation function of a variety of cell lines, inhibits the production of cytokines by activated T cells, and especially inhibits the production of IL-2\IFN-γ cells and other cells by Th1 cells, thereby regulating cellular immune response and antigen presentation ability; inhibit TNF signaling pathway, and promote cell apoptosis, etc. Down-regulated genes indicate that mPEG-SC₂₀ₖ-HM-3 inhibits cell adhesion and migration, inhibits cell motility, inhibits Notch regulated ankyrin, inhibits cell differentiation and proliferation, inhibits production and proliferation of antigen-specific antibodies, and participates in angiogenesis, inflammation, wound healing and tumorigenesis and is combined to be fibroblast growth factors.

Genes related to RA inflammatory angiogenesis are selected. The up-regulated ones are: cell matrix (MMP3\7\8\10\12\19), and cell metabolism (MTSS1, AMICA1, GALM), and the down-regulated ones are: cytoskeleton (CKAP2L), cell adhesion-related (RASGRP2), angiogenesis-related (ECE1, PTPRU, THBS4, AMOT, VEGFE, PDGF), cell senescence-related (MTFR2), and metabolism-related (LRP3). Up-regulated genes related to angiogenesis indicate that mPEG-SC₂₀ₖ-HM-3 promotes the regulation of cellular stromelysins and metabolic regulation pathways, and reduces angiogenesis caused by hypoxia. Down-regulated genes indicate that mPEG-SC₂₀ₖ-HM-3 inhibits cytoskeleton and motility and migration, and decreases the functions of participating in embryo formation, neovascularization and wound healing. In the pathological state, it inhibits the pathological process of rheumatoid arthritis, malignant tumor metastasis, atherosclerosis (AS) and acute coronary syndrome (ACS), and oxidative aging. Therefore, the array indicates that the main function of mPEG-SC20k-HM-3 is not only related to inflammation regulation and cell differentiation, but also related to angiogenesis regulation.

The flow cytometry apoptosis results show that mPEG-SC₂₀ₖ-HM-3 promotes early and late apoptosis of macrophages at rates of 28.0% and 14.7%, respectively, which is better than that of the positive drug Adalimumab group at rates of 14.2% and 15.2%. Therefore, mPEG-SC₂₀ₖ-HM-3 functions to inhibit and relieve inflammation by inhibiting early activity of macrophages and promoting the apoptosis of macrophages. The Western blot test results show that mPEG-SC₂₀ₖ-HM-3 mainly inhibits integrin ανβ3 and its downstream pathway, and finally inhibits NF-kB protein and its intranuclear transcription activity, so as to inhibit the expression of pro-inflammatory factors (HIF-1β, TNF-α, IL-6) and inflammation-related receptor proteins (TLR-4, TNFR1\2), and to inhibit inflammation. mPEG-SC₂₀ₖ-HM-3 down-regulates the expression of integrin αν, integrin β(3, MEK1, p-MEK1, PI3K, AKT1, p-AKT1, ERK1/2 and p-ERK1/2 protein factors in HUVEC cells, and down-regulates the expression of VEGF, IL-6 and HIF-1β in cells.

Therefore, by binding to integrin ανβ3, mPEG-SC₂₀ₖ-HM-3 inhibits the downstream pathway of integrin pathway, and finally inhibits NF-kB protein and its intranuclear transcription activity, so as to inhibit the expression of pro-inflammatory factors (HIF-1β, TNF-α, IL-6) and inflammation-related receptor proteins (TLR-4, TNFR1\2), thus achieving the effect of inhibiting inflammation and promoting apoptosis; and at the same time, it inhibits the transcription and expression of VEGF and its receptor in cells, and achieves the goal of inhibiting angiogenesis.

### Screening of PEG Modification

There are many ways to modify polypeptides by PEG, such as modification by amino, modification by carboxyl and modification by sulfhydryl. There are many kinds of PEG derivatives (mPEGs) used for modification by amino, which can be divided into two categories: acylated mPEGs and alkylated mPEGs. The acylated mPEGs include mPEG-succinimide ester (mPEG-SC) and mPEG-benzotriazole carbonate, mPEG- nitrobenzene carbonate, mPEG-trichlorobenzene carbonate and mPEG-oxycarbonyl imidazole, etc., and the alkylated mPEGs include mPEG-aldehyde, mPEG-trifluoroethanesulfonic acid and mPEG-epoxide, etc. For the polypeptides of the present invention, after preliminary experimental selection, it is finally determined to perform modification by amino. The PEG derivatives are selected mPEG-ALD (monomethoxypolyethylene glycol propionaldehyde) and mPEG-SC (monomethoxy polyethylene glycol succinimide carbonate), which are two different types of amino modifiers.

Four different types of mPEG-ALD and mPEG-SC with different molecular weights are selected to modify the polypeptide Ile-Val-Arg-Arg-Ala-Asp-Arg-Ala-Ala-Val-Pro-Gly-Gly-Gly-Gly-Arg-Gly-Asp (named HM-3), i.e., mPEG-ALD₅ₖ, mPEG-ALD₁₀ₖ, mPEG-SC₁₀ₖ, mPEG-SC₂₀ₖ, and modification reaction conditions are explored and optimized. The activity of four PEG-modified polypeptides is selected by pharmacodynamic evaluation in BALB/c nude mice to obtain PEG-modified drugs with better activity.

(1) mPEG-ALD₅ₖ-Modified HM-3

The molecular formula of HM-3 is C₇₂H₁₂₆N₃₀O₂₃, and its terminal amino group can be coupled to mPEG, with the reaction formula as follows:

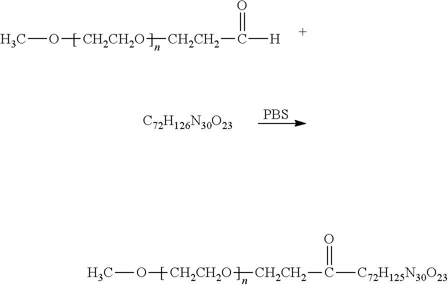

(2) mPEG-ALD₁₀ₖ-Modified HM-3

The reaction formula is:

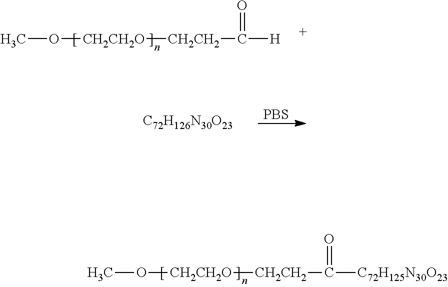

(3) Preparation of mPEG-SC₁₀ₖ-Modified HM-3

The reaction formula is:

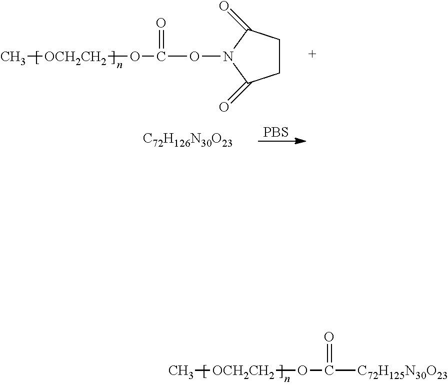

(4) Preparation of mPEG-SC₂₀ₖ-Modified HM-3

The reaction formula is:

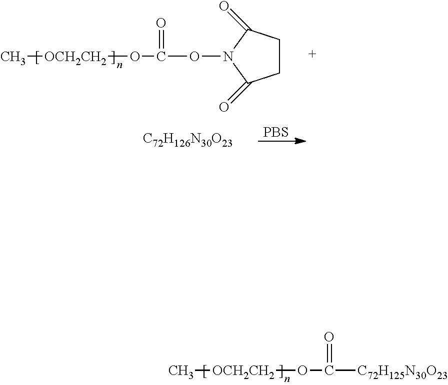

### (5) Conditions for Modification by PEG

Through preliminary exploration in the early stage, four reaction conditions of PEG-modified HM-3 were established as shown in the following table.

TABLE 1-1
Conditions for PEG-modified HM-3

| pH of PBS | | Molar ratio | Modification |
|---|---|---|---|
| buffer | PEG | (PEG:HM-3) | rate |
| PBS 5.0 | ALD-mPEG₅ₖ | 2:1 | 69.61% |
| PBS 5.0 | ALD-mPEG₁₀ₖ | 2.5:1 | 88.41% |
| PBS 8.0 | SC-mPEG₁₀ₖ | 1.5:1 | 95.94% |
| PBS 8.5 | SC-mPEG₂₀ₖ | 1.5:1 | 96.84% |

### (6) Activity Screening of PEG-Modified HM-3.

The four modified products, mPEG-ALD₅ₖ-HM-3, mPEG-ALD₁₀ₖ-HM-3, mPEG-SC₁₀ₖ-HM-3 and mPEG-SC₂₀ₖ-HM-3, are evaluated by in vivo pharmacodynamics of BALB/c nude mice, and the most ideal modified product mPEG-SC₂₀ₖ-HM-3 is selected. mPEG-SC₂₀ₖ-HM-3 better retains the original biological activity of HM-3, and its antitumor effect in vivo is better than that of paclitaxel, reaching 50.23% (44.50% for the paclitaxel, and 43.92% for 1-1M-3). At the same time, its elimination half life in vivo has also been greatly improved, and its administration frequency has been decreased from twice a day to once every two days. During the administration, the body weight of nude mice in each group remains basically stable, and no group has drastic changes, indicating that the modified drug has very low toxicity or no toxicity at an effective dose in vivo.

### Embodiment 1

The solid-phase synthesis is adopted to synthesize polypeptide I, polypeptide II and polypeptide III. The synthesized products are purified with the high performance liquid chromatography (HPLC), and then their purity is determined by reversed phase high performance liquid chromatography (RP-HPLC). The synthesis method disclosed in prior patents, namely ZL 201110194918.0 and ZL201110370529.9, is adopted in the present invention.

RESULT: RP-HPLC analysis shows that the purity of the synthesized polypeptides I, polypeptide II and polypeptide III is 96.94%, 99.30%, 96.34% respectively; the result meets the required purity standard.

### Embodiment 2

In Vivo Immunoprotective Effect of polypeptide I on CIA Mouse Models

Investigating the therapeutic effect of polypeptides disclosed in the present invention on mouse collagen-induced arthritis (CIA) by means of establishing CIA mouse models. Taking 60 specific, pathogen-free DBA/1 mice (provided by Sino-British SIPPR/BK Lab. Animal Ltd, Shanghai, China; animal production license: SCXK (Shanghai) 2008-0016) as animal subjects, randomly dividing 7- or 8-week-old male mice with body weight of 18-22 g into 6 groups, namely, the normal control group, model control group, polypeptide I groups including the low-dose (0.2 mg/kg), medium-dose (0.4 mg/kg) and high-dose (0.8 mg/kg) subgroups and the (methotrexate 1 mg/kg) positive control group. Apart from the normal control group, CIA mouse models are established for all test groups on day 0: Predissolving chicken cartilage collagen type II (cII) into 4 mg/ml solution in 0.1 mol/L acetic acid, and then keeping the solution in a refrigerator at 4° C. overnight. On day 0 of the experiment, sufficiently emulsifying type II collagen solution with isovolumetric complete Freund's adjuvant (CFA) containing 4 mg/ml Mycobacterium tuberculosis (strain H37Rv); anesthetizing DBA/1 mice and intradermically injecting 50 μl emulsion at the tail of each mouse; on day 21 of the experiment, inducing the secondary immune response by intradermically injecting 50 μl emulsion at the tail of each mouse again; the emulsion used for said secondary immune response is prepared by sufficiently emulsifying 4 mg/ml type II collagen (cII) and isovolumetric incomplete Freund's adjuvant (IFA). On day 30 of the experiment, hypodermically administering drugs for each mouse; polypeptide I is further divided into low-dose (0.2 mg/kg), medium-dose (0.4 mg/kg) and high-dose ((0.8 mg/kg), twice a day, 10 days in succession; and mice from the positive control group are administered with methotrexate (1 mg/kg), once every 5 days, three times in total; mice from the normal control group and model control group are administered only with physiological saline on daily basis, 10 days in succession. During day 21 to day 70 of the experiment, evaluating the effect of the drugs on CIA mouse models by measuring the body weight, scoring the joint change and examining the diameter of left and right hind ankles, once every 3 days. On day 70 of the experiment, killing mice with cervical dislocation.

The arthritis is evaluated in accordance with the following criteria:

### 1) Joint Scoring

Four legs: scoring in terms of level 0 to level 4, 5 levels in total. Specifically: 0=no red spot or swelling; 1=small red spot or slight swelling appeared at one of front/hind toe joints); 2=red spot or swelling appeared at more than one front/hind toe joints; 3=paw swelling beneath ankles; 4=paw swelling including ankles. Four feet are scored independently, with 16 as the highest point.

Scoring are conducted during day 21 to day 70 of the experiment, once every 3 days, and recording all the data.

### 2) Measuring the Diameter of Mouse Ankles

Measuring the diameter of both left and right ankles (inside-outside) and the thickness of paws of mice with a vernier caliper before the model establishment and during day 21 to day 70 of the experiment, once every 3 days; recording all the data.

The measured data are listed in the form of mean and standard deviation (mean±SD), conducting T-test with SPSS 11.0 software for all test groups and control groups, wherein * refers to p<0.05 and **p<0.01.

RESULTS: comparing the model mice with the normal mice. On day 0 of the experiment, the model mice are firstly hypodermically injected at the tail with an emulsion made by collagen and isovolumetric CFA (containing deactivated Mycobacterium tuberculosis); on day 21 of the experiment, the model mice are again hypodermically injected at the tail with an emulsion made by collagen and isovolumetric IFA; on day 27 of the experiment, swelling at paws appears on CIA mice and points for arthritis scoring start increasing; the highest degree of swelling on model mice appeared on day 45-60; in addition, the body weight of model mice stops increasing since day 35 and even slightly decreases later on. The in vivo immunoprotective effect of polypeptide I on CIA mouse models is shown in table 1. As is shown in the table, all doses of polypeptide I present immunoprotective effect on CIA mouse models:

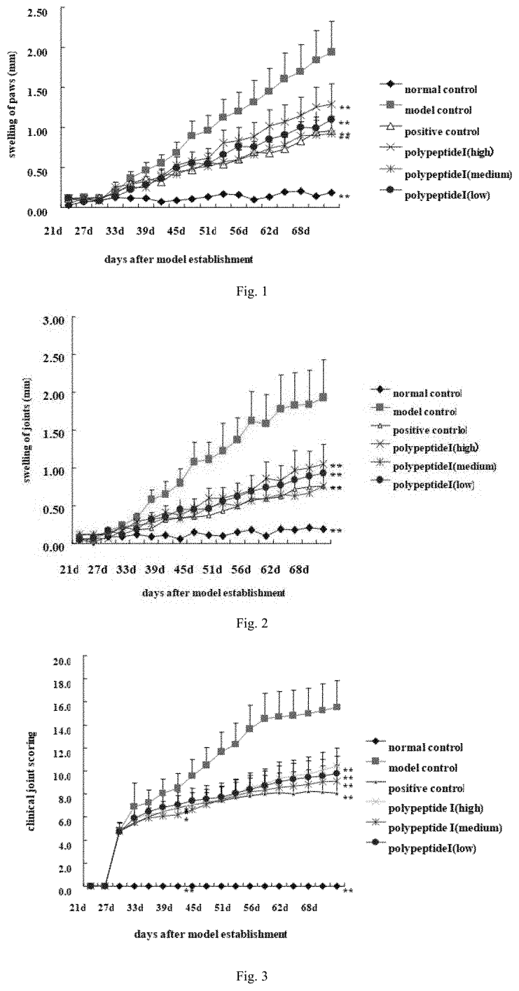

The effect of polypeptide I in improving paw swelling of CIA mouse models is demonstrated in FIG. 1. Insofar as the degree of paw swelling is concerned, both the positive control group and the polypeptide I group (including high, medium and low doses) present extremely significant difference (p<0.01) in contrast with the model control group; the test result is statistically significant. The effect of polypeptide I in improving joint swelling of CIA mouse models is demonstrated in FIG. 2. Insofar as the degree of joint swelling is concerned, both the positive control group and the polypeptide I group (including high, medium and low doses) present extremely significant difference (p<0.01) in contrast with the model control group; the test result is statistically significant. The effect of polypeptide I in improving joint scoring of CIA mouse model is demonstrated in FIG. 3. The scoring points of Polypeptide I group (including high, medium and low doses) are greatly lower than that of the control group; there exists extremely significant difference (p<0.01) between these two groups, and the test result is statistically significant.

TABLE 1
in vivo immunoprotective effect of polypeptide I on
CIA mouse models

| | num- | dose | | | |
|---|---|---|---|---|---|
| | ber | (mg/ | swelling of | swelling of | clinical |
| group | (n) | kg) | paws (mm) | joints (mm) | scoring |
| normal | 10 | — | 0.18 ± 0.05** | 0.19 ± 0.04** | 0.00 ± 0.00** |
| control | | | | | |
| normal | 10 | — | 1.94 ± 0.37 | 1.93 ± 0.40 | 15.5 ± 2.3 |
| control | | | | | |
| positive | 10 | 1 | 0.95 ± 0.19** | 0.76 ± 0.17** | 8.1 ± 1.2** |
| control | | | | | |
| polypeptide | 10 | 0.8 | 1.29 ± 0.26** | 1.05 ± 0.31** | 10.4 ± 1.6** |
| I(high) | | | | | |
| polypeptide | 10 | 0.4 | 0.92 ± 0.18** | 0.76 ± 0.17** | 9.1 ± 1.4** |
| I(medium) | | | | | |
| polypeptide | 10 | 0.2 | 1.10 ± 0.22** | 0.93 ± 0.23** | 9.7 ± 1.5** |
| I(low) | | | | | |

*referring to p < 0.05,
**referring to p < 0.01.
CONCLUSION: polypeptide I has therapeutic effect on collagen-induced arthritis in mice

### Embodiment 3

### In Vivo Immunoprotective Effect of Polypeptide I on AIA Rat Models

Investigating the therapeutic effect of polypeptides disclosed in the present invention on adjuvant-induced arthritis (AIA) in rats by means of establishing AIA rat models. Taking specific, pathogen-free SD rats (provided by Sino-British SIPPR/BK Lab Animal Ltd, Shanghai, China; animal production license: SCXK (Shanghai) 2008-0016) as animal subjects, randomly dividing 60 male rats with body weight of 140-160 g into 6 groups, namely, the normal control group, model control group, polypeptide I groups including the low-dose (0.1 mg/kg), medium-dose (0.2 mg/kg) and high-dose (0.4 mg/kg) subgroups and the (methotrexate 1 mg/kg) positive control group. Apart from the normal control group, AIA rat models are established for all test groups on day 0 by injecting at the left hind paw of all rats with 0.08 ml CFA containing 10 mg/ml deactivated Mycobacterium tuberculosis (strain H37RA). On day 10 of the experiment, hypodermically administering drugs for each rat; polypeptide I is further divided into low-dose (0.1 mg/kg), medium-dose (0.2 mg/kg) and high-dose (0.4 mg/kg), twice a day, 10 days in succession; and mice from the positive control group are administered with methotrexate (1 mg/kg), once every 5 days, three times in total; rats from the normal control group and model control group are administered only with physiological saline on daily basis, 10 days in succession. On day 8, 11, 14, 17, 20, 23 and 26 of the experiment, evaluating the effect of the drugs on AIA rat models by examining the diameter of both left and right hind ankles.

The arthritis is evaluated in accordance with the following criteria:

### 1) Joint Scoring

Four legs: scoring in terms of level 0 to level 4, 5 levels in total. Specifically: 0=no red spot or swelling; 1=small red spot or slight swelling appeared at one of front/hind toe joints); 2=red spot or swelling appeared at more than one front/hind toe joints; 3=paw swelling beneath ankles; 4=paw swelling including ankles. Four feet are scored independently, with 16 as the highest point.

Scoring joints on day 8, 11, 14, 17, 20, 23 and 26 of the experiment and recording all the data.

### 2) Measuring the Diameter of Rat Ankles

Measuring the diameter of both left and right ankles (inside-outside) and the thickness of paws of rats with a vernier caliper before the model establishment and during day 21 to day 70 of the experiment, once every 3 days; recording all the data.

The measured data are listed in the form of mean and standard deviation (mean±SD), conducting T-test with SPSS 11.0 software for all test groups and control groups, wherein * refers to p<0. 05 and **p<0.01

RESULTS: comparing the model rats with the normal rats. The primary arthritis appears at the left hind paw of model rats soon after injection of CFA containing deactivated Mycobacterium tuberculosis at the left hind paw, along with apparent swelling and ulceration; the secondary arthritis appears at the right hind paw about 10 days later, with increasingly high scores; meanwhile, apparent angiogenesis occurs at rat ears, with obvious redness and swelling; swelling also appears at tail joints. The in vivo immunoprotective effect of polypeptide I on AIA rat models is shown in table 2. As is shown in the table, all doses of polypeptide I present immunoprotective effect on AIA rat models:

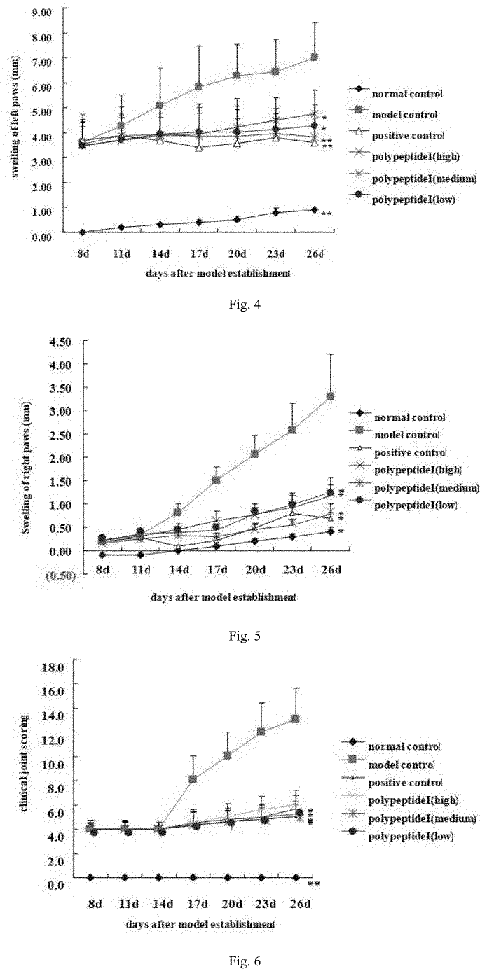

The effect of polypeptide I in improving the swelling degree of left paws induced by primary AIA is shown in FIG. 4. Insofar as the diameter of the left hind ankles is concerned, both the positive control group and polypeptide I medium-dose group present extremely significant difference (p<0.01) in contrast with the model control group, whereas both polypeptide I low-dose group and polypeptide I high-dose group present significant difference (p<0.05) in contrast with the model control group; the test result is statistically significant. The effect of polypeptide I in improving the swelling degree of right paws induced by secondary AIA is shown in FIG. 5. Insofar as the diameter of the right hind ankles is concerned, both the positive control group and the polypeptide I group (including low, medium and high doses) present significant difference (p<0.05) in contrast with the model control group. The effect of polypeptide I in improving joint scoring of AIA rat models is demonstrated in FIG. 6. The scoring points of polypeptide I group (including high, medium and low doses) are greatly lower than that of the control group; there exists significant difference (p<0.05) between these two groups, and the test result is statistically significant.

TABLE 2
in vivo immunoprotective effect of polypeptide I on AIA rat models

| | num- | dose | swelling of | swelling of | |
|---|---|---|---|---|---|
| | ber | (mg/ | left paw | right paw | clinical |
| group | (n) | kg) | (mm) | (mm) | scoring |
| normal | 10 | — | 0.90 ± 0.18** | 0.40 ± 0.08** | 0.0 ± 0.0** |
| control | | | | | |
| model | 10 | — | 7.01 ± 1.4 | 3.29 ± 0.94 | 13.1 ± 2.6 |
| control | | | | | |
| positive | 10 | 1 | 3.60 ± 0.72** | 0.68 ± 0.19** | 5.0 ± 1.0* |
| control | | | | | |
| polypeptide | 10 | 0.4 | 4.77 ± 0.95* | 1.25 ± 0.30* | 6.0 ± 1.2* |
| I(high) | | | | | |
| polypeptide | 10 | 0.2 | 3.81 ± 0.76** | 0.79 ± 0.18* | 5.3 ± 1.0* |
| I(medium) | | | | | |
| polypeptide | 10 | 0.1 | 4.27 ± 0.85* | 1.18 ± 0.31* | 5.7 ± 1.1* |
| I(low) | | | | | |

*referring to p < 0.05,
**referring to p < 0.01.
CONCLUSION: polypeptide I has therapeutic effect on adjuvant-induced arthritis in rats

### Embodiment 4

### In Vivo Immunoprotective Effect of Polypeptide II on CIA Mouse Models

The test procedure is the same as embodiment 2, only polypeptide II is used instead of polypeptide I; polypeptide II is divided into low-dose (0.8 mg/kg), medium-dose (1.6 mg/kg) and high-dose (3.2 mg/kg) groups respectively and administered twice a day for 10 day in succession.

RESULTS: comparing the model mice with the normal mice. On day 0 of the experiment, the model mice are firstly hypodermically injected at the tail with an emulsion made by collagen and isovolumetric CFA (containing deactivated Mycobacterium tuberculosis); on day 21 of the experiment, the model mice are again hypodermically injected at the tail with an emulsion made by collagen and isovolumetric IFA; on day 27 of the experiment, swelling at paws appears on CIA mice and points for arthritis scoring start increasing; the highest degree of swelling on model mice appeared on day 45-60; in addition, the body weight of model mice stops increasing since day 35 and even slightly decreases later on. The in vivo immunoprotective effect of polypeptide II on CIA mouse models is shown in table 3. As is shown in the table, all doses of polypeptide II present immunoprotective effect on CIA mouse models:

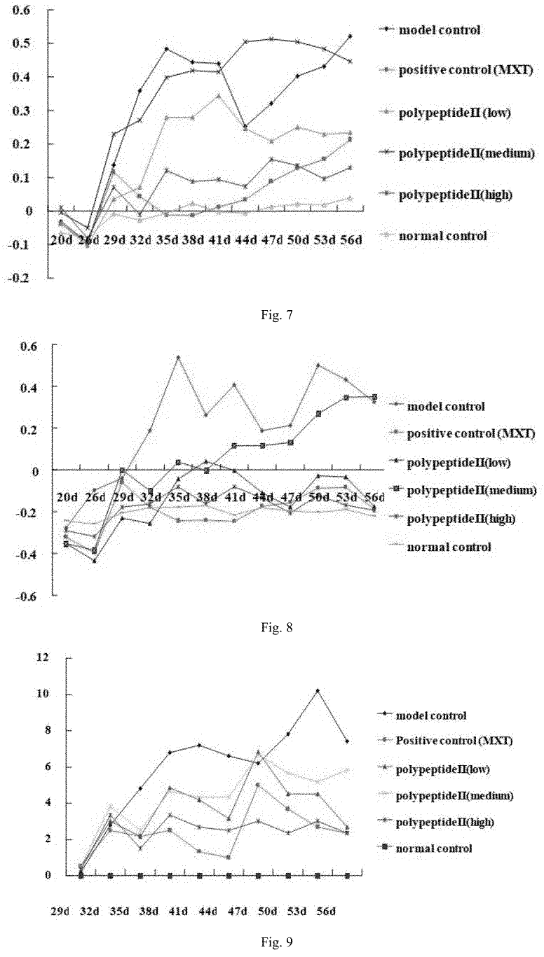

The effect of polypeptide II in improving paw swelling of CIA mouse models is demonstrated in FIG. 7. Insofar as the degree of paw swelling is concerned, both the positive control group and the polypeptide II low-dose group present extremely significant difference (p<0.01) in contrast with the model control group; the test result is statistically significant. The effect of polypeptide II in improving joint swelling of CIA mouse models is demonstrated in FIG. 8. Insofar as the degree of joint swelling is concerned, both the positive control group and the polypeptide II group (including high-dose and low-dose) present significant difference (p<0.05) in contrast with the model control group; the test result is statistically significant. The effect of polypeptide II in improving joint scoring of CIA mouse models is demonstrated in FIG. 9. The scoring points of polypeptide II group (both high and low doses) are greatly lower than that of the control group; there exists significant difference (P<0.05) between these two groups, and the test result is statistically significant.

TABLE 3
in vivo immunoprotective effect of polypeptide II on
CIA mouse models

| | num- | dose | | swelling | |
|---|---|---|---|---|---|
| | ber | (mg/ | swelling of | of joints | clinical |
| group | (n) | kg) | paws (mm) | (mm) | scoring |
| normal | 10 | — | 0.04 ± 0.07** | −0.22 ± | 0.00 ± 0.00** |
| control | | | | 0.12** | |
| model | 10 | — | 0.52 ± 0.46 | 0.32 ± | 7.40 ± 3.85 |
| control | | | | 0.60 | |
| positive | 10 | 1 | 0.21 ± 0.45 | −0.19 ± | 2.33 ± 2.73* |
| control | | | | 0.43* | |
| polypeptide | 10 | 3.2 | 0.23 ± 0.35 | −0.18 ± | 2.67 ± 2.34* |
| II(high) | | | | 0.49* | |
| polypeptide | 10 | 1.6 | 0.45 ± 0.41 | 0.35 ± | 5.83 ± 2.93 |
| II(medium) | | | | 0.80 | |
| polypeptide | 10 | 0.8 | 0.13 ± 0.28** | −0.19 ± | 2.33 ± 3.14* |
| II(low) | | | | 0.28* | |

*referring to p < 0.05,
**referring to p < 0.01.
CONCLUSION: polypeptide II has therapeutic effect on collagen-induced arthritis in mice

### Embodiment 5

### In Vivo Immunoprotective Effect of Polypeptide II on AIA Rat Models

The test procedure is the same as embodiment 3, only polypeptide II is used instead of polypeptide I; polypeptide II is divided into low-dose (0.4 mg/kg), medium-dose (0.8 mg/kg) and high-dose (1.6 mg/kg) groups respectively and administered three times a day for 10 day in succession. RESULTS: comparing the model rats with the normal rats. The primary arthritis appears at the left hind paw of model rats soon after injection of CFA containing deactivated Mycobacterium tuberculosis at the left hind paw, along with apparent swelling and ulceration; the secondary arthritis appears at the right high paw about 10 days later, with increasingly high scores; meanwhile, apparent angiogenesis occurs at rat ears, with obvious redness and swelling; swelling also appears at tail joints. The in vivo immunoprotective effect of polypeptide II on AIA rat models is shown in table 4. As is shown in the table, all doses of polypeptide II present immunoprotective effect on AIA rat models:

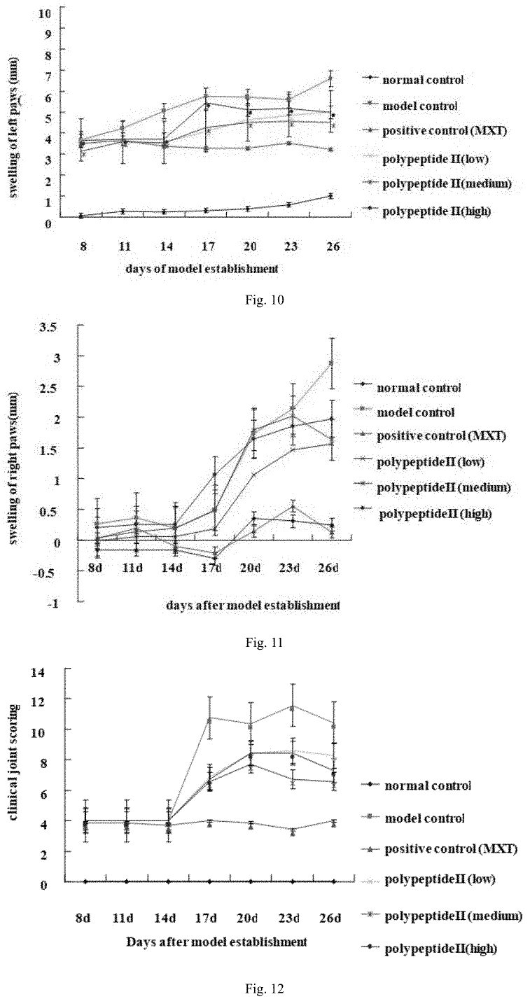

The effect of polypeptide II in improving the swelling degree of left paws induced by primary AIA is shown in FIG. 10. Insofar as the diameter of the left hind ankles is concerned, the positive control group presents extremely significant difference (p<0.01) in contrast with the model control group, whereas polypeptide II low-dose group presents significant difference (p<0.05) in contrast with the model control group; the test result is statistically significant. The effect of polypeptide II in improving the swelling degree of right paws induced by secondary AIA is shown in FIG. 11. Insofar as the diameter of the right hind ankles is concerned, both the positive control group and the polypeptide II lose-dose group present significant difference (p<0.05) in contrast with the model control group. The effect of polypeptide II in improving joint scoring of AIA rat models is demonstrated in FIG. 12. The scoring points of polypeptide II group (including medium and high doses) are lower than that of the control group; there exists significant difference (p<0.05) between these two groups, and the test result is statistically significant.

TABLE 4
in vivo immunoprotective effect of polypeptide II on AIA rat models

| | num- | dose | swelling of | swelling of | |
|---|---|---|---|---|---|
| | ber | (mg/ | left paw | right paw | clinical |
| group | (n) | kg) | (mm) | (mm) | scoring |
| normal | 10 | — | 1.00 ± 0.47** | 0.25 ± 0.28** | 0.00 ± 0.00** |
| control | | | | | |
| model | 10 | — | 6.59 ± 0.88 | 2.87 ± 1.27 | 10.43 ± 3.87 |
| control | | | | | |
| positive | 10 | 1 | 3.23 ± 1.20** | 0.13 ± 0.33** | 4.00 ± 0.00** |
| control | | | | | |
| polypeptide | 10 | 1.6 | 4.98 ± 1.97 | 1.97 ± 1.56 | 7.29 ± 3.40* |
| II(high) | | | | | |
| polypeptide | 10 | 0.8 | 4.50 ± 1.95* | 1.63 ± 1.57 | 6.57 ± 4.24* |
| II(medium) | | | | | |
| polypeptide | 10 | 0.4 | 5.03 ± 1.99 | 1.56 ± 1.66* | 8.29 ± 4.86 |
| II(low) | | | | | |

*referring to p < 0.05,
**referring to p < 0.01.
CONCLUSION: polypeptide II has therapeutic effect on adjuvant-induced arthritis, and its effect is higher than that of polypeptide I.

### Embodiment 6

### In Vivo Immunoprotective Effect of Polypeptide III on CIA Mouse Models

The test procedure is the same as embodiment 2, only polypeptide III is used instead of polypeptide I; polypeptide III is divided into low-dose (10 mg/kg), medium-dose (20 mg/kg) and high-dose (40 mg/kg) groups respectively and administered once the other day, 5 times in total.

RESULTS: comparing the model mice with the normal mice. On day 0 of the experiment, the model mice are firstly hypodermically injected at the tail with an emulsion made by collagen and isovolumetric CFA (containing deactivated Mycobacterium tuberculosis); on day 21 of the experiment, the model mice are again hypodermically injected at the tail with an emulsion made by collagen and isovolumetric IFA; on day 27 of the experiment, swelling at paws appears on CIA mice and points for arthritis scoring start increasing; the highest degree of swelling on model mice appeared on day 45-60; in addition, the body weight of model mice stops increasing since day 35 and even slightly decreases later on. The in vivo immunoprotective effect of polypeptide III on CIA mouse models is shown in table 5. As is shown in the table, all doses of polypeptide III present immunoprotective effect on CIA mouse models:

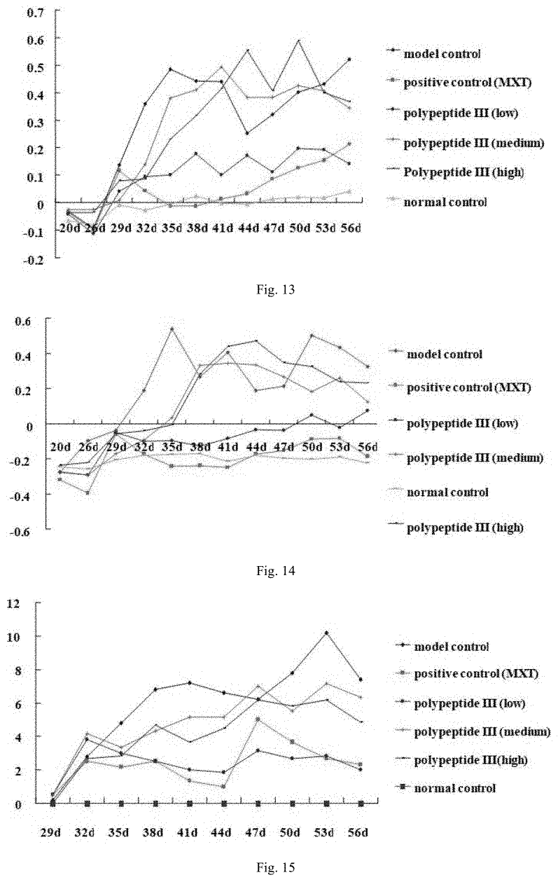

The effect of polypeptide III in improving paw swelling of CIA mouse models is demonstrated in FIG. 13. Insofar as the diameter of ankles is concerned, both the positive control group and the polypeptide III high-dose group present significant difference (p<0.05) in contrast with the model control group; the test result is statistically significant. The effect of polypeptide III in improving joint swelling of CIA mouse models is demonstrated in FIG. 14. Insofar as the degree of joint swelling is concerned, both the positive control group and the polypeptide III high-dose group present significant difference (p<0.05) in contrast with the model control group; the test result is statistically significant. The effect of polypeptide III in improving joint scoring of CIA mouse models is demonstrated in FIG. 15. The scoring points of polypeptide III high-dose group are greatly lower than that of the control group; there exists significant difference (P<0.05) between these two groups, and the test result is statistically significant.

TABLE 5
in vivo immunoprotective effect of polypeptide III on
CIA mouse models

| | num- | dose | | swelling | |
|---|---|---|---|---|---|
| | ber | (mg/ | swelling of | of joints | clinical |
| group | (n) | kg) | paws (mm) | (mm) | scoring |
| normal | 10 | — | 0.04 ± 0.07** | −0.22 ± | 0.00 ± 0.00** |
| control | | | | 0.12** | |
| model | 10 | — | 0.52 ± 0.46 | 0.32 ± | 7.40 ± 3.85 |
| control | | | | 0.60 | |
| positive | 10 | 1 | 0.21 ± 0.45* | −0.19 ± | 2.33 ± 2.73* |
| control | | | | 0.43* | |
| polypeptide | 10 | 40 | 0.14 ± 0.42* | 0.07 ± | 2.00 ± 2.37* |
| III(high) | | | | 0.08* | |
| polypeptide | 10 | 20 | 0.34 ± 0.42 | 0.12 ± | 6.33 ± 4.23 |
| III(medium) | | | | 0.55 | |
| polypeptide | 10 | 10 | 0.37 ± 0.35 | 0.23 ± | 4.83 ± 2.99 |
| III(low) | | | | 0.43 | |

*referring to p < 0.05,
**referring to p < 0.01.
CONCLUSION: polypeptide III has therapeutic effect on collagen-induced arthritis in mice

### Embodiment 7

### In Vivo Immunoprotective Effect of Polypeptide III on AIA Rat Models

The test procedure is the same as embodiment 3, only polypeptide III is used instead of polypeptide I; polypeptide III is divided into low-dose (10 mg/kg), medium-dose (20 mg/kg) and high-dose (40 mg/kg) groups respectively and administered once the other day, 5 times in total.

RESULTS: comparing the model rats with the normal rats. The primary arthritis appears at the left hind paw of model rats soon after injection of CFA containing deactivated Mycobacterium tuberculosis at the left hind paw, along with apparent swelling and ulceration; the secondary arthritis appears at the right high paw about 10 days later, with increasingly high scores; meanwhile, apparent angiogenesis occurs at rat ears, with obvious redness and swelling; swelling also appears at tail joints. The in vivo immunoprotective effect of polypeptide III on AIA rat models is shown in table 6. As is shown in the table, all doses of polypeptide III present immunoprotective effect on AIA rat models:

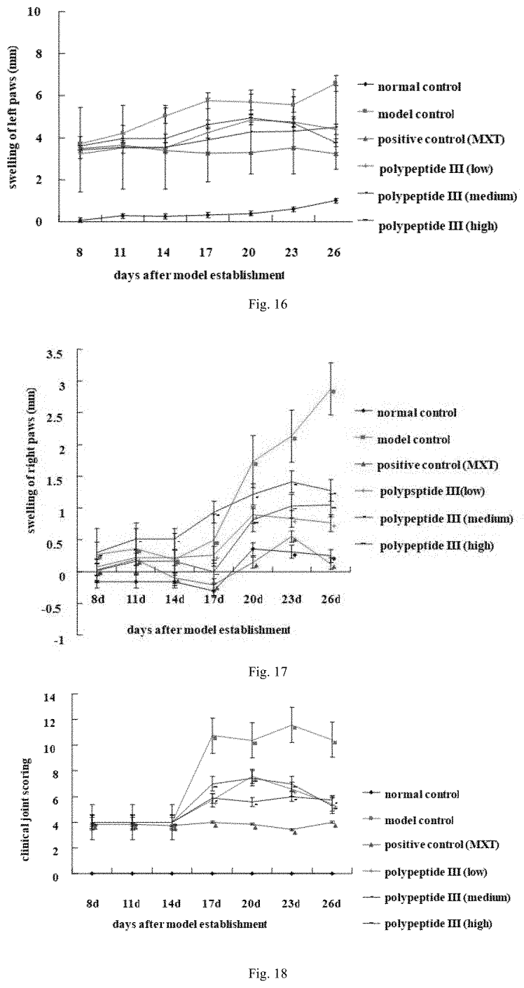

The effect of polypeptide III in improving the swelling degree of left paws induced by primary AIA is shown in FIG. 16. Insofar as the diameter of the left hind ankles is concerned, both the positive control group and polypeptide III low-dose group present extremely significant difference (p<0.01) in contrast with the model control group, whereas both polypeptide III medium-dose group and polypeptide III high-dose group present significant difference (p<0.05) in contrast with the model control group; the test result is statistically significant. The effect of polypeptide III in improving the swelling degree of right paws induced by secondary AIA is shown in FIG. 17. Insofar as the diameter of the right hind ankles is concerned, both the positive control group and polypeptide III low-dose group present extremely significant difference (p<0.01) in contrast with the model control group, whereas both the polypeptide III medium-dose group and the polypeptide III high-dose group present significant difference (p<0.05) in contrast with the model control group; the test result is statistically significant. The effect of polypeptide III in improving joint scoring of AIA rat models is demonstrated in FIG. 18. The scoring points of polypeptide III group (including low, medium and high doses) are lower than that of the control group; there exists extremely significant difference (p<0.01) between these two groups, and the test result is statistically significant.

TABLE 6
in vivo immunoprotective effect of polypeptide III on AIA rat models

| | num- | dose | swelling of | swelling of | |
|---|---|---|---|---|---|
| | ber | (mg/ | left paw | right paw | clinical |
| group | (n) | kg) | (mm) | (mm) | scoring |
| normal | 10 | — | 1.00 ± 0.47** | 0.25 ± 0.28** | 0.00 ± 0.00** |
| control | | | | | |
| model | 10 | — | 6.59 ± 0.88 | 2.87 ± 1.27 | 10.43 ± 3.87 |
| control | | | | | |
| positive | 10 | 1 | 3.23 ± 1.20** | 0.13 ± 0.33** | 4.00 ± 0.00** |
| control | | | | | |
| polypeptide | 10 | 40 | 4.40 ± 0.91* | 1.05 ± 1.31* | 5.29 ± 3.90** |
| III(high) | | | | | |
| polypeptide | 10 | 20 | 4.48 ± 1.72* | 1.28 ± 1.27* | 5.71 ± 2.36** |
| III(medium) | | | | | |
| polypeptide | 10 | 10 | 4.40 ± 0.91** | 0.76 ± 1.21** | 5.43 ± 2.15** |
| III(low) | | | | | |

*referring to p < 0.05,
**referring to p < 0.01.
CONCLUSION: polypeptide III has therapeutic effect on adjuvant-induced arthritis, and its effect is higher than that of polypeptide. I.

### Embodiment 8

### Effect on IL-1β Production by Mouse Peritoneal Macrophages

IL-1β is not only an immunoregulatory factor, but also an inflammatory mediator, which plays an important role in the occurrence and development of rheumatoid diseases. The purpose of this test is to detect the impact of mPEG-SC₂₀ₖ-HM-3 on the expression of IL-1β in mouse peritoneal macrophages, to understand the impact of polypeptide mPEG-SC₂₀ₖ-HM-3 on the antigen presentation ability and inflammatory mediator release ability of peritoneal macrophages, and to clarify whether polypeptide mPEG-SC₂₀ₖ-HM-3 can play an anti-inflammatory and inflammatory-relieving role by inhibiting the expression of IL-1β.

### Test Grouping

In the test, mice are divided into 11 groups, namely a blank control group, a lipopolysaccharide (LPS) model group, a dexamethasone group, and mPEG-SC₂₀ₖ-HM-3 groups at different doses (0.56, 1.12, 2.25, 4.50, 9.00 μmol/L).

### III. Test Drug

1. Name: Dexamethasone Dex

Batch No.: 12100511

Manufacturer: Jiangsu Lianshui Pharmaceutical Co., Ltd.

Preparation method: The required concentration is obtained using a blank medium before administration, and the drug is store at −20° C. for later use.

2. Name: mPEG-SC₂₀ₖ-HM-3

Batch No.: 130228

Manufacturer: Synthesized by our laboratory

Preparation method: The required concentration is obtained using a blank medium before administration, and the drug is store at −20° C. for later use.

### IV. Others

Name: Lipopolysaccharide (LPS)

Batch No.: 033M4054V

Manufacturer: SIGMA

Preparation method: The required concentration is obtained using a blank medium before the test, and the drug is store at 4° C. for later use.

### V. Animals

Strain, source: C57BL/6 mice, age: 6-8 weeks; weight: 20-25 g; gender: male. Provided by Comparative Medicine Center of Yangzhou University, laboratory animal production license: SCXK (Su) 2012-0004; certificate number: 0025474; invoice number: 01286246; Number of the using of Laboratory Animal. SYXK (Su) 2011-0036.

### VI. Test Method

(1) IL-1 production: mice are intraperitoneally injected with 1 mL of broth medium (containing 6% of starch). After three days, mouse peritoneal macrophages are taken aseptically and washed twice with RPMI 1640 medium, and the cell concentration is adjusted to 2×10⁶ cells/mL. The cells are injected into a 24-well culture plate, 1 mL per well, and the cells are incubated in a cell incubator for 3 h, and vibrated once every 30 min to make the cells fully adhere. Then the cells are washed twice with culture solution to remove non-adherent cells, are cultured continuously for 48 h after administration, and are centrifuged at 1000 r for 15 min. The supernatant is collected as a sample with IL-1β to be tested. (2) Determination of IL-1 content: various solutions are prepared; tested samples and standard substances with different concentrations (100 μL/well) are added into the corresponding wells, reaction wells are sealed with plate-sealing adhesive tape, and incubation is performed at 37° C. for 90 min; the plate is washed four times; biotinylated antibody working solution (100 μL/well) is added, the reaction wells are sealed with plate-sealing adhesive tape, and incubation is performed at 37° C. for 60 min; the plate is washed four times; enzyme conjugate working solution (100 μL/well) is added, the reaction wells are sealed with plate-sealing adhesive tape, and incubation is performed at 37° C. for 30 min; the plate is washed four times; chromogenic reagent (100 μL/well) is added, and incubation is performed at 37° C. for 10-20 min in the absence of light; stop solution (100 μL/well) is added, and the OD 450 nm absorbance is measured immediately after uniform mixing (within 5 min).

### VII. Test Results

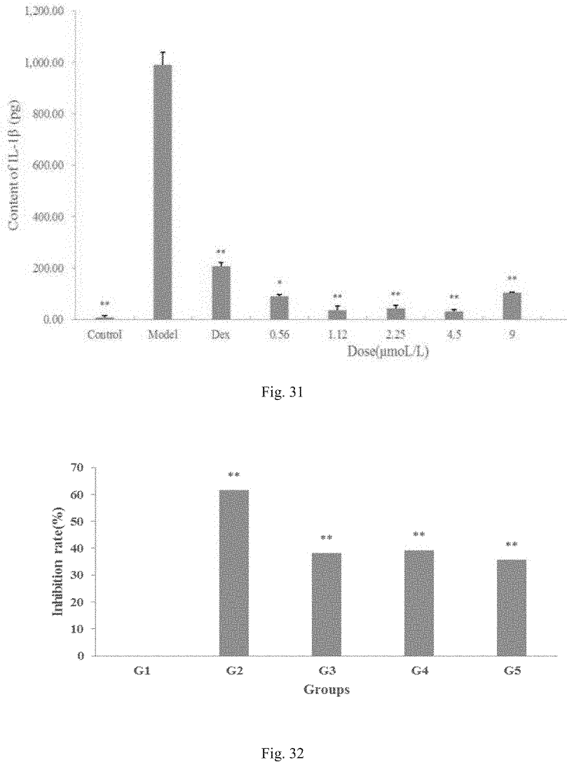

Test results (see Table 7 and FIG. 31): compared with the model group, each of mPEG-SC₂₀ₖ-HM-3 groups at different doses can inhibit the production of IL-1β by mouse peritoneal macrophages, presenting good inhibition rates at a dose range of 0.56-9 μmol/L, which are 90.93%, 96.45%, 95.58%, 96.76% and 89.41%. The inhibition rate at a dose of 4.5 μmol/L is the highest and is 96.76%. They are all higher than the inhibition rate, 78.97%, of the positive drug group Dex, and all have significant or extremely significant differences (**p<0.01). mPEG-SC₂₀ₖ-HM-3 can inhibit the production of IL-1β by mouse peritoneal macrophages, showing a good inverse U shape within the dose range of 0.56-9 μmol/L, and has a good anti-inflammatory effect.

TABLE 7
The impact of mPEG-SC₂₀ₖ-HM-3 on the secretion of IL-1β from macrophage

| | | | LPS | | |
|---|---|---|---|---|---|
| | | Dosage | Concentration | IL-1β content | |
| Groups (n = 5) | | (μmoL/L) | (μg/mL) | (pg/mL) | Inhibition rate (%) |
| Control | | 0 | 0 | 7.88 ± 6.95** | — |
| Model | | 0 | 5 | 989.82 ± 49.45 | — |
| Dex | | 50 | 5 | 208.19 ± 13.78** | 78.97 |
| | | 0.56 | 5 | 89.77 ± 7.22* | 90.93 |
| mPEG-SC₂₀ₖ-HM-3 | 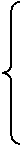 | 1.12 2.25 4.50 9.00 | 5 5 5 5 | 35.17 ± 17.71** 43.78 ± 10.16** 32.04 ± 6.52** 104.83 ± 2.44** | 96.45 95.58 96.76 89.41 |

*p < 0.05,
**p < 0.01 vs model group.

### Embodiment 9

### Effect on Subacute Inflammation of Cotton Ball Granuloma in Rats

### I. Test Objectives

To explore whether subcutaneous injection of high, medium and low doses of mPEG-SC₂₀ₖ-HM-3 can inhibit cotton ball granuloma in rats.

### II. Test Grouping

The rats are randomly divided into a model control group, a positive control (dexamethasone) group, and mPEG-SC₂₀ₖ-HM-3 groups using subcutaneous injection at a high dose, a medium dose and a low dose, a total of 5 groups, and the drugs are administered to each group of animals by subcutaneous injection, for 7 days in succession. Specific dose regimen is shown in Table 8.

TABLE 8
Dose regimen

| Groups | Drug arrangement | days, dose | Administration |
|---|---|---|---|
| G1 | Artificial sera | d 1, 2, 3, 4, 5, 6, 7; | sc. |
| | | 7 days, 1 mL/100 g | |
| G2 | Dexamethasone | d 1, 2, 3, 4, 5, 6, 7; | sc. |
| | | 7 days, 5 mg/kg, | |
| | | 1 mL/100 g | |
| G3 | mPEG-SC₂₀ₖ-HM-3 | d 1, 3, 5, 7; 7 days, | sc. |
| | high-dose | 20 mg/kg, 1 mL/100 g | |
| G4 | mPEG-SC₂₀ₖ-HM-3 | d 1, 3, 5, 7; 7 days, | sc. |
| | medium-dose | 10 mg/kg, 1 mL/100 g | |
| G5 | mPEG-SC₂₀ₖ-HM-3 | d 1, 3, 5, 7; 7 days, | sc. |
| | low-dose | 5 mg/kg, 1 mL/100 g | |

### III. Test Drug

1. Name: Normal saline

Batch No.: 12040313301

Manufacturer: Cisen Pharmaceutical Co., Ltd.

2. Name: Dexamethasone

Batch No.: 1106013

Manufacturer: Jiangsu Lianshui Pharmaceutical Co., Ltd.

Preparation method: The required concentration is obtained using normal saline before administration, and the drug is store at −20° C. for later use.

3. Name: mPEG-SC₂₀ₖ-HM-3

Batch No.: 120625

Manufacturer: Synthesized by our laboratory

Preparation method: The required concentration is obtained using normal saline before administration, and the drug is store at −20° C. for later use.

### IV. Animals

Strain, source: SPF SD rats, age: 6-7 weeks; weight: 180-200 g; gender: half male and half female, n=8. They are purchased from Shanghai Xipuer-Bike Experimental Animal Co., Ltd. Production License: SCXK (Hu) 2008-0016 Certificate number: 2008001624534; invoice number: 02998322; Number of the using of Laboratory Animal: SYXK (Su) 2011-0036.

### V. Test Method

Rats in each group are anesthetized with pentobarbital sodium (40 mg/kg) by intraperitoneal injection, and the abdominal coat is cut off. The skin in the middle of the lower abdomen is cut open under aseptic condition, and the incision is about 1 cm long. The subcutaneous tissue is expanded by vascular forceps, and 30 mg of sterile dry cotton balls (weighed accurately by an electronic balance before the test, and undergoing conventional autoclaving and drying for later use) are implanted hypodermically into the groin on one side. The incision is sutured, and an appropriate amount of amoxicillin is sprinkled on the incision to prevent infection.

The drug is administered on the same day after surgery, and is administered for 7 days in succession. On day 8, the rats are killed by cervical dislocation, the cotton balls are removed surgically, the fat tissue is removed, and the rats are dried at 60° C. The mass of granuloma is expressed in mg (granulomatoma)/100 g (body weight). The granuloma mass difference between each administration group and the model control group is compared, and the inhibition rate is calculated. Data statistical analysis is performed on significant differences between the groups.

Inhibition rate (%)=(average granuloma mass of model control group−average granuloma mass of administration group)/average granuloma mass of model control group×100%.

### VI. Test Results

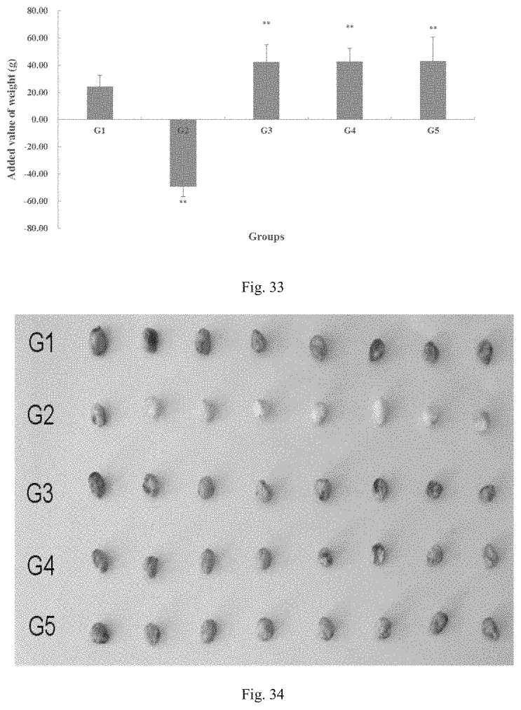

Test results are shown in Tab 9, Tab 10 and FIG. 32-34. Compared with the model control group, mPEG-SC₂₀ₖ-HM-3 can inhibit cotton ball granuloma in rats at high, medium and low doses, with an extremely significant difference (**p<0.01), especially the medium dose (10 mg/kg) has the best effect, and its inhibition rate is 39.33%. The inhibitory rates at a high dose (20 mg/kg) and a low dose (5 mg/kg) are 38.23% and 35.67%, respectively, but there is no significant difference among the three groups. Test results show that each mPEG-SC₂₀ₖ-HM-3 group can inhibit cotton ball granuloma in rats, and shows its good ability of resisting proliferation of connective tissues. Compared with the model control group, the weight of mice in the positive drug Dex group decreases significantly, and the change is (−49.50±7.39) g (**p<0.01). Compared with that of the model control group, the weight of mice in each of the mPEG-SC₂₀ₖ-HM-3 groups at a high dose (20 mg/kg), medium dose (10 mg/kg) and low dose (5 mg/kg) increases significantly, and the weight changes are (42.25±12.78) g (*p<0.05), (42.63±9.59) g (**p<0.01), and (42.88±17.77) g (*p<0.05) respectively. Compared with that of the positive drug Dex group, the weight of each of the mPEG-SC₂₀ₖ-HM-3 groups increases significantly, indicating that mPEG-SC₂₀ₖ-HM-3 is safer and enhances the body's inflammatory resistance. mPEG-SC₂₀ₖ-HM-3 groups at different doses can extremely significantly inhibit subacute inflammation of cotton ball granuloma in rats.

TABLE 9
The impact of mPEG-SC₂₀ₖ-HM-3 on the cotton ball granuloma of rats (mean ± SD, n = 8)

| | | | | | cotton ball granuloma | |
|---|---|---|---|---|---|---|
| | | | | | mg(cotton ball | |
| | | | Dose | Weight | granuloma)/100 | |
| Groups | | N | (mg/kg) | added(g) | g(weight) | Inhibition rate(%) |
| Model | | 8 | — | 24.25 ± 8.56 | 0.29 ± 0.05 | — |
| Dexamethasone | | 8 | 5 | −49.50 ± 7.39** | 0.11 ± 0.04** | 61.59** |
| mPEG-SC₂₀ₖ-HM-3 | 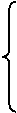 | 8 8 8 | 20 10 5 | 42.25 ± 12.78* 42.63 ± 9.59** 42.88 ± 17.77* | 0.18 ± 0.03** 0.18 ± 0.02** 0.19 ± 0.03** | 38.23** 39.33** 35.67** |

vs model group,
*p < 0.05,
**p < 0.01.

TABLE 10
The impact of mPEG-SC₂₀ₖ-HM-3 on the added value of rats weight.

| | Added value of rats weight (g) | | | | | | | | |
|---|---|---|---|---|---|---|---|---|---|
| Groups | 1 | 2 | 3 | 4 | 5 | 6 | 7 | 8 | Mean ± SD |
| G1 | 16 | 32 | 38 | 27 | 28 | 14 | 23 | 16 | 24.25 ± 8.56 |
| G2 | −49 | −55 | −37 | −57 | −43 | −49 | −59 | −47 | −49.50 ± 7.39** |
| G3 | 52 | 52 | 44 | 26 | 19 | 53 | 47 | 45 | 42.25 ± 12.78** |
| G4 | 27 | 45 | 59 | 43 | 47 | 39 | 34 | 47 | 42.63 ± 9.59** |
| G5 | 57 | 40 | 10 | 43 | 67 | 55 | 29 | 42 | 42.88 ± 17.77** |

*p < 0.05,
**p < 0.01 vs model group.

### Embodiment 10

### Effect on Acute Inflammation of Toe Swelling in Carrageenan-Induced Transgenic TNF-luc Mice

### I. Test Objectives

To study the inhibitory and alleviating effect of mPEG-SC₂₀ₖ-HM-3 on acute inflammation of toe swelling in Balb/C transgenic TNF-luc mice induced by carrageenan.

### II. Drug Tested

Name: mPEG-SC₂₀ₖ-HM-3

Provider: Synthesized by our laboratory, batch No.: 140408

### III. Control Drugs

1. Name: Adalimumab; manufacturer: AbbVie; specification: 40 mg/0.8 mL, batch No.: 28388LX01

2. Name: 0.9% sodium chloride injection; manufacturer: Cisen Pharmaceutical Co., Ltd.; specification:

500 mL:4.5 g, batch No.: 1303207021

### IV. Animal Grouping and Dose Setting

TABLE 11
Dosing regimen.

| | | | | | | Administration | | |
|---|---|---|---|---|---|---|---|---|
| | | | Induced | | Administrated | time and drug | Fluorescein | |
| Groups | N | Induction methods | dose | Treatment | dose | delivery | delivery | Detection |
| Normal | 2 | / | / | Saline | 100 μL/20 g | −1 h sc. | mg/20 g | Small animal in vivo |
| Model | 2 | λ-Carrageenan; | 1%; | Saline | 100 μL/20 g | −1 h sc. | i.p. | imaging instrument |
| | | Subcutaneous injection in | 50 μL | | | | | 430 nm/535 nm |
| Positive | 2 | foot palm | | Adalimumab | 8 mg/kg | −1 h sc. | | |
| Test | 2 | | | mPEG-SC₂₀ₖ-HM-3 | 20 mg/kg | −1 h sc. | | |

### V. Drug Preparation Method

1.1% carrageenan: 0.02142 g (0.02 g is required) of carrageenan is weighed and placed into a 4 mL EP tube, 2 mL of normal saline is added, the drug is uniformly dissolved in hot water at 80° C. and stands at 4° C. for later use.

2. Positive drug group: 16 μL of Adalimumab original medicine solution is added to 984 μL of normal saline injection to reach a concentration of 0.8 mg/mL, and the administration volume is 0.2 mL/20 g of body weight.

3. Test administration group: 0.01733 g (0.016 g is required) of mPEG-SC₂₀ₖ-HM-3 powder is weighed and put into 8 mL of normal saline to reach a concentration of 2 mg/mL, and the administration volume is 0.2 mL/20 g of body weight.

(The sterile normal saline is used for preparation before each administration.)

### VI. Animals

Source, strain: SPF BALB/c transgenic TNF-luc mice with genetic background, provided by Model Animal Research Center of Nanjing University (laboratory animal production license No.: SCXK (Su) 2015-0001; laboratory animal quality certificate: 201500002), Number of the using of Laboratory Animal. SYXK (Jun) 2012-0049.

Age: 6-7 weeks at the time of purchase and 7-8 weeks at the beginning of administration;

weight: (18±22) g at the time of purchase and (20±24) g at the beginning of administration;

gender: half male and half female;

number of animals in each group: half male and half female (n=2).

### VII. Inflammatic Agent

Name: λ-Carrageenan; manufacturer: Sigma-Aldrich Inc.; specification: 5 g; batch No.: 0001408463

### VIII. Luminescence Reagent

Name: VivoGlo™ Luciferin, In Vivo Grade; manufacturer: Promega Corporation;

specification: 50 mg; batch No.:124359179

### IX. Test Instruments

Electronic balance, BT25S Sartorius Scientific Instrument (Beijing) Co., Ltd.

ChemBase CBS-CJ-1FD super clean bench, Suzhou AIRTECH Air Technology Co., Ltd.

1 mL of Sterile disposable syringe (0.5×20 RWLB), Jiangsu Changcheng Medical Equipment Co., Ltd.

0-150 type vernier caliper, Taiwan Southwest Jiahua Precision Measuring Tool Co., Ltd.

Small animal in vivo imager, Carestream Health, Inc.

### X. Test Method

1. A model of inflammation of acute toe swelling in carrageenan-induced transgenic TNF-luc mice is established.

Before modeling, 8 animals are randomly divided into 4 groups (n=2), a normal group, a model group, a positive drug group and a test treatment group. One hour before modeling, the normal group and the model group are hypodermically injected with normal saline (0.2 mL/20 g of body weight) respectively; the positive drug group is hypodermically injected with Adalimumab (8 mg/kg of body weight), 15 d/time, once; and the test treatment group is hypodermically injected with mPEG-SC₂₀ₖ-HM-3 (20 mg/kg of body weight), 2 d/time, twice. The initial ankle width, foot palm width and toe thickness of the left foot are measured as initial values.

Modeling starts at hour 0, 50 μL of 1% carrageenan is hypodermically injected into the bottom of left toe of transgenic TNF-luc mice, and the ankle swelling degree, foot palm swelling degree and toe swelling degree of each group of mice are measured with a vernier caliper at 9 time points, hour 1, hour 3, hour 5, hour 7, hour 9, hour 12, hour 24, hour 48, hour 72 and hour 96 respectively. Method for calculating toe swelling degree:

Swelling degree of left ankle (cm)=swelling thickness of left ankle during measurement−thickness of left ankle during initial measurement.

Swelling degree of left foot palm (cm)=swelling thickness of left foot palm during measurement−thickness of left foot palm during initial measurement.

Swelling degree of left toe (cm)=swelling thickness of left toe during measurement−thickness of left toe during initial measurement.

2. In Vivo Imaging Evaluation of Mice

At 8 time points, hour 0, hour 2, hour 4, hour 8, hour 12, hour 24, hour 48 and hour 72 after modeling, fluorescein is injected intraperitoneally into each group of mice. After the mice are lightly anesthetized with ether, the mice are fixed within 5-20 min and put into an in vivo imager, the imager is covered with an instrument lid, on-fluro and om-photo names in the desktop imaging interface of the computer are clicked, and the expose button is clicked to complete the fluorescence and white light photos of mice in vivo imaging. The specific imaging principle, operation steps, image processing and output are the same as those in the previous embodiment.

### XI. Data Statistics

Test results are expressed by x±s, and the t test between each group and the control group is performed by using SPSS11.0 software. * means p<0.05, and ** means p<0.01.

In vivo imaging results of mice are processed with Carestream Molecular Imaging Software 5.3, and the luminous area is used as the determination of results. The fluorescence areas of the groups after optimization are compared.

### XII. Results and Discussions

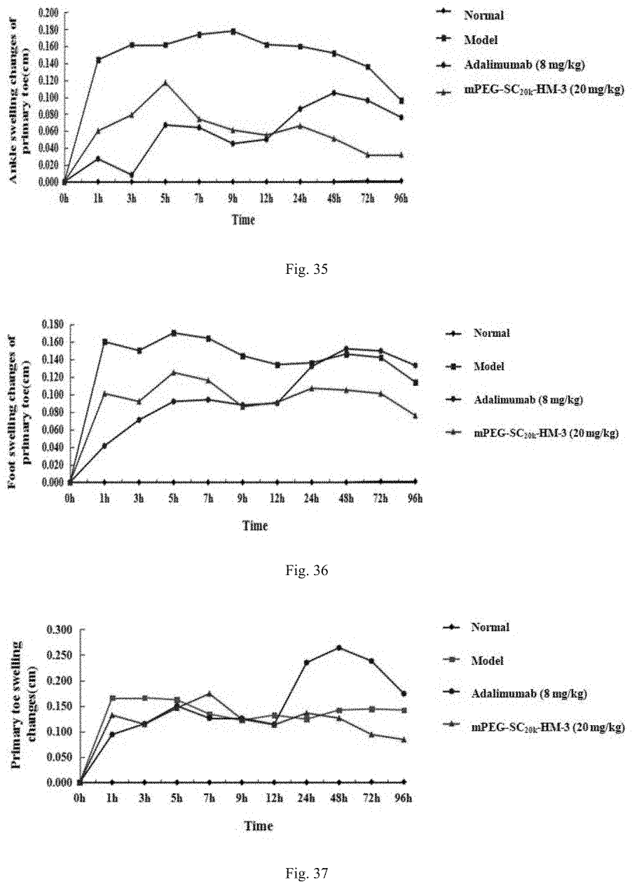

Results of mouse ankle swelling (in Tab 12, FIG. 35) show that the mPEG-SC₂₀ₖ-HM-3 group (20 mg/kg) can effectively inhibit the swelling of left ankle of the TNF-luc mice model at time points, hour 7, hour 9, hour 12, hour 24, hour 48, hour 72 and hour 96. Compared with that of the positive drug Adalimumab (8 mg/kg) group, the ankle swelling degrees of the mPEG-SC₂₀ₖ-HM-3 group (20 mg/kg) at hour 48, hour 72 and hour 96 are (0.051±0.044), (0.032±0.074), (0.032±0.042), respectively, with a significant difference (*p<0.05); and compared with that of the model group, they are extremely significantly different (**p<0.01).

Results of mouse foot palm swelling (in Tab 13, FIG. 36) show that the mPEG-SC₂₀ₖ-HM-3 group (20 mg/kg) can effectively inhibit the swelling of left foot palm of the TNF-luc mice model at time points, hour 3 and hour 96. Compared with that of the positive drug Adalimumab (8 mg/kg) group, the foot palm swelling degrees of the mPEG-SC₂₀ₖ-HM-3 (20 mg/kg) at hour 3 and hour 96 are (0.092±0.054) and (0.076±0.006), respectively, with an extremely significant difference (**p<0.01); and compared with that of the model group, they are extremely significantly different (**p<0.01).

Results of mouse toe swelling (in Tab 14, FIG. 37) show that the mPEG-SC₂₀ₖ-HM-3 group (20 mg/kg) can effectively inhibit the swelling of left toe of the TNF-luc mice model at time points, hour 72 and hour 96. Compared with that of the positive drug Adalimumab (8 mg/kg) group, the foot palm swelling degrees of the mPEG-SC₂₀ₖ-HM-3 group (20 mg/kg) at hour 72 and hour 96 are (0.094±0.034) and (0.084±0.040), respectively, with an extremely significant difference (**p<0.01); and compared with that of the model group, they are extremely significantly different (**p<0.01).

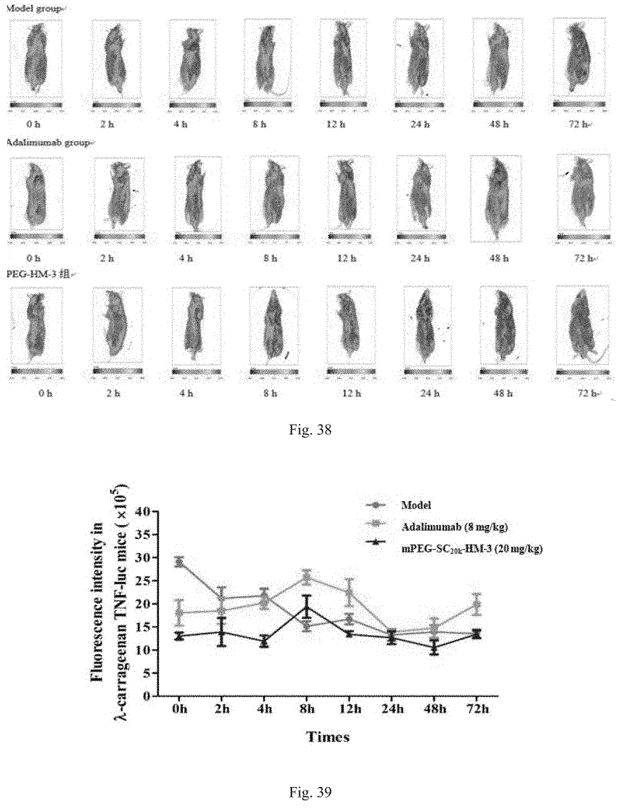

In vivo imaging results (in Tab 15, FIG. 38, FIG. 39) show that, at hour 8, hour 12, hour 24, hour 48 and hour 72, the in vivo fluorescence areas (10⁵) in the model group are (15.15±1.83), (16.68±1.96), (13.25±2.22), (13.91±2.10) and (13.52±1.27), respectively. The in vivo fluorescence areas (10⁵) of mice in the mPEG-SC₂₀ₖ-HM-3 group (20 mg/kg) decrease to (19.37±3.41), (18.65±9.09), (12.65±2.39), (14.37±6.24) and (13.40±1.17) respectively. In the Adalimumab group (8 mg/kg), they are (25.70±2.67), (22.40±5.06), (13.89±8.53), (14.76±3.58) and (19.87±3.92), respectively, and there is no obvious decrease. Compared with that of the model group, the in vivo fluorescence area in the mPEG-SC₂₀ₖ-HM-3 group decreases significantly, with an extremely significant difference (***p<0.001). Compared with that of the positive drug Adalimumab group, the in vivo fluorescence area in the mPEG-SC₂₀ₖ-HM-3 group is extremely significantly different (***p<0.001). It indicates that the mPEG-SC₂₀ₖ-HM-3 group has a better inhibitory effect than the positive drug Adalimumab group in inhibiting the expression of TNF-α in mice.

### XIII. Conclusions

The mPEG-SC₂₀ₖ-HM-3 group can effectively inhibit acute ankle swelling, foot palm swelling and toe swelling in mice induced by carrageenan, which is significantly different from that of the positive drug Adalimumab group (*p<0.05). The mPEG-SC₂₀ₖ-HM-3 group has a significant inhibitory effect, which is extremely significantly different from that of the model group (**p<0.01).

In vivo imaging of mice indicates that the mPEG-SC₂₀ₖ-HM-3 group (20 mg/kg) inhibits the expression of TNF in mice. Compared with that of the positive group, the inhibitory effect of the mPEG-SC₂₀ₖ-HM-3 group (20 mg/kg) is better than that of the positive drug Adalimumab group (8 mg/kg); and compared with that of the model group, the inhibitory effect of the mPEG-SC₂₀ₖ-HM-3 group (20 mg/kg) has a significant difference.

### XIV. The Test Results are Summarized as Follows

TABLE 12
The impact of mPEG-SC₂₀ₖ-HM-3 on acute λ-carrageenan inflammation of left ankle swelling changes in TNF-luc mouse.
(mean ± SD, N = 2)

| | | | Times | | | | | |
|---|---|---|---|---|---|---|---|---|
| Groups | N | Dosage | 1 h | 3 h | 5 h | 7 h | 9 h | 12 h |
| Normal | 2 | / | 0.000 ± 0.000** | 0.000 ± 0.000** | 0.000 ± 0.000** | 0.000 ± 0.000** | 0.000 ± 0.000** | 0.000 ± 0.000** |
| Model | 2 | / | 0.144 ± 0.012 | 0.162 ± 0.024 | 0.162 ± 0.018 | 0.174 ± 0.033 | 0.178 ± 0.040 | 0.162 ± 0.032 |
| Adalimumab | 2 | 8 | 0.027 ± 0.018** | 0.008 ± 0.048** | 0.067 ± 0.010** | 0.064 ± 0.006** | 0.045 ± 0.004** | 0.050 ± 0.003** |
| mPEG-SC₂₀ₖ-HM-3 | 2 | 20 | 0.060 ± 0.045** | 0.079 ± 0.052** | 0.117 ± 0.033* | 0.074 ± 0.014** | 0.061 ± 0.013** | 0.055 ± 0.018** |

| | | | Times | | | |
|---|---|---|---|---|---|---|
| Groups | N | Dosage | 24 h | 48 h | 72 h | 96 h |
| Normal | 2 | / | 0.000 ± 0.000** | 0.000 ± 0.000** | 0.001 ± 0.002** | 0.001 ± 0.001** |
| Model | 2 | / | 0.160 ± 0.013 | 0.152 ± 0.014 | 0.136 ± 0.02 | 0.096 ± 0.003 |
| Adalimumab | 2 | 8 | 0.086 ± 0.031** | 0.105 ± 0.075* | 0.096 ± 0.051* | 0.076 ± 0.034* |
| mPEG-SC₂₀ₖ-HM-3 | 2 | 20 | 0.066 ± 0.003** | 0.051 ± 0.044** | 0.032 ± 0.074** | 0.032 ± 0.042** |

vs. model group, *p < 0.05,
**p < 0.01.

TABLE 13
The impact of mPEG-SC₂₀ₖ-HM-3 on acute λ-carrageenan inflammation of left foot swelling changes in TNF-luc mouse.
(mean ± SD, N = 2)

| | | Dosage | Times | | | | | |
|---|---|---|---|---|---|---|---|---|
| Groups | N | (mg/kg) | 1 h | 3 h | 5 h | 7 h | 9 h | 12 h |
| Normal | 2 | / | 0.000 ± 0.000** | 0.000 ± 0.000** | 0.000 ± 0.000** | 0.000 ± 0.000** | 0.000 ± 0.000** | 0.000 ± 0.000** |
| Model | 2 | / | 0.160 ± 0.010 | 0.150 ± 0.020 | 0.170 ± 0.032 | 0.164 ± 0.024 | 0.144 ± 0.040 | 0.134 ± 0.033 |
| Adalimumab | 2 | 8 | 0.042 ± 0.035** | 0.071 ± 0.016* | 0.092 ± 0.014* | 0.094 ± 0.011* | 0.088 ± 0.025* | 0.090 ± 0.031* |
| mPEG-SC₂₀ₖ-HM-3 | 2 | 20 | 0.101 ± 0.027* | 0.092 ± 0.054** | 0.125 ± 0.035* | 0.116 ± 0.034* | 0.086 ± 0.008* | 0.091 ± 0.004* |

| | | Dosage | Times | | | |
|---|---|---|---|---|---|---|
| Groups | N | (mg/kg) | 24 h | 48 h | 72 h | 96 h |
| Normal | 2 | / | 0.000 ± 0.000** | 0.000 ± 0.000** | 0.001 ± 0.001** | 0.001 ± 0.001** |
| Model | 2 | / | 0.136 ± 0.042 | 0.146 ± 0.013 | 0.142 ± 0.009 | 0.114 ± 0.021 |
| Adalimumab | 2 | 8 | 0.132 ± 0.006 | 0.152 ± 0.074 | 0.150 ± 0.066 | 0.133 ± 0.061 |
| mPEG-SC₂₀ₖ-HM-3 | 2 | 20 | 0.107 ± 0.010* | 0.105 ± 0.010* | 0.101 ± 0.016* | 0.076 ± 0.006** |

vs. model group,
*p < 0.05,
**p < 0.01.

TABLE 14
The impact of mPEG-SC₂₀ₖ-HM-3 on acute λ-carrageenan inflammation of left toe swelling changes in TNF-luc mouse
(mean ± SD, N = 2)

| | | Dosage | Times | | | | | |
|---|---|---|---|---|---|---|---|---|
| Groups | N | (mg/kg) | 1 h | 3 h | 5 h | 7 h | 9 h | 12 h |
| Normal | 2 | / | 0.000 ± 0.000** | 0.000 ± 0.000** | 0.000 ± 0.000** | 0.000 ± 0.000** | 0.000 ± 0.000** | 0.000 ± 0.000** |
| Model | 2 | / | 0.166 ± 0.023 | 0.166 ± 0.050 | 0.162 ± 0.034 | 0.134 ± 0.037 | 0.122 ± 0.020 | 0.132 ± 0.031 |
| Adalimumab | 2 | 8 | 0.094 ± 0.054** | 0.115 ± 0.018* | 0.150 ± 0.028 | 0.126 ± 0.014 | 0.126 ± 0.021 | 0.114 ± 0.025 |
| mPEG-SC₂₀ₖ-HM-3 | 2 | 20 | 0.132 ± 0.054 | 0.114 ± 0.025* | 0.146 ± 0.003 | 0.174 ± 0.065 | 0.124 ± 0.014 | 0.113 ± 0.027 |

| | | Dosage | Times | | | |
|---|---|---|---|---|---|---|
| Groups | N | (mg/kg) | 24 h | 48 h | 72 h | 96 h |
| Normal | 2 | / | 0.000 ± 0.000** | 0.000 ± 0.000** | 0.001 ± 0.001** | 0.001 ± 0.000** |
| Model | 2 | / | 0.124 ± 0.027 | 0.142 ± 0.031 | 0.144 ± 0.034 | 0.142 ± 0.014 |
| Adalimumab | 2 | 8 | 0.235 ± 0.134 | 0.264 ± 0.122 | 0.238 ± 0.113 | 0.174 ± 0.102 |
| mPEG-SC₂₀ₖ-HM-3 | 2 | 20 | 0.136 ± 0.011 | 0.126 ± 0.031 | 0.094 ± 0.034** | 0.084 ± 0.040** |

vs, *p < 0.05,
**p < 0.01.

TABLE 15
The changes of fluorescence intensity in λ-carrageenan induced actue inflammation mice
(mean ± SD, N = 2)

| | | Dosage | Fluorescence intensity (×10⁵) | | | | |
|---|---|---|---|---|---|---|---|
| Groups | N | (mg/kg) | 0 h | 2 h | 4 h | 8 h | 12 h |
| Model | 2 | / | 29.094 ± 1.720 | 21.159 ± 4.222 | 21.791 ± 2.546 | 15.150 ± 1.833 | 16.677 ± 1.961 |
| Adalimumab | 2 | 8 | 18.048 ± 4.770 | 18.480 ± 4.887 | 20.209 ± 2.261 | 25.703 ± 2.668 | 22.396 ± 5.065 |
| mPEG-SC20k-HM-3 | 2 | 20 | 10.851 ± 3.014 | 13.880 ± 5.266 | 11.926 ± 2.119 | 19.367 ± 3.408 | 18.653 ± 9.093 |

| | | Dosage | Fluorescence intensity (×10⁵) | | |
|---|---|---|---|---|---|
| Groups | N | (mg/kg) | 24 h | 48 h | 72 h |
| Model | 2 | / | 13.252 ± 2.224 | 13.909 ± 2.104 | 13.521 ± 1.273 |
| Adalimumab | 2 | 8 | 13.891 ± 0.853 | 14.765 ± 3.580 | 19.868 ± 3.924 |
| mPEG-SC₂₀ₖ-HM-3 | 2 | 20 | 12.654 ± 2.390 | 14.037 ± 6.238 | 13.402 ± 1.171 |

vs. model group, *p < 0.05,
**p < 0.01.

### Embodiment 11

### Effect on Chronic Inflammation Caused by Adjuvant-Induced Arthritis in Transgenic TNF-luc Mice

### I. Test Objectives

To study whether mPEG-SC₂₀ₖ-HM-3 can inhibit adjuvant-induced chronic inflammation in Balb/C transgenic TNF-luc mice and relieve inflammation.

### II. Drug Tested

Name: mPEG-SC₂₀ₖ-HM-3; provider: Synthesized by our laboratory; batch No.: 140408.

### III. Control Drugs

1. Name: Adalimumab; manufacturer: AbbVie; specification: 40 mg/0.8 mL, batch No.: 28388LX01

2. Name: 0.9% sodium chloride injection; manufacturer: Cisen Pharmaceutical Co., Ltd.; specification: 500 mL:4.5 g, batch No.: 1303207021

### IV. Grouping and Dose Setting

TABLE 16
Dose regimen

| | | | | | Administration | | |
|---|---|---|---|---|---|---|---|
| | | Induction | | Administrated | time and drug | Fluorescein | |
| Groups | N | method | Test | dose | delivery | delivery | Detection |
| Normal | 2 | / | Saline | 100 μL/20 g | once per two | 2 mg/20 g | Small |
| | | | | | days sc. | ip. | animal in |
| Model | 2 | CFA 50 μL | Saline | 100 μL/20 g | once per two | 2 mg/20 g | vivo |
| | | | | | days sc. | ip. | imaging |
| Positive | 2 | CFA 50 μL | Adalimumab | 8 mg/kg | once per 15 | 2 mg/20 g | instrument |
| | | | | | days sc. | ip. | 430 nm/535 |
| Test | 2 | CFA 50 μL | mPEG- | 20 mg/kg | once per two | 2 mg/20 g | nm |
| | | | SC₂₀ₖ-HM-3 | | days sc. | ip. | |

### V. Drug Preparation Method

1. Positive drug group: 16 μL of Adalimumab original medicine solution is added to 984 μL of normal saline injection to reach a concentration of 0.8 mg/mL, and the administration volume is 0.2 mL/20 g of body weight.

2. Test administration group: 0.02133 g (20 mg is required) of mPEG-SC₂₀ₖ-HM-3 powder is weighed and put into 10 mL of normal saline to reach a concentration of 2 mg/mL, and the administration volume is 0.2 mL/20 g of body weight.

Note: Sterile normal saline is used for preparation before each administration.

### VI. Animals

Source, strain: SPF BALB/c transgenic TNF-luc mice with genetic background, provided by Model Animal Research Center of Nanjing University (laboratory animal production license No.: SCXK (Su) 2015-0001; laboratory animal quality certificate: 201500002). Number of the using of Laboratory Animal. SYXK (Jun) 2012-0049.

Age: 6-7 weeks at the time of purchase and 7-8 weeks at the beginning of administration;

weight: (18±22) g at the time of purchase and (20±24) g at the beginning of administration; gender: half male and half female, n=2.

### VII. Inflammatic Agent

| CFA | Chondrex, Inc. | Specification: | Batch |
|---|---|---|---|
| | | 10 mg/mL, | No.: |
| | | 5 mL | 130477 |

(Preparation method: uniformly mix before use, store at 4° C. in the absence of light)

### VIII. Luminescence Reagent

VivoGlo™ Luciferin, In Vivo Grade; manufacturer: Promega Corporation; specification: 50 mg; batch No.:124359179

### IX. Test Instruments

Electronic balance, BT25S Sartorius Scientific Instrument (Beijing) Co., Ltd.

ChemBase CBS-CJ-1FD super clean bench, Suzhou AIRTECH Air Technology Co., Ltd.

1 mL of Sterile disposable syringe (0.5×20 RWLB), Jiangsu Changcheng Medical Equipment Co., Ltd.

0-150 type vernier caliper, Taiwan Southwest Jiahua Precision Measuring Tool Co., Ltd.

Carestream MI small animal in vivo imager (Bruker)

### X. Test Method

### 1. Establishment of a Model of Adjuvant-Induced Arthritis in Transgenic TNF-luc Mice

8 animals are randomly divided into 4 groups (n=2), a normal group, a model group, a positive drug group and a test group. On day 0, the initial left and right ankle thicknesses, initial left and right foot palm thicknesses and initial left and right toe thicknesses of each group of mice are measured. On day 0, 50 μL/mouse complete Freund's adjuvant is injected into the bottom of the left toe of each group of mice, and the treatment is started after secondary inflammation appears in the right foot of each group of mice on day 17. On day 17, the normal group and the model group are hypodermically injected with 0.9% normal saline (0.2 mL/20 g of body weight), once every 2 days, 7 times in total. The positive drug Adalimumab group is hypodermically injected with Adalimumab (8 mg/kg of body weight), once every 15 days, once in total. The test group is hypodermically injected with mPEG-SC₂₀ₖ-HM-3 (20 mg/kg of body weight), once every 2 days, 7 times in total. The treatment lasts for 15 days. Then, medication is stopped for observation for one week, and the mice are killed one week later.

### 2. Arthritis Index Evaluation

### 2.1 Paw Swelling Degree

On day 0, the initial ankle widths, foot palm widths and toe thicknesses of left and right feet are measured with a vernier caliper as initial values. Every 2 days, the left and right ankle widths, foot palm widths and toe thicknesses of each group of mice are measured separately with the vernier caliper, and compared with the initial values to calculate the changes in the swelling degree.

### 2.2 Joint Index Score

The joints of the mouse paw are observed with naked eyes, and the joints are scored every 2 days from day 17. Scoring is performed in terms of level 0 to level 4, 5 levels in total. The evaluation method is shown in the following table:

TABLE 17
Joint index score standard

| Joint | |
|---|---|
| index | |
| score | Pathological characteristics |
| 0 | No erythema or redness |
| 1 | Mild erythema or swelling, one of the former / toe joints have |
| | erythema or swelling |
| 2 | More than one toe appears erythema or swelling |
| 3 | Foot swelling of ankle or wrist joint |
| 4 | All feet, including the ankle joint swelling |
| | The four feet of the mice were scored and the highest score |
| | was 16. |

### 2.3 Systemic Joint Score

After the secondary inflammation appears, systemic scoring is performed every 2 days.

TABLE 18
Systemic clinical score standard
Systemic clinical score
Hind foot: 0 points = no swelling, 1 points = a hind foot swelling,
2 points = two hind foot swelling
Front foot: 0 points = no swelling, 1 points = a front foot swelling,
2 points = two front foot swelling
Ears: 0 points = no redness symptoms and nodules, 1 points =
one ear redness symptoms or nodules, 2 points = two
ears with red symptoms and nodules
Nose: 0 points = no swelling, 1 points = obvious swelling
Tail: 0 points = no nodules, 1 points = nodules; the highest
score was 8 points

### 3. In Vivo Imaging Evaluation of Mice

On day 17 and day 38, in vivo imaging evaluation is performed on mice in each group. After the mice are lightly anesthetized with ether, the mice are fixed within 5-20 min and put into an in vivo imager, the imager is covered with an instrument lid, on-fluro and om-photo names in the desktop imaging interface of the computer are clicked, and the expose button is clicked to complete the fluorescence and white light photos of mice in vivo imaging.

(1) Imaging principle: Fluorescein substrate is injected intraperitoneally, and binds to luciferase carried on TNF-α protein to catalyze luminescence. Through the Luciferase fluorescent reporter group on TNF, the change of the fluorescence area in mice is observed, and the expression of TNF-α is evaluated, so as to evaluate the impact of mPEG-SC₂₀ₖ-HM-3 on the expression of TNF-α.

(2) Operation steps: Start the Carestreama in vivo imager, open the MI software, click the Capture button, and set photographing conditions. In the on-flour fluorescence mode, set the parameters X Binning. 2 pixels, Y Binning. 2 pixels; Illumination Source: Multi-wavelength; f-stop: 2.50; field of view (FOV) (mm): 120; focal plane (mm): Tray. Excitation filter: 430 nm, emission filter: 535 nm; exposure time: 30s. Click the Expose button to photograph fluorescence. Under the om-photo name, set the parameters X Binning. 0 pixels, Y Binning. 0 pixels; Illumination Source: None; f-stop: 2.50; FOV (mm): 120; focal plane (mm): Tray. Excitation filter: 0 nm, emission filter: 0 nm; exposure time: 0.175 s. Click the Expose button to photograph white light.

(3) Image processing and output: Analyze and adjust photos of fluorescence function imaging and white light structure imaging to the best, and the FOV is consistent. Click Window→Tie, Display→Invert, Image Display→overlay→Transparency for image stacking. In the image output process, in a navigation panel, click Annotation→Width/Heigh→Scale Analysis Image for setting, click Add Annotation Bar for adding a title, click Add Intensity Scale for adding a ruler, and set Horizontal, Size, Style, Number of Significant Dig, and Horizontal/Vertical Length. Output a picture in Tiff format.

### XI. Immunohistochemical Analysis

Changes of expression of TNF-α and IL-6 are analyzed by HE staining and immunohistochemistry in the ankle joint, spleen and thymus of the mice. Observation and photographing are performed under the FOVs of ×200 and ×400 times respectively, 5 FOVs are taken randomly for each picture, and data statistics is performed.

### XII. Data Statistics

Test results are expressed by x±s, and the t test between each group and the control group is performed by using SPSS 11.0 software. * means p<0.05, and ** means p<0.01.

### XIII. Results and Discussions

The results of swelling degree of left ankle of mice shows (in Tab 17, FIG. 40) that mPEG-SC₂₀ₖ-HM-3 can effectively inhibit swelling of left ankle of transgenic TNF-luc mice. Compared with those of the positive drug Adalimumab group (8 mg/kg) on day 28 (0.211±0.028) and day 31 (0.202±0.037), the swelling degrees of left ankle of mice in the mPEG-SC₂₀ₖ-HM-3 group (20 mg/kg) on day 28 and day 31 are (0.265±0.028) and (0.212±0.010) respectively, and there is no significant difference in inhibitory effect, indicating that the two have almost the same therapeutic effect. Compared with those of the model group on day 28 (0.298±0.058) and day 31 (0.288±0.070), the inhibitory effects of the mPEG-SC₂₀ₖ-HM-3 group (20 mg/kg) are extremely significantly different (**p<0.01).

The results of swelling degree of right ankle of mice (in Tab 18, FIG. 41) show that mPEG-SC₂₀ₖ-HM-3 can effectively inhibit swelling of right ankle of transgenic TNF-luc mice. On day 22, day 25, day 28 and day 31, the swelling degrees of the right ankle of mice in the model group are (0.026±0.012), (0.027±0.003), (0.030±0.010) and (0.030±0.020), respectively, and on day 22, day 25, day 28 and day 31, the swelling degrees of the right ankle of mice in the positive drug Adalimumab group (8 mg/kg) are (0.006±0.006), (0.006±0.008), (0.009±0.001) and (0.005±0.010), respectively. Compared with those of the model group, the swelling degrees of the right ankle of mice in the mPEG-SC₂₀ₖ-HM-3 group (20 mg/kg) on day 22, day 25, day 28 and day 31 are (0.013±0.008), (0.012±0.006), (0.021±0.013) and (0.012±0.006), respectively, and the inhibitory effect is significantly different (*p<0.05). Compared with those of the positive drug Adalimumab group (8 mg/kg), the swelling degrees of the right ankle of mice in the mPEG-SC₂₀ₖ-HM-3 group (20 mg/kg) have a significant difference on day 22, day 25, day 28 and day 31 (*p<0.05), but the inhibitory effect is not as good as that of the positive drug.

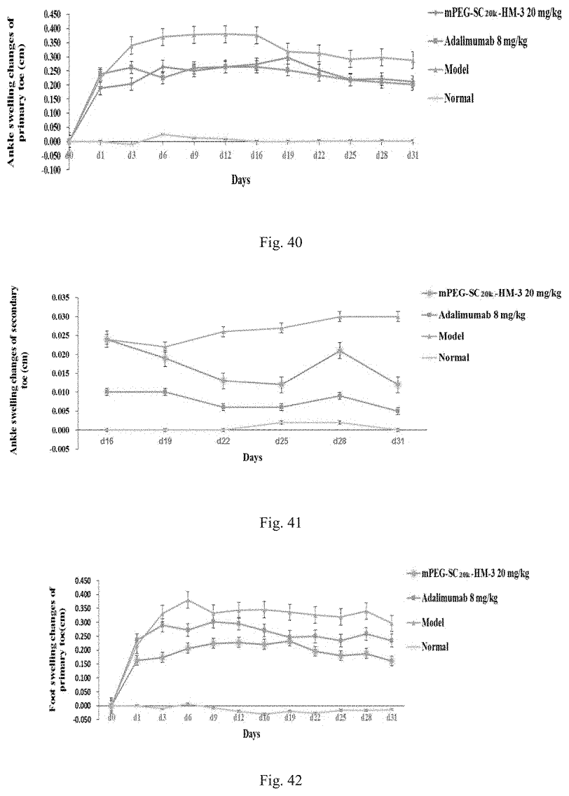

The results of swelling degree of left foot palm of mice shows (in Tab 19, FIG. 42) that mPEG-SC₂₀ₖ-HM-3 can effectively inhibit swelling of left foot palm of transgenic TNF-luc mice. Compared with those of the model group, the inhibitory effects of the mPEG-SC₂₀ₖ-HM-3 group (20 mg/kg) are extremely significantly different on day 19, day 22, day 25, day 28 and day 31 (**p<0.01). Compared with those of the positive drug Adalimumab group (8 mg/kg), the swelling degrees of the left foot palm of mice in the mPEG-SC₂₀ₖ-HM-3 (20 mg/kg) on day 19, day 22, day 25, day 28 and day 31 are (0.233±0.047), (0.196±0.071), (0.180±0.074), (0.188±0.045) and (0.161±0.055), respectively, the inhibitory effects are significantly different (*p<0.05), and on day 31, the inhibitory effect is better than that of the Adalimumab group (8 mg/kg).

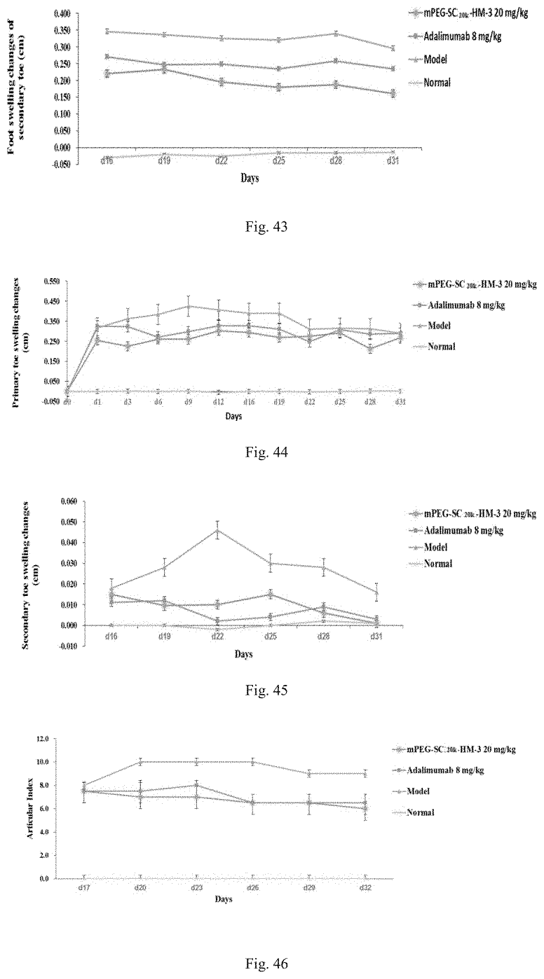

The results of swelling degree of right foot palm of mice shows (in Tab 20, FIG. 43) that mPEG-SC₂₀ₖ-HM-3 can effectively inhibit swelling of right foot palm of transgenic TNF-luc mice. Compared with those of the model group, the inhibitory effects of the mPEG-SC₂₀ₖ-HM-3 group (20 mg/kg) have a significant difference on day 22 (*p<0.05), and its inhibitory effects are extremely significantly different on day 25, day 28 and day 31 (**p<0.01). Compared with those of the positive drug Adalimumab group (8 mg/kg), the swelling degrees of the right foot palm of mice in the mPEG-SC₂₀ₖ-HM-3 (20 mg/kg) on day 25, day 28 and day 31 are (0.001±0.013), (0.004±0.020) and (0.003±0.018), respectively, the inhibitory effects are significantly different (*p<0.05), and on day 25, day 28 and day 31, the inhibitory effects are better than those of the Adalimumab group (8 mg/kg).

The results of swelling degree of left toe of mice shows (in Tab 21, FIG. 44) that mPEG-SC₂₀ₖ-HM-3 can effectively inhibit swelling of left toe of transgenic TNF-luc mice. Compared with those of the model group, the inhibitory effects of the mPEG-SC₂₀ₖ-HM-3 group (20 mg/kg) are extremely significantly different on day 19, day 22 and day 28 (**p<0.01). Compared with those of the positive drug Adalimumab group (8 mg/kg), the swelling degrees of the left toe of mice in the mPEG-SC₂₀ₖ-HM-3 (20 mg/kg) on day 19, day 22 and day 28 are (0.269±0.092), (0.276±0.141) and (0.213±0.154), respectively, the inhibitory effects are significantly different (*p<0.05), and on day 19 and day 28, the inhibitory effects are better than those of the Adalimumab group (8 mg/kg).

The results of swelling degree of right toe of mice (in Tab 22, FIG. 45) show that mPEG-SC₂₀ₖ-HM-3 can effectively inhibit swelling of right toe of transgenic TNF-luc mice. Compared with those of the model group, the inhibitory effects of the mPEG-SC₂₀ₖ-HM-3 group (20 mg/kg) are extremely significantly different on day 19, day 22, day 28 and day 31 (**p<0.01). Compared with those of the positive drug Adalimumab group (8 mg/kg), the swelling degrees of the right toe of mice in the mPEG-SC₂₀ₖ-HM-3 (20 mg/kg) on day 19, day 22, day 28 and day 31 are (0.009±0.012), (0.010±0.014), (0.006±0.020) and (0.001±0.030), respectively, and the inhibitory effects are significantly different (*p<0.05).

The results of mouse joint index score shows (in Tab 23, FIG. 46) that mPEG-SC₂₀ₖ-HM-3 can effectively inhibit the joint index score of transgenic TNF-luc mice. Compared with those of the model group, mouse joint index scores of the mPEG-SC₂₀ₖ-HM-3 group (20 mg/kg) on day 20, day 23, day 26, day 29 and day 32 are (7.0±1.4), (7.0±1.4), (6.5±0.7), (6.5±0.7) and (6.0±0.0) respectively, and the inhibitory effects are significantly different (*p<0.05), wherein on day 26 and day 32, the inhibitory effects are extremely significantly different (**p<0.01). Compared with that of the positive drug Adalimumab group (8 mg/kg), the mouse joint index score of the mPEG-SC₂₀ₖ-HM-3 group (20 mg/kg) on day 32 is (6.0±0.0), and the inhibitory effects are significantly different (*p<0.05).

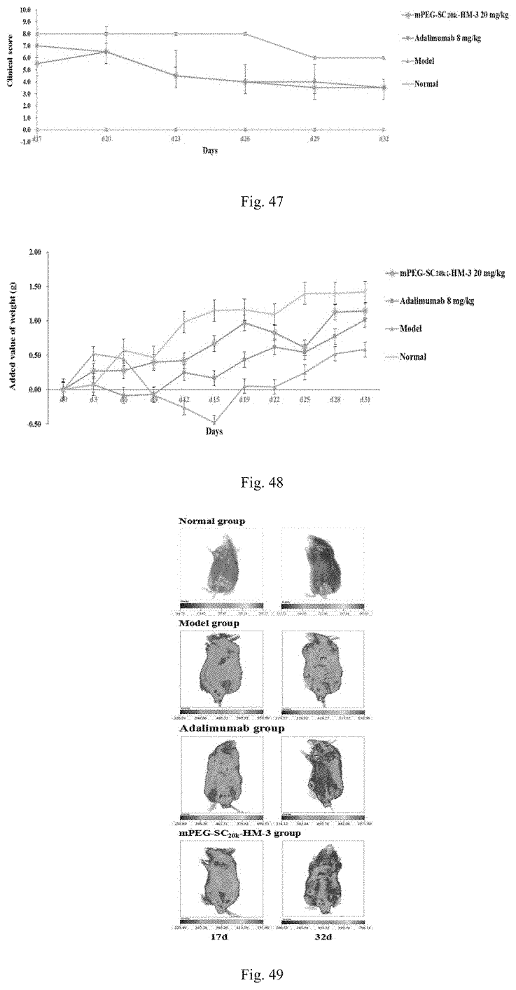

The results of systemic score of mice (in Tab 24, FIG. 47) show that mPEG-SC₂₀ₖ-HM-3 can effectively inhibit systemic score of transgenic TNF-luc mice. Compared with those of the model group, systemic scores of mice of the mPEG-SC₂₀ₖ-HM-3 group (20 mg/kg) on day 20, day 23, day 26, day 29 and day 32 are (6.5±0.7), (4.5±0.7), (4.0±0.0), (3.5±0.7) and (3.5±0.7) respectively, and the inhibitory effects are significantly different (*p<0.05), wherein on day 26 and day 29, the inhibitory effects are extremely significantly different (**p<0.01). Compared with that of the positive drug Adalimumab group (8 mg/kg), the systemic score of mice of the mPEG-SC₂₀ₖ-HM-3 group (20 mg/kg) on day 29 is (3.5±0.7), and the inhibitory effect is significantly different (*p<0.05).

The results of mouse weight changes (in Tab 25, FIG. 48) show that mPEG-SC₂₀ₖ-HM-3 can increase the weight of transgenic TNF-luc mice. Compared with that of the positive drug Adalimumab group (8 mg/kg), the weight gain of mice of the mPEG-SC₂₀ₖ-HM-3 group (20 mg/kg) has a significant difference (*p<0.05). Compared with that of the model group, the weight gain of mice of the mPEG-SC₂₀ₖ-HM-3 group (20 mg/kg) is extremely significantly different (**p<0.01).

In vivo imaging results of mice (in Tab 26, FIG. 49) show that the in vivo fluorescence area intensity of mice in each group is the highest on day 17 under observation with naked eyes, indicating that the established model of adjuvant-induced arthritis is successful. The in vivo fluorescence densities (10⁶) in the normal group, the model group, the Adalimumab group (8 mg/kg) and the mPEG-SC₂₀ₖ-HM-3 group (20 mg/kg) are (1.50±0.47), (57.72±1.97), (46.75±3.46) and (41.6±1.68), respectively. On day 32, the in vivo fluorescence densities (10⁶) in the normal group, the model group, the Adalimumab group (8 mg/kg) and the mPEG-SC₂₀ₖ-HM-3 group (20 mg/kg) are (1.78±0.6), (52.45±5.08), (26.45±2.66) and (31.96±1.17), respectively. Compared with that of the model group, the in vivo fluorescence area of mice of the mPEG-SC₂₀ₖHM-3 decreases, with an extremely significant difference (***p<0.001), indicating that mPEG-SC₂₀ₖ-HM-3 can effectively inhibit the expression of TNF-α in mice. Compared with that of the positive drug Adalimumab group, the in vivo fluorescence area of mice of the mPEG-SC₂₀ₖ-HM-3 group (20 mg/kg) decreases, is lower than that of the positive drug Adalimumab group and is extremely significantly different (***p<0.001), indicating that the mPEG-SC₂₀ₖ-HM-3 group can effectively inhibit the expression of TNF-α in mice, and the inhibitory effect is better than that of the positive drug Adalimumab group.

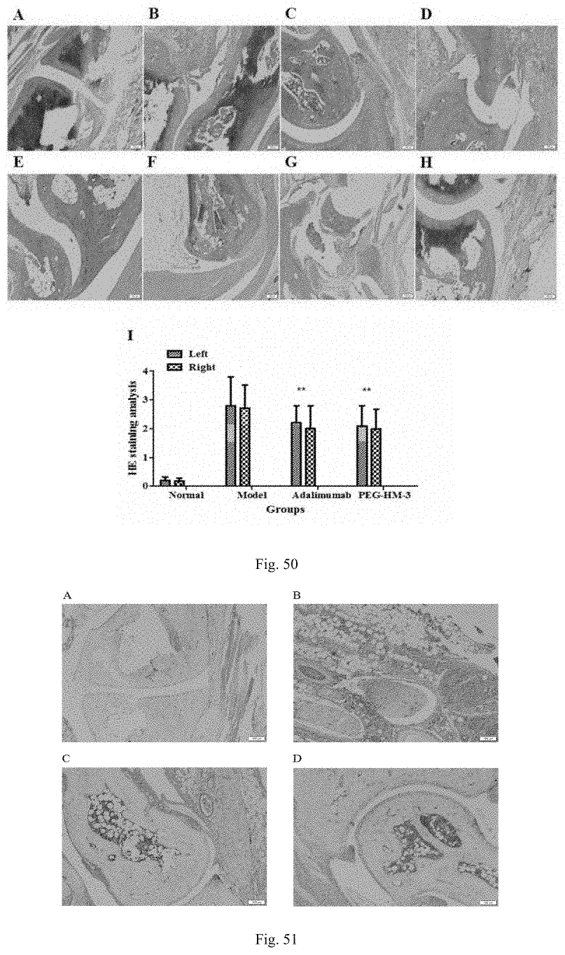

HE staining results show that (in FIG. 50), the knee joint structure of mice of the normal group is intact, and has no pathological symptoms. In the model group, hyperplasia of synovial membrane in the joint cavity of mice is serious, and cartilage and facial bone invasion and pathological changes of pannus appear. Compared with hyperplasia of synovial membrane and infiltration of mice in the positive drug Adalimumab group (8 mg/kg), hyperplasia of synovial membrane of the mPEG-SC₂₀ₖ-HM-3 group (20 mg/kg) is lighter, and its infiltration is less. Compared with those of the model group, pathological symptoms of the Adalimumab group and the mPEG-SC₂₀ₖ-HM-3 group are relieved to some extent (**p<0.01). The relief of symptoms of the mPEG-SC₂₀ₖ-HM-3 group is equivalent to that of the positive drug Adalimumab group, and the two have no significant difference.

### Immunohistochemical Analysis Results:

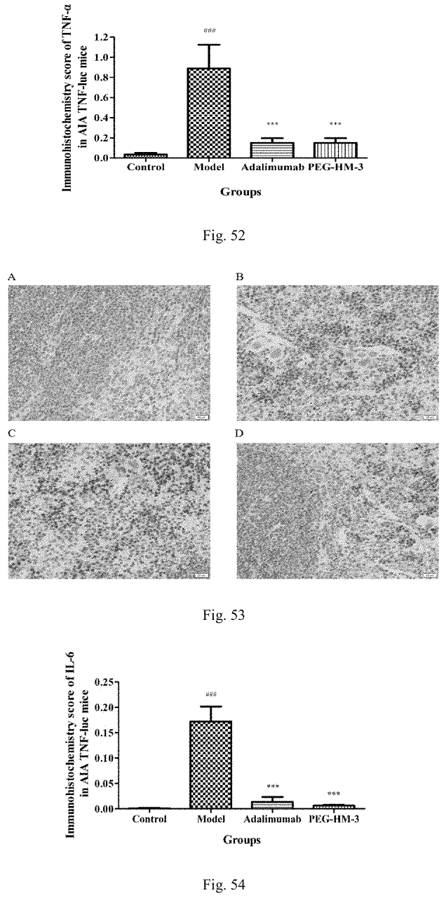

(1) Immunohistochemical analysis of TNF-α in ankle joint cavity of TNF-luc mice Results show that (in FIG. 51 and FIG. 52), immunohistochemical scores of TNF-α in the joint cavity of the normal group, the model group, the Adalimumab group (8 mg/kg) and the mPEG-SC₂₀ₖ-HM-3 group (20 mg/kg) are (0.036±0.016), (0.828±0.255), (0.246±0.110) and (0.149±0.047), respectively. Compared with that of the model group, the immunohistochemical scores of the Adalimumab group and the mPEG-SC₂₀ₖHM-3 group are extremely significantly different (***p<0.001); and compared with that of the positive drug Adalimumab group, the immunohistochemical score of the mPEG-SC₂₀ₖ-HM-3 group has no significant difference. (2) Immunohistochemical analysis of IL-6 in spleen of TNF-luc mice Results show that (in FIG. 53 and FIG. 54), immunohistochemical scores of IL-6 in spleen of the normal group, the model group, the Adalimumab group (8 mg/kg) and the mPEG-SC₂₀ₖ-HM-3 group (20 mg/kg) are (0.001±0.001), (0.172±0.029), (0.013±0.010) and (0.006±0.002), respectively. Compared with that of the model group, the immunohistochemical scores of the Adalimumab group and the mPEG-SC₂₀ₖ-HM-3 group are extremely significantly different (***p<0.001); and compared with that of the positive drug Adalimumab group, the immunohistochemical score of the mPEG-SC₂₀ₖHM-3 group has no significant difference.

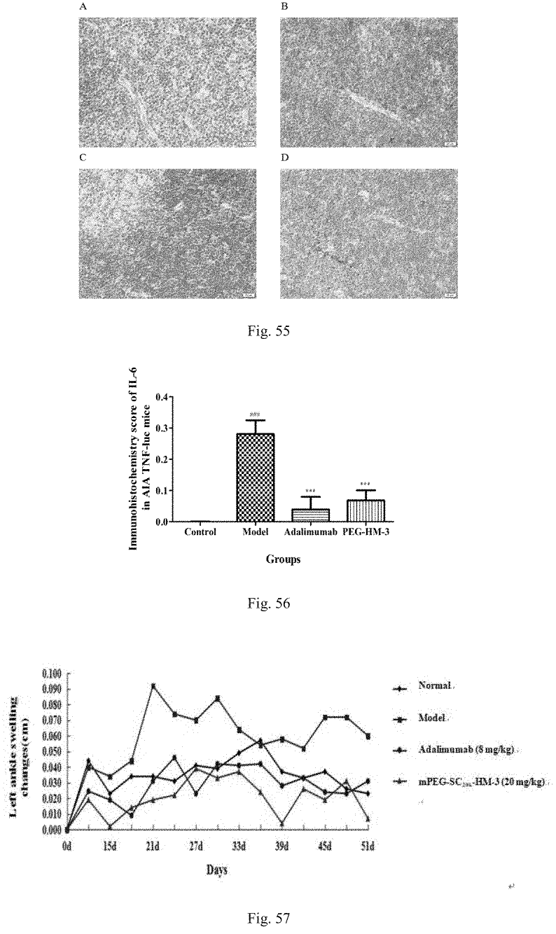

(3) Immunohistochemical analysis of IL-6 in thymus of TNF-luc mice Results show that (in FIG. 55 and FIG. 56), immunohistochemical scores of IL-6 in thymus of the normal group, the model group, the Adalimumab group (8 mg/kg) and the mPEG-SC₂₀ₖ-HM-3 group (20 mg/kg) are (0.000±0.000), (0.281±0.044), (0.039±0.041) and (0.068±0.033), respectively. Compared with that of the model group, the immunohistochemical scores of the Adalimumab group and the mPEG-SC₂₀ₖHM-3 group are extremely significantly different (***p<0.001); and compared with that of the positive drug Adalimumab group, the immunohistochemical score of the mPEG-SC₂₀ₖ-HM-3 group has no significant difference.

### XIV. Conclusion

According to statistical test results, mPEG-SC₂₀ₖ-HM-3 (20 mg/kg) can effectively inhibit chronic inflammation caused by adjuvant-induced arthritis in transgenic TNF-luc mice. It can effectively inhibit the (primary) ankle swelling degree, foot palm swelling degree and toe swelling degree of the left foot caused by adjuvant-induced arthritis in transgenic TNF-α mice. It can effectively inhibit the (secondary) ankle swelling degree, foot palm swelling degree and toe swelling degree of the right foot. It can reduce systemic score and arthritis index score of transgenic TNF-luc mice. It can effectively reduce the expression of inflammatory factor TNF-α of adjuvant-induced arthritis in transgenic TNF-luc mice, and the comprehensive evaluation effect in treating chronic inflammation caused by adjuvant-induced arthritis in transgenic TNF-luc mice in the later stage is better than that of the Adalimumab positive control group.

TABLE 17
The changes of left ankle swelling in AIA transgenic TNF-luc mice (mean ± SD, n = 2)

| | | Dosage | Time | | | | | |
|---|---|---|---|---|---|---|---|---|
| Groups | N | (mg/kg) | 0 d | 1 d | 3 d | 6 d | 9 d | 12 d |
| Normal | 2 | / | 0.000 ± 0.000 | 0.000 ± 0.000** | 0.010 ± 0.000** | 0.024 ± 0.004** | 0.012 ± 0.001** | 0.008 ± 0.002** |
| Model | 2 | / | 0.000 ± 0.000 | 0.224 ± 0.102 | 0.340 ± 0.118 | 0.370 ± 0.090 | 0.378 ± 0.110 | 0.380 ± 0.160 |
| Adalimumab | 2 | 8 | 0.000 ± 0.000 | 0.239 ± 0.033 | 0.262 ± 0.071 | 0.226 ± 0.023 | 0.260 ± 0.020 | 0.264 ± 0.018* |
| mPEG-SC₂₀ₖ- | 2 | 20 | 0.000 ± 0.000 | 0.188 ± 0.018 | 0.204 ± 0.074 | 0.265 ± 0.031 | 0.251 ± 0.029 | 0.265 ± 0.028 |
| HM-3 | | | | | | | | |

| | Time | | | | | |
|---|---|---|---|---|---|---|
| Groups | 16 d | 19 d | 22 d | 25 d | 28 d | 31 d |
| Normal | 0.000 ± 0.00** | 0.000 ± 0.000** | 0.002 ± 0.000** | 0.002 ± 0.001** | 0.002 ± 0.001** | 0.002 ± 0.001** |
| Model | 0.376 ± 0.082 | 0.318 ± 0.056 | 0.312 ± 0.100 | 0.292 ± 0.180 | 0.298 ± 0.058 | 0.288 ± 0.070 |
| Adalimumab | 0.265 ± 0.021 | 0.254 ± 0.018* | 0.235 ± 0.013** | 0.218 ± 0.028* | 0.211 ± 0.028** | 0.202 ± 0.037** |
| mPEG-SC₂₀ₖ- | 0.274 ± 0.020 | 0.297 ± 0.005* | 0.252 ± 0.006* | 0.219 ± 0.013* | 0.221 ± 0.007** | 0.212 ± 0.010** |
| HM-3 | | | | | | |

vs model group,
*p < 0.05,
**p < 0.01.

TABLE 18
The changes of right ankle swelling in AIA transgenic TNF-luc mice
(mean ± SD, n = 2)

| | | Dosage | | | | | | |
|---|---|---|---|---|---|---|---|---|
| Groups | N | (mg/kg) | 16 d | 19 d | 22 d | 25 d | 28 d | 31 d |
| Normal | 2 | / | 0.000 ± 0.000** | 0.000 ± 0.000** | 0.000 ± 0.000** | 0.002 ± 0.001** | 0.002 ± 0.001** | 0.000 ± 0.000** |
| Model | 2 | / | 0.024 ± 0.002 | 0.022 ± 0.004 | 0.026 ± 0.012 | 0.027 ± 0.003 | 0.030 ± 0.010 | 0.030 ± 0.020 |
| Adalimumab | 2 | 8 | 0.010 ± 0.006 | 0.010 ± 0.014* | 0.006 ± 0.006** | 0.006 ± 0.008** | 0.009 ± 0.001** | 0.005 ± 0.010** |
| mPEG- | 2 | 20 | 0.024 ± 0.011 | 0.019 ± 0.009 | 0.013 ± 0.008* | 0.012 ± 0.006* | 0.021 ± 0.013 | 0.012 ± 0.006* |
| SC₂₀ₖ-HM-3 | | | | | | | | |

vs model group,
*p < 0.05,
**p < 0.01.

TABLE 19
The changes of left foot palm in AIA transgenic TNF-luc mice (mean ± SD, n = 2)

| | | Dosage | Time | | | | | |
|---|---|---|---|---|---|---|---|---|
| Groups | N | (mg/kg) | 0 d | 1 d | 3 d | 6 d | 9 d | 12 d |
| Normal | 2 | / | 0.000 ± 0.000 | 0.000 ± 0.000** | 0.010 ± 0.000** | 0.006 ± 0.000** | 0.008 ± 0.003** | 0.020 ± 0.000** |
| Model | 2 | / | 0.000 ± 0.000 | 0.216 ± 0.046 | 0.332 ± 0.106 | 0.380 ± 0.085 | 0.334 ± 0.120 | 0.343 ± 0.112 |
| Adalimumab | 2 | 8 | 0.000 ± 0.000 | 0.236 ± 0.062 | 0.289 ± 0.016 | 0.272 ± 0.057 | 0.302 ± 0.003 | 0.295 ± 0.021 |
| mPEG-SC₂₀ₖ- | 2 | 20 | 0.000 ± 0.000 | 0.163 ± 0.052 | 0.174 ± 0.088 | 0.207 ± 0.055 | 0.224 ± 0.062 | 0.228 ± 0.054 |
| HM-3 | | | | | | | | |

| | Time | | | | | |
|---|---|---|---|---|---|---|
| Groups | 16 d | 19 d | 22 d | 25 d | 28 d | 31 d |
| Normal | 0.030 ± 0.005** | 0.020 ± 0.001** | 0.026 ± 0.000** | 0.016 ± 0.002** | 0.016 ± 0.000** | 0.014 ± 0.000** |
| Model | 0.346 ± 0.078 | 0.336 ± 0.049 | 0.326 ± 0.028 | 0.320 ± 0.130 | 0.340 ± 0.200 | 0.296 ± 0.16 |
| Adalimumab | 0.271 ± 0.064 | 0.247 ± 0.078** | 0.249 ± 0.126** | 0.234 ± 0.040** | 0.258 ± 0.042** | 0.235 ± 0.061* |
| mPEG-SC₂₀ₖ- | 0.221 ± 0.041 | 0.233 ± 0.047** | 0.196 ± 0.071** | 0.180 ± 0.074** | 0.188 ± 0.045** | 0.161 ± 0.055** |
| HM-3 | | | | | | |

vs model group,
*p < 0.05,
**p < 0.01.

TABLE 20
The changes of right foot palm in AIA transgenic TNF-luc mice (mean ± SD, n = 2)

| | | Dosage | Times | | | | | |
|---|---|---|---|---|---|---|---|---|
| Groups | N | (mg/kg) | 16 d | 19 d | 22 d | 25 d | 28 d | 31 d |
| Normal | 2 | / | 0.030 ± 0.003 | 0.020 ± 0.002* | 0.026 ± 0.004 | 0.016 ± 0.007 | 0.016 ± 0.001 | 0.014 ± 0.002* |
| Model | 2 | / | 0.022 ± 0.042 | 0.006 ± 0.001 | 0.014 ± 0.003 | 0.022 ± 0.012 | 0.016 ± 0.002 | 0.024 ± 0.003 |
| Adalimumab | 2 | 8 | 0.010 ± 0.006 | 0.029 ± 0.001 | 0.004 ± 0.003* | 0.013 ± 0.016* | 0.008 ± 0.006* | 0.010 ± 0.003* |
| mPEG- | 2 | 20 | 0.007 ± 0.007 | 0.010 ± 0.011 | 0.003 ± 0.016* | 0.001 ± 0.013** | 0.004 ± 0.020** | 0.003 ± 0.018** |
| SC₂₀ₖ-HM-3 | | | | | | | | |

vs model group,
*p < 0.05,
**p < 0.01.

TABLE 21
The changes of left toe in AIA trasgenic TNF-luc mice (mean ± SD, n = 2)

| | | Dosage | Time | | | | | |
|---|---|---|---|---|---|---|---|---|
| Groups | N | (mg/kg) | 0 d | 1 d | 3 d | 6 d | 9 d | 12 d |
| Normal | 2 | / | 0.000 ± 0.000 | 0.000 ± 0.000** | 0.002 ± 0.000** | 0.000 ± 0.000** | 0.001 ± 0.000** | 0.004 ± 0.001** |
| Model | 2 | / | 0.000 ± 0.000 | 0.318 ± 0.001 | 0.364 ± 0.140 | 0.384 ± 0.066 | 0.426 ± 0.122 | 0.406 ± 0.308 |
| Adalimumab | 2 | 8 | 0.000 ± 0.000 | 0.326 ± 0.028 | 0.323 ± 0.086 | 0.272 ± 0.076 | 0.298 ± 0.122 | 0.328 ± 0.079 |
| mPEG-SC₂₀ₖ- | 2 | 20 | 0.000 ± 0.000 | 0.255 ± 0.027 | 0.225 ± 0.106 | 0.261 ± 0.126 | 0.260 ± 0.156 | 0.303 ± 0.103 |
| HM-3 | | | | | | | | |

| | Time | | | | | |
|---|---|---|---|---|---|---|
| Groups | 16 d | 19 d | 22 d | 25 d | 28 d | 31 d |
| Normal | 0.000 ± 0.000** | 0.000 ± 0.000** | 0.002 ± 0.000** | 0.000 ± 0.000** | 0.002 ± 0.001** | 0.001 ± 0.000** |
| Model | 0.390 ± 0.048 | 0.390 ± 0.076 | 0.310 ± 0.061 | 0.316 ± 0.133 | 0.312 ± 0.106 | 0.290 ± 0.100 |
| Adalimumab | 0.328 ± 0.065 | 0.311 ± 0.033* | 0.248 ± 0.136* | 0.309 ± 0.058 | 0.285 ± 0.075* | 0.290 ± 0.023 |
| mPEG-SC₂₀ₖ- | 0.294 ± 0.092 | 0.269 ± 0.092** | 0.276 ± 0.141* | 0.292 ± 0.107* | 0.213 ± 0.154** | 0.270 ± 0.127 |
| HM-3 | | | | | | |

vs model group,
*p < 0.05,
**p < 0.01.

TABLE 22
The changes of right toe in AIA transgenic TNF-luc mice (mean ± SD, n = 2)

| | | Dosage | Time | | | | | |
|---|---|---|---|---|---|---|---|---|
| Groups | N | (mg/kg) | 16 d | 19 d | 22 d | 25 d | 28 d | 31 d |
| Normal | 2 | / | 0.000 ± 0.000* | 0.000 ± 0.000** | 0.002 ± 0.000** | 0.000 ± 0.000** | 0.002 ± 0.001** | 0.001 ± 0.000** |
| Model | 2 | / | 0.018 ± 0.012 | 0.028 ± 0.008 | 0.046 ± 0.028 | 0.030 ± 0.010 | 0.028 ± 0.014 | 0.016 ± 0.009 |
| Adalimumab | 2 | 8 | 0.011 ± 0.033 | 0.012 ± 0.037* | 0.002 ± 0.042** | 0.004 ± 0.040** | 0.009 ± 0.035** | 0.003 ± 0.021** |
| mPEG- | 2 | 20 | 0.015 ± 0.021 | 0.009 ± 0.012** | 0.010 ± 0.014** | 0.015 ± 0.027* | 0.006 ± 0.020** | 0.001 ± 0.030** |
| SC₂₀ₖ-HM-3 | | | | | | | | |

vs model group,
*p < 0.05,
**p < 0.01.

TABLE 23
The changes of joint index in AIA transgenic TNF-luc mice (mean ± SD, n = 2)

| | | Dosage | Time | | | | | |
|---|---|---|---|---|---|---|---|---|
| Groups | N | (mg/kg) | 17 d | 20 d | 23 d | 26 d | 29 d | 32 d |
| Normal | 2 | / | 0.00 ± 0.00** | 0.00 ± 0.00** | 0.00 ± 0.00** | 0.00 ± 0.00** | 0.00 ± 0.00** | 0.00 ± 0.00** |
| Model | 2 | / | 8.00 ± 0.70 | 10.00 ± 0.70 | 10.00 ± 0.70 | 10.00 ± 0.70 | 9.00 ± 0.70 | 9.00 ± 0.70 |
| Adalimumab | 2 | 8 | 7.50 ± 0.70 | 7.50 ± 0.70* | 8.00 ± 0.00* | 6.50 ± 0.70** | 6.50 ± 0.70* | 6.50 ± 0.70* |
| mPEG- | 2 | 20 | 7.50 ± 0.70 | 7.00 ± 1.40* | 7.00 ± 1.40* | 6.50 ± 0.70** | 6.50 ± 0.70* | 6.00 ± 0.00** |
| SC₂₀ₖ-HM-3 | | | | | | | | |

vs model group,
*p < 0.05,
**p < 0.01.

TABLE 24
The changes of whole body score in AIA transgenic TNF-luc mice (mean ± SD, n = 2)

| | | Dosage | Time | | | | | |
|---|---|---|---|---|---|---|---|---|
| Groups | N | (mg/kg) | 17 d | 20 d | 23 d | 26 d | 29 d | 32 d |
| Normal | 2 | / | 0.00 ± 0.00** | 0.00 ± 0.00** | 0.00 ± 0.00** | 0.00 ± 0.00** | 0.00 ± 0.00** | 0.00 ± 0.00** |
| Model | 2 | / | 8.00 ± 0.70 | 8.00 ± 0.70 | 8.00 ± 0.70 | 8.00 ± 1.40 | 6.00 ± 0.70 | 6.00 ± 0.70 |
| Adalimumab | 2 | 8 | 7.00 ± 1.40 | 6.50 ± 2.10* | 4.50 ± 2.10* | 4.00 ± 1.40** | 4.00 ± 1.40* | 3.50 ± 0.70* |
| mPEG- | 2 | 20 | 5.50 ± 0.70 | 6.50 ± 0.70* | 4.50 ± 0.70* | 4.00 ± 0.00** | 3.50 ± 0.70** | 3.50 ± 0.7* |
| SC₂₀ₖ-HM-3 | | | | | | | | |

vs model group,
*p < 0.05,**
**p < 0.01.

TABLE 25
The changes of weight in AIA transgenic TNF-luc mice (mean ± SD, n = 2)

| | | | Time | | | | |
|---|---|---|---|---|---|---|---|
| Groups | N | Dosage (mg/kg) | 0 d | 3 d | 6 d | 9 d | 12 d |
| Normal | 2 | / | 0.00 ± 0.00 | 0.07 ± 0.01* | 0.57 ± 0.10 | 0.47 ± 0.30* | 0.98 ± 0.28** |
| Model | 2 | / | 0.00 ± 0.00 | 0.52 ± 0.04 | 0.45 ± 0.32 | 0.08 ± 0.21 | 0.26 ± 0.40 |
| Adalimumab | 2 | 8 | 0.00 ± 0.00 | 0.07 ± 0.15 | 0.09 ± 0.54 | 0.07 ± 0.25 | 0.25 ± 0.83 |
| mPEG- | 2 | 20 | 0.00 ± 0.00 | 0.27 ± 0.06 | 0.28 ± 0.25 | 0.40 ± 0.39 | 0.42 ± 0.66 |
| SC₂₀ₖ-HM-3 | | | | | | | |
| | | Time | | | | | |
| | Groups | 15 d | 19 d | 22 d | 25 d | 28 d | 31 d |
| | Normal | 1.15 ± 0.04** | 1.16 ± 0.20** | 1.09 ± 0.50** | 1.40 ± 0.34** | 1.40 ± 0.60** | 1.42 ± 0.38** |
| | Model | 0.48 ± 0.54 | 0.05 ± 0.63 | 0.04 ± 0.24 | 0.25 ± 0.14 | 0.52 ± 0.28 | 0.58 ± 0.35 |
| | Adalimumab | 0.16 ± 0.66 | 0.44 ± 0.60* | 0.62 ± 0.48** | 0.55 ± 0.02 | 0.77 ± 0.33 | 1.02 ± 0.98** |
| | mPEG- | 0.67 ± 1.06 | 0.97 ± 1.09** | 0.82 ± 0.57** | 0.61 ± 0.72* | 1.13 ± 0.18** | 1.15 ± 0.40** |
| | SC₂₀ₖ-HM-3 | | | | | | |

vs model group,
*p < 0.05,
**p < 0.01.

TABLE 26
The changes of fluorescence intensity in AIA transgenic TNF-luc
mice (mean ± SD, n = 2)

| | | Dosage | Fluorescence intensity (×10⁵) | |
|---|---|---|---|---|
| Groups | N | (mg/kg) | 17 d | 32 d |
| Normal | 2 | / | 1.502 ± 0.047*** | 1.78 ± 0.60*** |
| Model | 2 | / | 57.720 ± 1.972 | 52.455 ± 5.081 |
| Adalimumab | 2 | 8 | 46.746 ± 3.457* | 26.446 ± 2.659*** |
| mPEG- | 2 | 20 | 41.604 ± 6.080** | 31.956 ± 1.170** |
| SC₂₀ₖ-HM-3 | | | | |

vs model group,
*p < 0.05,
**p < 0.01.

### Embodiment 12

### Effect on Chronic Inflammation Caused by Collagen Type II-Induced Arthritis in Transgenic TNF-luc Mice

### I. Test Objectives

To study whether mPEG-SC₂₀ₖ-HM-3 can inhibit chronic inflammation caused by collagen type II-induced arthritis in Balb/C transgenic TNF-luc mice and relieve inflammation.

### II. Drug Tested

Name: mPEG-SC₂₀ₖ-HM-3

Provider: Synthesized by our laboratory; batch No.: 140408.

### III. Control Drugs

1. Name: Adalimumab; manufacturer: AbbVie; specification: 40 mg/0.8 mL, batch No.: 28388LX01

2. Solvent: 0.9% sodium chloride injection; manufacturer: Cisen Pharmaceutical Co., Ltd.; specification: 500 mL:4.5 g; batch No.: 1303207021

### IV. Grouping and Dose Setting

TABLE 27
Dose regimen

| | | Induction | | Induced | | Administration | Fluorescein | |
|---|---|---|---|---|---|---|---|---|
| Groups | N | methods | Treatment | dose | | time and drug delivery | delivery | Detection |
| Normal | 2 | / | Saline | 100 | μL/20 g | once per 2 days, 7 times s.c. | 2 mg/20 g | Small animal in vivo imaging |
| | | | | | | | i.p. | instrument 430 nm/535 nm |
| Model | 2 | CFA/IFA + | Saline | 100 | μL/20 g | once per 2 days, 7 times s.c. | 2 mg/20 g | |
| | | II-type | | | | | i.p. | |
| Positive | 2 | collagen | Adalimumab | 8 | mg/kg | once per 15 days, 1 times s.c. | 2 mg/20 g | |
| | | emulsion | | | | | i.p. | |
| Test | 2 | | mPEG-SC₂₀k- | 20 | mg/kg | once per 2 days, 7 times s.c. | 2 mg/20 g | |
| | | | HM-3 | | | | i.p. | |

### V. Drug Preparation Method

1. 0.1M acetic acid solution 57.2 μL of glacial acetic acid (d1.05, M60.05) is pipetted using a pipette into 10 mL of normal saline, to obtain the 0.1 M acetic acid solution.

2. Collagen type II solution 0.00482 g (4 mg is required) of collagen type II solution is weighed using an electronic balance and dissolved in 1 mL of glacial acetic acid solution with a concentration of 0.1 moL/L in an EP tube, and the solution stands overnight at 4° C. to obtain collagen acetic acid solution with a concentration of 4 mg/mL.

Emulsification: On day 0, 1 mL of CFA containing Mycobacterium tuberculosis H37 RA with a concentration of 4 mg/mL is fully emulsified with the same volume of CII acetic acid solution at a ratio of 1:1 to obtain 2 mg/mL emulsifier for primary immunization.

3. Collagen type II solution 0.00417 g (4 mg is needed) of collagen type II solution is weighed using an electronic balance and dissolved in 1 mL of glacial acetic acid solution with a concentration of 0.1 moL/L in an EP tube, and the solution stands overnight at 4° C. to obtain collagen acetic acid solution with a concentration of 4 mg/mL.

Emulsification: On day 21, IFA is fully emulsified with the same volume of CII acetic acid solution at a ratio of 1:1 to obtain 2 mg/mL emulsifier for secondary immunization.

4. Adalimumab (8 mg/kg): In a super clean bench, 16 μL of Adalimumab original medicine solution is pipetted using a pipette to 984 μL of normal saline injection to reach a concentration of 0.8 mg/mL, and the administration volume is 0.2 mL/20 g of body weight.

5. mPEG-SC₂₀ₖ-HM-3 (20 mg/kg): 0.02099 g (20 mg is required) of freeze-dried powder is weighed using an electronic balance and added into 10 mL of normal saline to reach a concentration of 2 mg/mL, and the solution is filtered in the super clean bench, with the administration volume being 0.2 mL/20 g of body weight (note: sterile normal saline is used for preparation before each administration).

### VI. Animals

Source, strain: SPF BALB/c transgenic TNF-luc mice with genetic background, provided by Model Animal Research Center of Nanjing University (laboratory animal production license No.: SCXK (Su) 2015-0001; laboratory animal quality certificate: 201500002). Number of the using of Laboratory Animal: SYXK (Jun) 2012-0049.

Age: 6-7 weeks at the time of purchase and 7-8 weeks at the beginning of administration;

weight: (18±22) g at the time of purchase and (20±24) g at the beginning of administration; gender: half male and half female, n=2.

### VII. Inflammatic Agent

| Chicken collagen | Sigma-Aldrich | Specification: 25 mg |
|---|---|---|
| type II (CII) | Inc. | Batch No.: 9007-34-5 |
| CFA | Chondrex, Inc. | Specification: 10 mg/mL, 5 mL |
| | | Batch No.: 130477 |
| Incomplete | Chondrex, Inc. | Specification: 4 mg/mL, 5 mL |
| Freund's adjuvant | | Batch No.: 140490 |
| (IFA) | | |

### VIII. Luminescence Reagent

VivoGlo™ Luciferin, In Vivo Grade; Promega Corporation; specification: 50 mg;

batch No.:124359179

### IX. Test Instruments

Electronic balance, BT25S Sartorius Scientific Instrument (Beijing) Co., Ltd.

ChemBase CBS-CJ-1FD super clean bench, Suzhou AIRTECH Air Technology Co., Ltd.

1 mL of Sterile disposable syringe (0.5×20 RWLB), Jiangsu Changcheng Medical Equipment Co., Ltd.

0-150 type vernier caliper, Taiwan Southwest Jiahua Precision Measuring Tool Co., Ltd.

Small animal in vivo imager, Carestream Health, Inc.

### X. Test Method

1. A model of Chronic Inflammation Caused by Collagen Type II Arthritis in Transgenic TNF-luc Mice is Established.

8 animals are randomly divided into 4 groups (n=2), a normal group, a model group, a positive drug group and a test group. On day 0, for the primary immunization, mice are hypodermically injected with 50 μL collagen emulsifier (containing CII 100 μg/ mouse) at the tail root of mice for sensitization. On day 0, initial ankle widths, initial foot palm widths and initial toe thicknesses of left and right feet of mice in each group are measured separately.

After day 21, the same dosage of emulsifier is used for second immunization at the tail root. After the symptoms such as redness and swelling of joints appear on the four toes of mice, continuous administration is performed for 15 days. The normal group and the model group are separately hypodermically injected with 0.9% normal saline (0.2 mL/20 g of body weight), once every 2 days, 7 times in total. The positive drug Adalimumab group is hypodermically injected with Adalimumab (8 mg/kg of body weight), once every 15 days, once in total. The test treatment group is hypodermically injected with mPEG-SC₂₀ₖ-HM-3 (20 mg/kg of body weight), once every 2 days, 7 times in total.

### 2. Arthritis Index Evaluation

### 2.1 Paw Swelling Degree

Every 2 days, the thickness of the ankle, the width of the foot palm and the thickness of the toes of each group of mice are measured with a vernier caliper.

Formula for calculating the swelling degree is as follows:

Swelling degree of left ankle (cm)=swelling thickness of left ankle on day n of the experiment−measured initial thickness of left ankle.

Swelling degree of right ankle (cm)=swelling thickness of right ankle on day n of the experiment−measured initial thickness of right ankle.

### 2.2 Joint Index Score

The joints of the mouse paw are observed with naked eyes, and the joints are scored every 2 days from day 30. Scoring is performed in terms of level 0 to level 4, 5 levels in total. 0=no erythema or swelling; 1=slight erythema or swelling, with erythema or swelling on one of the front/hind toe joints; 2=erythema or swelling on more than one toe; 3=swelling of paw beneath the ankle or wrist joint; 4=swelling of all paws including the ankle joint. Four paws of the mice are scored separately, and the highest score is 16 points.

### 2.3 Systemic Score

After the secondary inflammation appears, systemic scoring is performed every 2 days, and the scoring standards are as follows:

Hind foot: no swelling=0 point, swelling of one hind foot=1 point, swelling of two hind feet=2 points.

Front foot: no swelling=0, swelling of one front foot=1 point, swelling of two front feet=2 points.

Ears: no redness symptoms and nodules=0 point, redness symptoms or nodules of one ear=1 point, red symptoms and nodules of two ears=2 points.

Nose: no swelling=0 point, obvious swelling=1 point.

Tail: no nodule=0 point, nodules=1 point; the highest score is 8 points.

### 3. In Vivo Imaging Evaluation of Mice

On day 30, day 35, day 40 and day 45, in vivo imaging evaluation is performed mice in each group. After the mice are lightly anesthetized with ether, the mice are fixed within 5-20 min and put into an in vivo imager, the imager is covered with an instrument lid, on-fluro and om-photo names in the desktop imaging interface of the computer are clicked, and the expose button is clicked to complete the fluorescence and white light photos of mice in vivo imaging. The specific imaging principle, operation steps, image processing and output are the same as those in the previous embodiment.

### XI. Immunohistochemical Analysis

Determining criteria: The experimental results are determined based on the proportion of positive cells in all tissue cells and the staining intensity of positive cells: A: scoring is performed based on the number of chromogenic cells, 1 point is given when the proportion of the number of positive cells is less than ⅓, 2 points are given when the proportion of the number of positive cells is ⅓-⅔, and 3 points are given when the proportion of the number of positive cells is greater than or equal to ⅔. B: Based on the color depth of cells, 0 point is given when no cells have positive reaction, 1 point is given when cells are light yellow, 2 points are given when the cells are light brown, and 3 points are given when the cells are dark brown. Point product=A×B. The result is expressed in point product.

### XII. Data Statistics

Test results are expressed by x±s, and the t test between each group and the control group is performed by using SPSS 11.0 software. * means p<0.05, and ** means p<0.01.

### XIII. Results and Discussions

1. The results of swelling degree changes of left ankle of mice shows (in Tab 28, FIG. 57) that mPEG-SC₂₀ₖ-HM-3 can effectively inhibit swelling of left ankle of transgenic TNF-luc mice. Compared with those of the positive drug Adalimumab (8 mg/kg), the swelling degrees of left ankle of mice of the mPEG-SC₂₀ₖ-HM-3 group (20 mg/kg) on day 33, day 45 and day 48 are (0.007±0.013), (0.011±0.004) and (0.001±0.021), respectively, and the inhibitory effects have no significant difference; however, on day 39 and day 51, the swelling degrees of left ankle of mice are (0.026±0.023) and (0.023±0.007) respectively, and the inhibitory effects are slightly weaker than those of the positive drug group. Compared with those of the model group, the swelling degrees of left ankle of mice of the mPEG-SC₂₀ₖ-HM-3 group (20 mg/kg) on day 33, day 45 and day 48 are (0.007±0.013), (0.011±0.004) and (0.001±0.021), respectively, and the inhibitory effects are extremely significantly different (**p<0.01).

2. The results of swelling degree of right ankle changes of mice (in Tab 29, FIG. 58) show that mPEG-SC₂₀ₖ-HM-3 can effectively inhibit swelling of right ankle of transgenic TNF-luc mice. Compared with those of the positive drug Adalimumab group (8 mg/kg), the swelling degrees of right ankle of mice of the mPEG-SC₂₀ₖ-HM-3 group (20 mg/kg) on day 36, day 39, day 42, day 45, day 48 and day 51 are (0.006±0.008), (0.001±0.008), (0.001±0.007), (0.000±0.006), (0.001±0.007) and (0.005±0.007) respectively, and the inhibitory effects are significantly different (*p<0.05). Compared with those of the model group, the inhibitory effects of the mPEG-SC₂₀ₖ-HM-3 group (20 mg/kg) are extremely significantly different (**p<0.01).

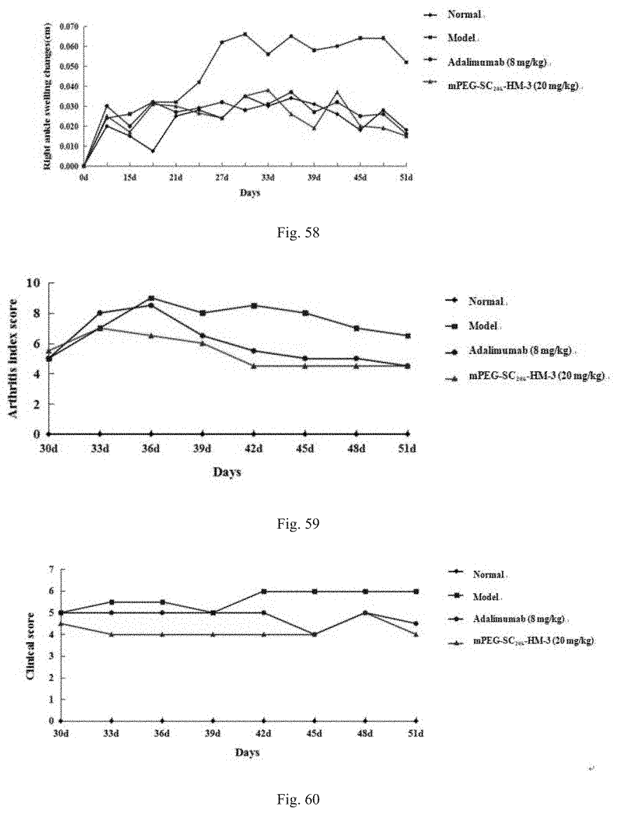

3. The results of mouse joint index score shows that (in Tab 30, FIG. 59), mPEG-SC₂ok-HM-3 can effectively inhibit the joint index score of transgenic TNF-luc mice. Compared with the positive drug Adalimumab group (8 mg/kg), mouse joint index scores of the mPEG-SC₂₀ₖ-HM-3 group (20 mg/kg) on day 36, day 39, day 42, day 45, day 48 and day 51 are (6.50±0.71), (6.00±0.00), (4.50±0.7), (4.50±0.71), (4.50±0.71) and (4.50±0.71), respectively, and the inhibitory effects are significantly different (*p<0.05); mPEG-SC₂₀ₖ-HM-3 inhibits the joint index almost all the time, and the inhibitory effects are better than those of the positive drug group. Within one week after the stopping of medication, the inhibitory effects on day 48 and day 51 are still good; and compared with those of the model group, the inhibitory effects of the mPEG-SC₂₀ₖ-HM-3 group (20 mg/kg) are extremely significantly different (**p<0.01).

4. The results of systemic score of mice (in Tab 31, FIG. 60) show that mPEG-SC₂₀ₖ-HM-3 can effectively inhibit systemic score of transgenic TNF-luc mice. Compared with the positive drug Adalimumab group (8 mg/kg), mouse joint index scores of the mPEG-SC₂₀ₖ-HM-3 group (20 mg/kg) on day 33, day 36, day 39, day 42, day 45 and day 51 are (4.00±0.00), (4.00±0.00), (4.00±0.00), (4.00±0.00), (4.00±0.00) and (4.00±0.00), respectively, and the inhibitory effects are extremely significantly different (**p<0.01); mPEG-SC₂₀ₖ-HM-3 inhibits the joint index almost all the time, and the inhibitory effects are better than those of the positive drug group. Within one week after the stopping of medication, the inhibitory effects on day 51 are still good; and compared with those of the model group, the inhibitory effects of the mPEG-SC₂₀ₖ-HM-3 group (20 mg/kg) are extremely significantly different (**p<0.01).

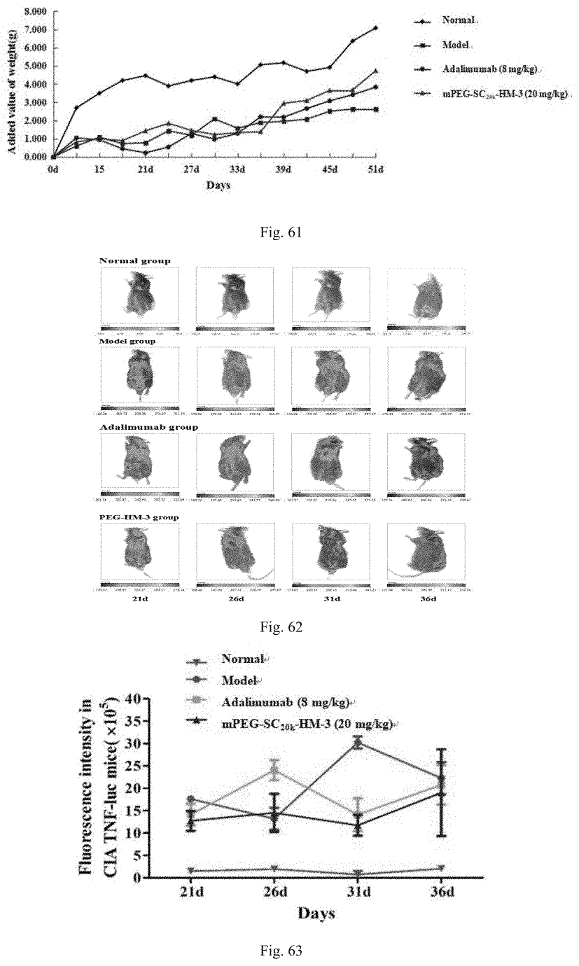

5. The results of mouse weight changes (in Tab 32, FIG. 61) show that mPEG-SC₂₀ₖ-HM-3 can increase the weight of transgenic TNF-luc mice. Compared with that of the positive drug Adalimumab group (8 mg/kg), the weight gain of mice of the mPEG-SC₂₀ₖ-HM-3 group (20 mg/kg) has a significant difference (*p<0.05). Compared with that of the model group, the weight gain of mice of the mPEG-SC₂₀ₖ-HM-3 group (20 mg/kg) is obvious and extremely significantly different (**p<0.01).

6. In vivo imaging results of mice (in FIG. 62, FIG. 63) show that the in vivo fluorescence area intensity of mice in each group is the highest on day 21 and day 31, indicating that the established model of chronic inflammation caused by collagen type II arthritis is successful. On day 36, the in vivo fluorescence densities (10⁵) in the normal group, the model group, the Adalimumab group (8 mg/kg) and the mPEG-SC₂₀ₖ-HM-3 group (20 mg/kg) are (1.50±0.47), (22.27±3.55), (20.76±4.43) and (19.02±9.69), respectively. Compared with that of the model group, the in vivo fluorescence area of mice of the mPEG-SC₂₀ₖ-HM-3 group decreases significantly, with an extremely significant difference (***p<0.001), indicating that mPEG-SC₂₀ₖ-HM-3 can effectively inhibit the expression of TNF-α in mice. Compared with that of the positive drug Adalimumab group, the in vivo fluorescence area of mice of the mPEG-SC₂₀ₖ-HM-3 group is equivalent and has no significant difference, indicating that the mPEG-SC₂₀ₖ-HM-3 group can effectively inhibit the expression of TNF-α in mice, and the inhibitory effect is equivalent to that of the positive drug Adalimumab group.

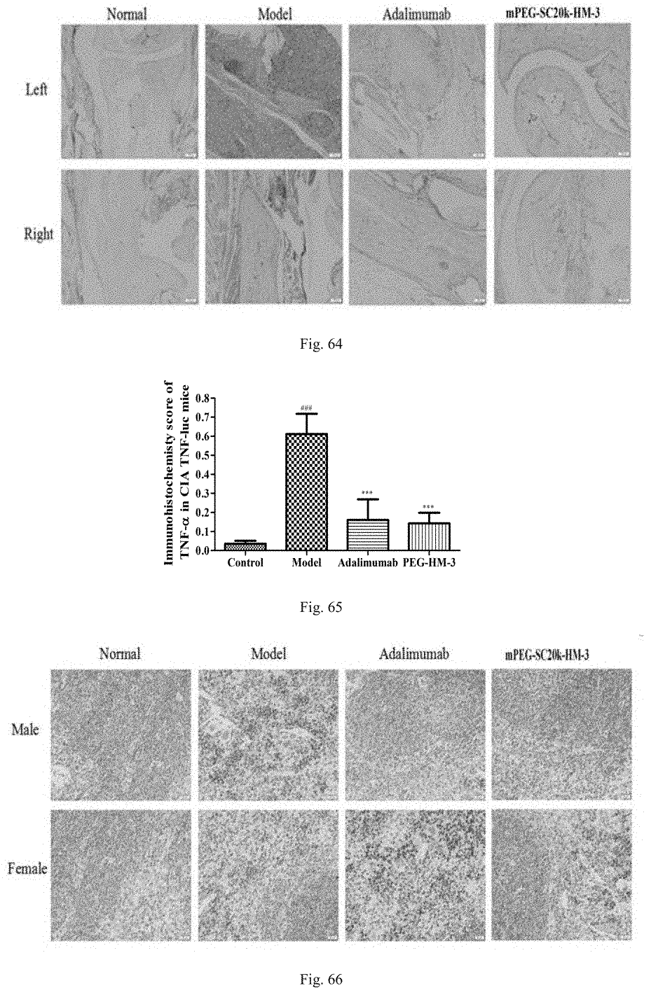

7. Immunohistochemical analysis results (FIG. 64 to FIG. 69) show immunohistochemical analysis of TNF-α in ankle joint cavity of TNF-luc mice. Results show that immunohistochemical scores of TNF-α in the joint cavity of the normal group, the model group, the Adalimumab group (8 mg/kg) and the mPEG-SC₂₀ₖ-HM-3 group (20 mg/kg) are (0.037±0.015), (0.611±0.107), (0.160±0.109) and (0.143±0.055), respectively. Compared with that of the model group, the immunohistochemical scores of the Adalimumab group and the mPEG-SC₂₀ₖ-HM-3 group are extremely significantly different (***p<0.001); and compared with that of the positive drug Adalimumab group, the immunohistochemical score of the mPEG-SC₂₀ₖ-HM-3 group has no significant difference Immunohistochemical analysis of IL-6 in spleen of TNF-luc mice Results show that immunohistochemical scores of IL-6 in spleen of the normal group, the model group, the Adalimumab group (8 mg/kg) and the mPEG-SC₂₀ₖ-HM-3 group (20 mg/kg) are (0.001±0.001), (0.172±0.018), (0.008±0.003) and (0.017±0.009), respectively. Compared with that of the model group, the immunohistochemical scores of the Adalimumab group and the mPEG-SC₂₀ₖ-HM-3 group are extremely significantly different (***p<0.001); and compared with that of the positive drug Adalimumab group, the immunohistochemical score of the mPEG-SC₂₀ₖ-HM-3 group has no significant difference.

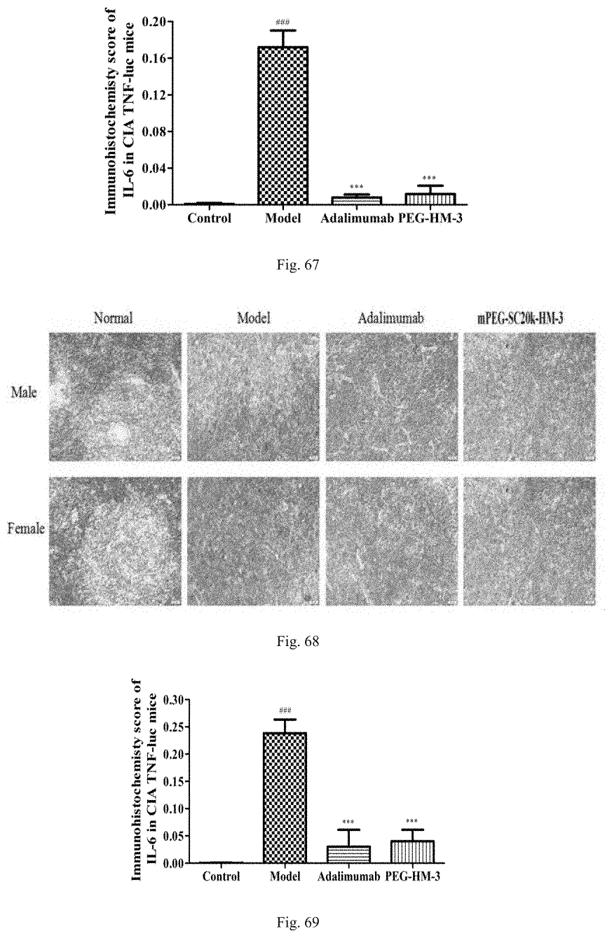

Immunohistochemical analysis of IL-6 in thymus of TNF-luc mice Results show that (in FIG. 67 to FIG. 69), immunohistochemical scores of IL-6 in thymus of the normal group, the model group, the Adalimumab group (8 mg/kg) and the mPEG-SC₂₀ₖ-HM-3 (20 mg/kg) are (0.000±0.001), (0.238±0.025), (0.030±0.031) and (0.040±0.021), respectively. Compared with that of the model group, the immunohistochemical scores of the Adalimumab group and the mPEG-SC₂₀ₖ-HM-3 group are extremely significantly different (***p<0.001); and compared with that of the positive drug Adalimumab group, the immunohistochemical score of the mPEG-SC₂₀ₖ-HM-3 group has no significant difference.

### XIV. Conclusion

According to statistical test results, mPEG-SC₂₀ₖ-HM-3 (20 mg/kg) can effectively inhibit chronic inflammation caused by collagen type II arthritis in transgenic TNF-luc mice. It can effectively inhibit the swelling degree of left ankle of collagen type II arthritis in transgenic TNF-α mice, effectively inhibit swelling degree of right ankle, and reduce systemic scores and arthritis index scores of transgenic TNF-α mice in the whole process, and especially within one week after the stopping of medication, its inhibitory effect is still very good. It is speculated that the effect of mPEG-SC₂₀ₖ-HM-3 in vivo is persistent and delayed, and its action mechanism is different from that of Adalimumab. HE staining results of ankle joint show that mPEG-SC₂₀ₖ-HM-3 reduces bone invasion and the formation of pannus. Immunohistochemical results show that mPEG-SC₂₀ₖ-HM-3 decreases the expression of TNF-α in ankle joint, and decreases the expression of IL-6 in spleen and thymus. During in vivo imaging, mPEG-SC₂₀ₖ-HM-3 can effectively reduce the expression of inflammatory factor TNF-α of adjuvant-induced arthritis in transgenic TNF-luc mice, indicating that mPEG-SC₂₀ₖ-HM-3 can inhibit the expression of TNF-α in an animal arthritis model. Therefore, the comprehensive evaluation of mPEG-SC₂₀ₖ-HM-3 on the alleviation of arthritis inflammation is equivalent to or better than that of the Adalimumab positive control group (8 mg/kg).

### XV. The Test Results are Summarized as Follows

TABLE 28
The impact of mPEG-SC₂₀ₖ-HM-3 on CIA chronic inflammation of left ankle swelling changes in TNF-luc mouse (mean ± SD, N = 2, cm)

| Groups | N | 0 d | 12 d | 15 d | 18 d | 21 d | 24 d | 27 d | 30 d |
|---|---|---|---|---|---|---|---|---|---|
| Normal | 2 | 0.000 ± 0.000 | 0.014 ± 0.006 | 0.007 ± 0.010 | 0.004 ± 0.006 | 0.004 ± 0.023** | 0.001 ± 0.007** | 0.011 ± 0.007* | 0.009 ± |
| | | | | | | | | | 0.004** |
| Model | 2 | 0.000 ± 0.000 | 0.010 ± 0.010 | 0.004 ± 0.014 | 0.014 ± 0.000 | 0.062 ± 0.012 | 0.044 ± 0.004 | 0.040 ± 0.006 | 0.054 ± |
| | | | | | | | | | 0.012 |
| Adalimumab | 2 | 0.000 ± 0.000 | 0.006 ± 0.050 | 0.011 ± 0.072 | 0.021 ± 0.058 | 0.001 ± 0.041 | 0.016 ± 0.048 | 0.007 ± 0.049 | 0.012 ± |
| (8 mg/kg) | | | | | | | | | 0.045** |
| mPEG- | 2 | 0.000 ± 0.000 | 0.011 ± 0.004 | 0.028 ± 0.023 | 0.016 ± 0.008 | 0.011 ± 0.010 | 0.008 ± 0.006 | 0.009 ± 0.004 | 0.003 ± |
| SC₂₀ₖ-HM-3 | | | | | | | | | 0.007** |
| (20 mg/kg) | | | | | | | | | |
| | | Groups | 33 d | 36 d | 39 d | 42 d | 45 d | 48 d | 51 d |
| | | Normal | 0.019 ± 0.001* | 0.027 ± 0.001 | 0.007 ± 0.001 | 0.003 ± 0.007 | 0.007 ± 0.001* | 0.004 ± 0.000** | 0.007 ± |
| | | | | | | | | | 0.010* |
| | | Model | 0.034 ± 0.018 | 0.024 ± 0.028 | 0.028 ± 0.008 | 0.022 ± 0.008 | 0.042 ± 0.006 | 0.042 ± 0.004 | 0.030 ± |
| | | | | | | | | | 0.014 |
| | | Adalimumab | 0.011 ± 0.041* | 0.012 ± 0.042 | 0.002 ± 0.031** | 0.003 ± 0.052* | 0.006 ± 0.051** | 0.007 ± 0.047** | 0.001 ± |
| | | (8 mg/kg) | | | | | | | 0.041** |
| | | mPEG- | 0.007 ± 0.013* | 0.006 ± 0.006* | 0.026 ± 0.023 | 0.004 ± 0.006 | 0.011 ± 0.004* | 0.001 ± 0.021** | 0.023 ± |
| | | SC₂₀ₖ-HM-3 | | | | | | | 0.007 |
| | | (20 mg/kg) | | | | | | | |

vs. model groups,
*p < 0.05,
**p < 0.01.

TABLE 29
The impact of mPEG-SC₂₀ₖ-HM-3 on CIA chronic inflammation of right ankle swelling changes in TNF-luc mouse (mean ± SD, N = 2, cm)

| Groups | N | 0 d | 12 d | 15 d | 18 d | 21 d | 24 d | 27 d | 30 d |
|---|---|---|---|---|---|---|---|---|---|
| Normal | 2 | 0.000 ± 0.000 | 0.000 ± 0.008 | 0.005 ± 0.001 | 0.013 ± 0.009 | 0.005 ± 0.004 | 0.008 ± 0.006* | 0.004 ± 0.006** | 0.015 ± 0.004** |
| Model | 2 | 0.000 ± 0.000 | 0.004 ± 0.006 | 0.006 ± 0.004 | 0.012 ± 0.019 | 0.012 ± 0.002 | 0.022 ± 0.004 | 0.042 ± 0.000 | 0.046 ± 0.018 |
| Adalimumab | 2 | 0.000 ± 0.000 | 0.010 ± 0.014 | 0.000 ± 0.028 | 0.012 ± 0.011 | 0.007 ± 0.010 | 0.009 ± 0.007* | 0.012 ± 0.017* | 0.008 ± 0.008** |
| mPEG- | 2 | 0.000 ± 0.000 | 0.005 ± 0.021 | 0.003 ± 0.004 | 0.011 ± 0.016 | 0.010 ± 0.011 | 0.007 ± 0.005 | 0.004 ± 0.014** | 0.015 ± 0.001** |
| SC₂₀ₖ-HM-3 | | | | | | | | | |
| | | Groups | 33 d | 36 d | 39 d | 42 d | 45 d | 48 d | 51 d |
| | | Normal | 0.010 ± 0.008* | 0.014 ± 0.014** | 0.011 ± 0.001* | 0.006 ± 0.008** | 0.002 ± 0.011** | 0.008 ± 0.011** | 0.002 ± 0.003* |
| | | Model | 0.036 ± 0.004 | 0.045 ± 0.004 | 0.038 ± 0.010 | 0.040 ± 0.000 | 0.044 ± 0.010 | 0.044 ± 0.000 | 0.032 ± 0.000 |
| | | Adalimumab | 0.011 ± 0.016* | 0.017 ± 0.007* | 0.007 ± 0.010** | 0.012 ± 0.017** | 0.005 ± 0.021** | 0.006 ± 0.006** | 0.004 ± 0.006** |
| | | mPEG- | 0.018 ± 0.020* | 0.006 ± 0.008** | 0.001 ± 0.007** | 0.017 ± 0.004** | 0.000 ± 0.006** | 0.001 ± 0.007** | 0.005 ± 0.007** |
| | | SC₂₀ₖ-HM-3 | | | | | | | |

vs. model group,
*p < 0.05,
**p < 0.01.

TABLE 30
The impact changes of mPEG-SC₂₀ₖ-HM-3 on CIA chronic model of arthritis index score (mean ± SD, n = 2)

| Groups | N | 30 d | 33 d | 36 d | 39 d | 42 d | 45 d | 48 d | 51 d |
|---|---|---|---|---|---|---|---|---|---|
| Normal | 2 | 0.00 ± 0.00** | 0.00 ± 0.00** | 0.00 ± 0.00** | 0.00 ± 0.00** | 0.00 ± 0.0**0 | 0.00 ± 0.00** | 0.00 ± 0.00** | 0.00 ± 0.00** |
| Model | 2 | 5.00 ± 2.83 | 7.00 ± 0.00 | 9.00 ± 1.41 | 8.00 ± 0.00 | 8.50 ± 0.71 | 8.00 ± 1.41 | 7.00 ± 1.41 | 6.50 ± 0.71 |
| Adalimumab | 2 | 5.00 ± 2.83 | 8.00 ± 0.00 | 8.50 ± 0.71 | 6.50 ± 0.71* | 5.50 ± 0.71** | 5.00 ± 0.00** | 5.00 ± 1.41** | 4.50 ± 0.71** |
| mPEG- | 2 | 5.50 ± 0.71 | 7.00 ± 1.41 | 6.50 ± 0.71** | 6.00 ± 0.00** | 4.50 ± 0.71** | 4.50 ± 0.71** | 4.50 ± 0.71** | 4.50 ± 0.71** |
| SC₂₀ₖ-HM-3 | | | | | | | | | |

vs. model group,
*p < 0.05,
**p < 0.01.

TABLE 31
The impact changes of mPEG-SC₂₀ₖ-HM-3 on CIA chronic model of clinical score (mean ± SD, n = 2)

| Groups | N | 30 d | 33 d | 36 d | 39 d | 42 d | 45 d | 48 d | 51 d |
|---|---|---|---|---|---|---|---|---|---|
| Normal | 2 | 0.00 ± 0.00** | 0.00 ± 0.00** | 0.00 ± 0.00** | 0.00 ± 0.00** | 0.00 ± 0.00** | 0.00 ± 0.00** | 0.00 ± 0.00** | 0.00 ± 0.00** |
| Model | 2 | 5.00 ± 1.41 | 5.50 ± 0.71 | 5.50 ± 0.71 | 5.00 ± 0.00 | 6.00 ± 1.41 | 6.00 ± 1.41 | 6.00 ± 0.00 | 6.00 ± 0.00 |
| Adalimumab | 2 | 5.00 ± 1.41 | 5.00 ± 0.00 | 5.00 ± 0.00 | 5.00 ± 0.00 | 5.00 ± 0.00 | 4.00 ± 0.00** | 5.00 ± 0.00 | 4.50 ± 0.71* |
| mPEG- | 2 | 4.50 ± 0.71* | 4.00 ± 0.00** | 4.00 ± 0.00** | 4.00 ± 0.00** | 4.00 ± 0.00** | 4.00 ± 0.00** | 5.00 ± 0.00 | 4.00 ± 0.00** |
| SC₂₀ₖ-HM-3 | | | | | | | | | |

vs. model group,
*p < 0.05,
**p < 0.01.

TABLE 32
The impact changes of mPEG-SC₂₀ₖ-HM-3 on CIA chronic model of clinical score (mean ± SD, n = 2, kg)

| Groups | N | 0 d | 12 d | 15 d | 18 d | 21 d | 24 d | 27 d | 30 d |
|---|---|---|---|---|---|---|---|---|---|
| Normal | 2 | 0.000 ± 0.000 | 2.700 ± 0.400** | 3.500 ± 0.780** | 4.200 ± 0.200** | 4.460 ± 0.300** | 3.900 ± 0.770** | 4.200 ± | 4.400 ± |
| | | | | | | | | 1.180** | 1.870** |
| Model | 2 | 0.000 ± 0.000 | 0.600 ± 0.283 | 1.090 ± 0.438 | 0.730 ± 0.750 | 0.780 ± 0.679 | 1.435 ± 0.940 | 1.195 ± | 2.090 ± |
| | | | | | | | | 0.021 | 0.311 |
| Adalimumab | 2 | 0.000 ± 0.000 | 1.050 ± 0.778 | 0.940 ± 1.047 | 0.455 ± 0.247 | 0.230 ± 0.523 | 0.550 ± 0.212 | 1.295 ± | 0.975 ± |
| | | | | | | | | 0.064 | 0.049 |
| mPEG- | 2 | 0.000 ± 0.000 | 0.830 ± 0.523 | 1.000 ± 0.141 | 0.900 ± 0.141 | 1.435 ± 0.474 | 1.850 ± 0.778 | 1.455 ± | 1.230 ± |
| SC₂₀ₖ-HM-3 | | | | | | | | 1.025 | 1.428 |
| | | Groups | 33 d | 36 d | 39 d | 42 d | 45 d | 48 d | 51 d |
| | | Normal | 4.010 ± 1.170** | 5.060 ± 1.640** | 5.170 ± 1.150** | 4.700 ± 0.900** | 4.910 ± 1.030** | 6.370 ± | 7.080 ± |
| | | | | | | | | 1.400** | 1.640** |
| | | Model | 1.575 ± 0.573 | 1.890 ± 0.354 | 1.955 ± 1.138 | 2.075 ± 1.662 | 2.510 ± 2.093 | 2.630 ± | 2.610 ± |
| | | | | | | | | 1.739 | 1.372 |
| | | Adalimumab | 1.295 ± 0.177 | 2.200 ± 0.707 | 2.187 ± 0.443 | 2.655 ± 1.082* | 3.085 ± 1.478* | 3.415 ± | 3.835 ± |
| | | | | | | | | 1.846** | 1.435** |
| | | mPEG- | 1.335 ± 0.431 | 1.389 ± 1.331 | 2.950 ± 0.028** | 3.100 ± 0.849** | 3.655 ± 1.138** | 3.685 ± | 4.735 ± |
| | | SC₂₀ₖ-HM-3 | | | | | | 1.308** | 1.322** |

vs. model group,
*p < 0.05,
**p < 0.01.

What is claimed is:

1. A polypeptide for use in a method for treatment or prevention of rheumatoid arthritis by a combination of downregulating inflammatory response and anti-angiogenesis, wherein the amino acid sequence of said polypeptide is:
mPEG-SC₂₀ₖ-Ile-Val-Arg-Arg-Ala-Asp-Arg-Ala-Ala-Val-Pro-Gly-Gly-Gly-Gly-Arg-Gly-Asp (polypeptide III (SEQ ID NO: 3)).

2. The polypeptide for use according to claim 1, wherein the polypeptide III (SEQ ID NO: 3) is prepared by means of synthesis or recombination of expression vectors.

3. The polypeptide for use according to claim 1, wherein the polypeptide III (SEQ ID NO: 3) is configured to covalently couple to an adjuvant; wherein the adjuvant is selected from bovine serum albumin, human serum albumin or polyethylene glycol (PEG).

4. The polypeptide for use according to claim 1, wherein said use involves the preparation of drugs for treatment or prevention of rheumatoid arthritis, wherein said drugs for treatment or prevention of rheumatoid arthritis contain an effective amount of salts acceptable to polypeptide III (SEQ ID NO: 3), or if necessary, pharmaceutically acceptable vectors or excipients.

5. The polypeptide for use according to claim 1, wherein said use involves the preparation of drugs for treatment or prevention of rheumatoid arthritis, wherein said drugs for treatment or prevention of rheumatoid arthritis can be administered through a variety of routes, including hypodermic injection, intramuscular injection, intravenous injection or drip, oral administration, including in the form of pills, capsules, and nasal spray.
